(19) United States
(12) Patent Application Publication
(10) Pub. No.: US 2008/0103191 A1
(43) Pub. Date: May 1, 2008

(54) ENTACAPONE-DERIVATIVES

(76) Inventors: Klaus Hansen, Grevenbroich-Munchrath (DE); Robert Kraemer, Koln (DE); Thomas Lauterbach, Dusseldorf (DE); Dirk Schmidt, Monheim (DE); Fritz Paar, Neuss (DE); Petra Pfeiffer, Solingen (DE); Detlef Geffken, Luneburg (DE); Wolfgang Thimann, Hamburg (DE); Katharina Wehner, Hamburg (DE)

Correspondence Address: HARNESS, DICKEY & PIERCE, P.L.C., P.O. BOX 828, BLOOMFIELD HILLS, MI, 48303, US

(21) Appl. No.: 11/763,028

(22) Filed: Jun. 14, 2007

(30) Foreign Application Priority Data
Jun. 16, 2006 (EP) EP06012394.0

(51) Int. Cl. A61K 31/265; A61K 31/341; A61K 31/357; A61K 31/40; A61P 25/00; C07C 69/96; C07D 207/09; C07D 307/33; C07D 317/30

(52) U.S. Cl. 514/423; 514/467; 514/473; 514/512; 548/531; 549/318; 549/454; 558/267; 558/271

(57) ABSTRACT

Pharmaceutical composition comprising one or more entacapone derivatives and one or more pharmaceutically acceptable carriers, a process for producing the pharmaceutical composition, specific entacapone derivatives, a process for the preparation of entacapone derivatives, and the use of the entacapone derivatives for the preparation of a medicament.

### CROSS-REFERENCE TO RELATED APPLICATION

This application claims the benefit of European Application No. EP 06012394.0, filed Jun. 16, 2006. The disclosure of the above application is incorporated herein by reference.

### FIELD OF INVENTION

The present invention relates to a pharmaceutical composition comprising one or more entacapone derivatives and one or more pharmaceutically acceptable carriers, a process for producing the pharmaceutical composition, specific entacapone derivatives, a process for the preparation of entacapone derivatives, a process for the preparation of entacapone, and the use of the entacapone derivatives for the preparation of a medicament, preferably for the treatment and/or prophylaxis of diseases associated with a disordered dopamine metabolism or an altered enzyme activity of COMT.

### TECHNICAL BACKGROUND

Parkinson's disease is believed to be primarily caused by the degeneration of dopaminergic neurons in the substantia nigra. This, in effect, results in loss of tonic dopamine secretion and dopamine-related modulation of neuronal activity in the caudate nucleus, and in a deficiency of dopamine in other brain regions.

Treatment of Parkinson's disease has been attempted with, inter alia, L-dopa (levodopa), which is a precursor of dopamine, of which there is a shortage in the brains of patients suffering from Parkinson's disease. L-dopa is mainly metabolized by COMT (catechol-O-methyltransferase) and aromatic amino acid decarboxylase (AADC). L-dopa is therefore used in the treatment of Parkinson's disease usually in combination with an AADC inhibitor as well as a COMT-inhibitor. As COMT-inhibitor entacapone ((E)-N,N-diethyl-2-cyano-3-(3,4-dihydroxy-5-nitrophenyl)acrylamide) is used. Entacapone enhances the bioavailability of L-dopa and extends its effective period. Because of this effect it is possible to reduce the dose of L-dopa by 10 to 30%. There is evidence that the longer duration of the effects of L-dopa leads to a more constant stimulation of dopamine receptors which may reduce the extent of motor complications associated with short acting L-dopa.

Entacapone and derivatives thereof as well as their suitability as COMT-inhibitors are described in U.S. Pat. No. 4,963,590, U.S. Pat. No. 5,112,861, U.S. Pat. No. 5,283,352, and U.S. Pat. No. 5,446,194. Polymorphic forms of entacapone are described in U.S. Pat. No. 5,135,950, WO 2005/063696, and WO 2005/070881.

Further, EP 1 189 608 B1 describes pharmaceutical compositions comprising entacapone, L-dopa, and the decarboxylase inhibitor carbidopa.

None of the documents mentioned before concerns the bioavailability of entacapone, which is relatively low.

J. Leppänen et al., Pharmacy and Pharmacology 2001, 53, 1489-1498, discuss investigations concerning the oral bioavailability of entacapone derivatives. Therefore, acyl and acyloxyacyl esters, an acyloxyalkyl ether and an alkyloxycarbonyl ester of entacapone have been synthesized and evaluated as potential prodrugs of entacapone. Only two acyl esters, the monopivaloyl ester and the dipivaloyl ester of entacapone, which appeared as most promising entacapone derivatives, have been tested in vivo. However, it has been found that the lipophilic entacapone prodrugs tested were not able to improve the oral bioavailability in rats.

Thus, there is still a need for entacapone derivatives that can be used to, for example, improve the oral bioavailability of entacapone. There is also a need for compounds that can be used as, for example, COMT inhibitors. There is further a need for compounds that can be used to, for example, prevent and/or treat diseases associated with disordered dopamine metabolism and/or altered catechol-O-methyltransferase activity. This invention generally provides such compounds.

### BRIEF DESCRIPTION OF THE DRAWINGS

The drawings described herein are for illustration purposes only and are not intended to limit the scope of the present disclosure in any way.

FIG. 1 shows pharmacokinetics of one of the compounds of the invention.

### DETAILED DESCRIPTION

The present invention provides a pharmaceutical composition comprising one or more compounds of formula I, or a salt thereof. In addition, the invention relates to compounds, to mixtures or combinations of compounds and salts, stereoisomers, solvates, hydrates, enantiomers, diasteromers, and isotopically labelled derivatives thereof.

In one embodiment, the compounds correspond in structure to formula I:

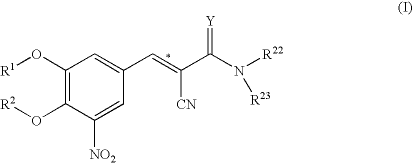

wherein
Y is sulfur or oxygen,
R¹ is a group of the following formula II, or when Y is S, R¹ can be in addition H

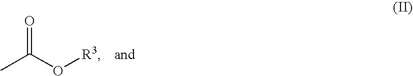

R² is H or a group of formula II which may be the same as or different from R¹.

As used herein, R represents organic groups that complete the organic carbonate ester groups at position 3 (“meta” to the nitro group) and optionally position 4 (“ortho” to the nitro group) of the phenyl ring in the 2-cyanopropenamide structures shown, and which can be incorporated into the structure by any of the methods known in the art, including those described herein. As such, they are selected from a wide variety of moieties containing carbon, hydrogen, and optionally oxygen, nitrogen, sulfur, and other heteroatoms. If R³ is present in both R¹ and R², the R³ groups can be the same or different.

In one aspect, the size (or, equivalently, the molecular weight) of R³ is not particularly limited. However, for practical purposes such as solubility, ease of synthesis, molar bioavailability and similar considerations, the group or groups R³ generally contain 100 or fewer total non-hydrogen atoms, preferably 50 total non-hydrogen atoms or less, and in many cases 20 non-hydrogen atoms or less. Thus in various embodiments, the groups R³ are independently selected from organic (carbon containing) groups having from 1 to about 50, preferably 1 to about 20 non-hydrogen atoms, it being understood that at least one of the non-hydrogen atoms of R³ is a carbon. In many embodiments, over half of the non-hydrogen atoms are carbon. In selecting suitable groups R³, naturally certain combinations of atoms, functional groups, and structural features are less preferred if they tend to be poisonous (e.g. heavy metals), highly reactive (e.g. isocyanates, isothiocyanates, acid chlorides) or structurally unstable (e.g. certain acetals). In another aspect, the group R³ is described by the number of carbon atoms rather than the number of non-hydrogens. In various embodiments, R³ has at least one and a maximum of 50, 40, 35, 30, 25, 20, or 15 carbon atoms. To illustrate, the following further definitions are given for Formula I:
each R³ is independently alkyl, (CR⁴R⁵)ₓ—R⁶, alkylene-alkoxy, alkenyl, alkynyl, alkylene-cycloalkyl, alkylene-heterocycloalkyl, alkylene-cycloalkenyl, alkylene-heterocycloalkenyl, alkylene-aryl, alkylene-heteroaryl, alkoxy, cycloalkyl, heterocycloalkyl, cycloalkenyl, heterocycloalkenyl, aryl, heteroaryl, alkenylene-cycloalkyl, alkenylene-heterocycloalkyl, alkenylene-cycloalkenyl, alkenylene-heterocycloalkenyl, alkenylene-aryl, or alkenylene-heteroaryl,
R⁴ and R⁵ are independently of one another selected from the group consisting of H, alkyl, alkylene-hydroxy, alkylene-alkoxy, OH, alkylene-N(R⁷)CO-alkyl, alkylene-CON(R⁸)(R⁹), alkylene-COO-alkyl, alkylene-N(R¹⁰)(R¹¹), SO₃R¹⁷, alkylene-aryl, alkylene-heteroaryl, alkoxy, N(R⁷)CO-alkyl, CON(R⁸)(R⁹), COO-alkyl, N(R¹⁰)(R¹¹), aryl, and heteroaryl,
   or

### R⁴ and R⁵ of the same group (CR⁴R⁵) or R⁴ and R⁵ of different groups (CR⁴R⁵) may form together a carbocyclic or heterocyclic ring,

additionally, one or more non adjacent groups (CR⁴R⁵) may be replaced by O, CO, OCO, COO, CON(R¹⁹), N(R²⁰)CO, or NR²¹,
R⁶ is independently H, alkyl, alkenyl, alkynyl, OH, O-alkyl, O-alkylene-aryl, O-aryl, CO—O-alkyl, CO—N(R¹²)(R¹³), N(R¹⁴)CO-alkyl, N(R¹⁵)(R¹⁶), SO₃R¹⁸, alkylene-heteroaryl, heteroaryl, alkylene-aryl, or aryl,
R⁷, R¹⁴, R¹⁷, R¹⁸, R¹⁹, R²⁰, R²¹
   are independently of one another H, or alkyl,
R⁸, R⁹, R¹⁰, R¹¹, R¹², R¹³, R¹⁵, R¹⁶
   are independently of one another H, or alkyl,
R²² and R²³ are independently selected from the group consisting of H and alkyl, and
x is 1 to 14,
   wherein the alkyl, alkenyl, alkynyl, cycloalkyl, heterocycloalkyl, cycloalkenyl, heterocycloalkenyl, alkoxy, aryl, heteroaryl, alkenylene and alkylene groups may be unsubstituted or further substituted;
   and one or more pharmaceutically acceptable carriers.

As drawn above and at other places in the specification, the compounds are shown formally in an E configuration about the aliphatic double bond of the 2-cyanopropenamide structures shown. In various embodiments, the compounds have a Z configuration, or they are a mixture of compounds containing both the Z and E isomers. Such compounds can be represented by the structure

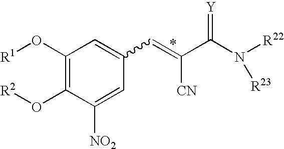

wherein the substituents are defined herein and the wavy bond indicates either a Z, E, or mixture of double bond configurations. In various embodiments, the compounds of the invention are provided in combinations of Z and E isomers where the E isomer is present in a higher amount. Preferred compounds of formula I are the E-isomers. In some embodiments, the compounds are substantially pure in the stereoisomer sense, meaning that 90% or more of the molecules are in one or other of the configurations. The compounds include pure E isomers, pure Z isomers, and combinations of the two in any proportion. The isomeric forms may be obtained by known methods.

In some embodiments, a compound of formula I is in the form of a mixture of E and Z isomers with regard to the double bond labelled with an asterisk in formula I. Preferably, the mixture is enriched in the E isomer.

In some embodiments, a compound of formula I is in the form of a Z isomer with regard to the double bond labelled with an asterisk. Preferably, a compound of formula I is in the form of an E isomer with regard to the double bond labelled with an asterisk. As drawn, formula I shows an E stereoisomer.

Unless a name of compound specifically indicates that the compound is in the form of a specific isomer, the term “compound”, a name of a compound, or a structural formula of a compound encompasses all possible isomers of the compound as well as all possible mixtures of isomers of the compound. Illustratively, the name “N,N-diethyl-2-cyano-3-(3,4-dihydroxy-5-nitrophenyl)acrylamide” can encompass an E isomer, a Z isomer, or a mixture of the E and Z stereoisomers. Illustrating further, structural formula I can encompass a compound which is in the form of an E isomer with regard to the double bond labelled with an asterisk, a compound which is in the form of a Z isomer with regard to the double bond labelled with an asterisk, and a compound which is in the form of a mixture of the E and Z isomers with regard to the double bond labelled with an asterisk.

Further, the compounds of formula I may be in the form of their racemates, enantiomer-enriched mixtures and pure enantiomers and to their diastereomers and mixtures thereof in the case that the compound of formula I comprises one or more centers of asymmetry. The isomeric forms may be obtained by known methods, even if not expressly described.

In a preferred embodiment the present invention relates to a pharmaceutical composition comprising one or more compounds or formula I or a salt thereof and one or more pharmaceutically acceptable carriers,

wherein
Y is sulphur or oxygen
R¹ is a group of the following formula II

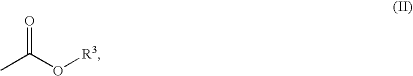

R² is H or a group of formula II which may be the same as or different from R¹,
each R³ is independently (C₁-C₂₀)-alkyl, (CR⁴R⁵)ₓ—R⁶, (C₁-C₂₀)-alkylene-(C₁-C₂₀)-alkoxy, (C₂-C₂₀)-alkenyl, (C₂-C₂₀)-alkynyl, (C₀C₂₀)-alkylene-(C₃-C₁₈)-cycloalkyl, (C₀-C₂₀)-alkylene-(3-18-membered)-heterocycloalkyl, (C₁-C₂₀)-alkylene-(C₃-C₁₈)-cycloalkenyl, (C₀-C₂₀)-alkylene-(3-18-membered)-hetero-cycloalkenyl, (C₀-C₂₀)-alkylene-(C₆-C₁₈)-aryl, (C₀-C₂₀)-alkylene-(5-18-membered)-heteroaryl, (C₂-C₂₀)-alkenylene-(C₃-C₁₈)-cycloalkyl, (C₂-C₂₀)-alkenylene-(3-18-membered)-heterocycloalkyl, (C₂-C₂₀)-alkenylene-(C₃-C₁₈)-cycloalkenyl, (C₂-C₂₀)-alkenylene-(3-18-membered)-heterocycloalkenyl, (C₂-C₂₀)-alkenylene-(C₆-C₁₈)-aryl, or (C₂-C₂₀)-alkenylene-(5-18-membered)-heteroaryl,
   wherein the total number of carbon atoms of R³ is at most 30, preferably at most 25, more preferably at most 15
each R⁴ and R⁵
   are independently of one another selected from the group consisting of H, (C₁-C₂₀)-alkyl, (C₁-C₂₀)-alkylene-hydroxy, (C₀-C₂₀)-alkylene-(C₁-C₂₀)-alkoxy, OH, (C₀-C₂₀)-alkylene-N(R⁷)CO—(C₁-C₂₀)-alkyl, (C₀-C₂₀)-alkylene-CON(R⁸)(R⁹), (C₀-C₂₀)-alkylene-COO—(C₁-C₂₀)-alkyl, (C₀-C₂₀)-alkylene-N(R¹⁰)(R¹¹), SO₃R¹⁷, (C₀-C₂₀)-alkylene-(C₆-C₁₈)-aryl, and (C₀-C₂₀)-alkylene-(5-18-membered)-heteroaryl,
      or

### R⁴ and R⁵ of the same group (CR⁴R⁵) or R⁴ and R⁵ of different groups (CR⁴R⁵) may form together a carbocyclic or heterocyclic ring having from 3 to 6 atoms,

additionally, one or more non adjacent groups (CR⁴R⁵) may be replaced by O, CO, OCO, COO, CON(R¹⁹), N(R²⁰)CO, or NR²¹,
R⁶ is independently H, (C₁-C₂₀)-alkyl, (C₂-C₂₀)-alkenyl, (C₂-C₂₀)-alkynyl, OH, O—(C₁-C₈)-alkyl, O—(C₀-C₈)-alkylene-(C₆-C₁₄)-aryl, CO—O—(C₁-C₈)-alkyl, CO—N(R¹²)(R¹³), N(R¹⁴)CO—(C₁-C₈)-alkyl, N(R¹⁵)(R¹⁶), SO₃R¹⁸, (C₀-C₂₀)-alkylene-(5-18-membered)-heteroaryl, or (C₀-C₂₀)-alkylene-(C₆-C₁₈)-aryl,
R⁷, R¹⁴, R¹⁷, R¹⁸, R¹⁹, R²⁰, R²¹
   are independently of one another H, or (C₁-C₂₀)-alkyl,
R⁸, R⁹, R¹⁰, R¹¹, R¹², R¹³, R¹⁵, R¹⁶
   are independently of one another H, or (C₁-C₂₀)-alkyl,
R²² and R²³ are independently selected from the group consisting of H and (C₁-C₁₅)-alkyl, and
x is 1 to 14,
   wherein the alkyl, alkenyl, alkynyl, cycloalkyl, heterocycloalkyl, cycloalkenyl, heterocycloalkenyl, alkoxy, aryl, heteroaryl, alkenylene and alkylene groups may be unsubstituted or further substituted;
   and one or more pharmaceutically acceptable carriers.

In some embodiments the present invention relates to a pharmaceutical composition comprising one or more compounds of formula I or a salt thereof and one or more pharmaceutically acceptable carriers,

wherein
Y is oxygen, and
R²² and R²³ are ethyl.

The compounds according to this embodiment can be represented by the structure:

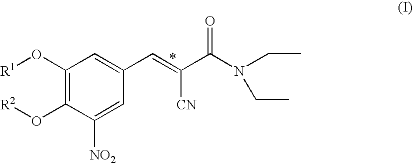

Suitable salts of the compounds of formula I usually have a pharmaceutically acceptable anion or cation. Suitable pharmaceutically acceptable acid addition salts of the compounds of formula I are salts of inorganic acids such as hydrochloric acid, hydrobromic, phosphoric, metaphosphoric, nitric and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic and tartaric acid. Suitable pharmaceutically acceptable basic salts are ammonium salts, alkali metal salts (such as sodium and potassium salts), alkaline earth metal salts (such as magnesium and calcium salts) and salts of trometamol (2-amino-2-hydroxymethyl-1,3-propanediol), diethanolamine, lysine or ethylenediamine.

Salts with a pharmaceutically unacceptable anion, such as, for example, trifluoro acetate, likewise belong within the framework of the invention as useful intermediates for the preparation or purification of pharmaceutically acceptable salts and/or for use in nontherapeutic, for example in vitro, applications.

The term “alkyl” as employed in the present invention by itself or as part of another group includes both straight and branched saturated hydrocarbyl chain radicals of up to 20 carbon atoms, preferably 1 to 15 carbon atoms, more preferably 1 to 10 carbon atoms, most preferably 1 to 8 carbon atoms, still more most preferably 1 to 4 carbon atoms. Specific examples for the alkyl residues are methyl, ethyl, n-propyl, isopropyl, n-butyl, isobutyl, sec-butyl, t-butyl, pentyl, 1-ethyl-propyl, iso-amyl, hexyl, octyl, decyl, and dodecyl including the various branched and straight chain isomers thereof. The alkyl group may be substituted or unsubstituted.

When a carbon atom number range is given with a minimum of zero (e.g. (C₀-C₂₀)-alkyl), it is to be understood that the recitation encompasses embodiments with zero carbons, which means the substituent is not present. To illustrate, (C₀-C₂₀)-alkylene-(C₃-C₁₈)-cycloalkyl includes (C₃-C₁₈)-cycloalkyl and (C₁-C₂₀)-alkylene-(C₃-C₁₈)-cycloalkyl, and is to be interpreted as appropriate as a recitation of both.

The terms “alkenyl” and “alkynyl” include straight and branched chain radicals of up to 20 carbon atoms, preferably 2 to 15 carbon atoms, more preferably 2 to 8 carbon atoms, most preferably 2 to 6 carbon atoms, wherein the hydrocarbon chain comprises at least one carbon to carbon double bond (in the case of “alkenyl”) respectively at least one carbon to carbon triple bond (in the case of “alkynyl”). The alkenyl group and the alkynyl group may be substituted or unsubstituted. Examples of “alkenyl” substituents include ethenyl (vinyl), 2-propenyl, 3-propenyl, 1,4-pentadienyl, 1,4-butadienyl, 1-butenyl, 2-butenyl, 3-butenyl, pentenyl, hexenyl, and octenyl. Examples of “alkynyl” substituents include ethynyl, 2-propynyl, 3-propynyl, 1-butynyl, 2-butynyl, 3-butynyl, pentynyl, hexynyl, and octynyl.

The term “alkylene” refers to a straight or branched saturated chain containing from 1 to 20 carbon atoms, preferably 1 to 15 carbon atoms, more preferably 1 to 8, most preferably 1 to 4, still more most preferably 1 or 2 carbon atoms. The alkylene group may be substituted or unsubstituted.

The term “alkenylene” refers to a straight or branched chain containing from 2 to 20 carbon atoms, preferably 2 to 15 carbon atoms, more preferably 2 to 8, most preferably 2 to 4, still more most preferably 2 or 3 carbon atoms, wherein the alkenylene-chain comprises at least one carbon to carbon double bond. The alkenylene group may be substituted or unsubstituted.

The term “cycloalkyl” refers to a cyclic alkyl group comprising 3 to 18 ring carbon atoms, preferably 3 to 14 ring carbon atoms, more preferably 5 to 10 ring carbon atoms. A cycloalkyl may be a single carbon ring, which typically contains from 3 to 6 carbon ring atoms. Examples of single-ring cycloalkyls include cyclopropyl, cyclobutyl, cyclopentyl, and cyclohexyl. A cycloalkyl alternatively may be a fused, bridged or spirocyclic ring system of 2 or 3 rings such as, for example, norbonyl, decalinyl, bicycloheptanyl, adamantyl, and norpinanyl. The cycloalkyl group may be substituted or unsubstituted.

The term “heterocycloalkyl” includes cyclic alkylene groups, wherein one or more carbon atoms of the cycloalkyl ring as defined before concerning the term “cycloalkyl” are replaced by a heteroatom, for example O, S and/or a group comprising a heteroatom, for example CO, NR²¹. The heterocycloalkyl group comprises 3 to 18 ring atoms, preferably 3 to 14 ring atoms, more preferably 5 to 10 ring atoms. A heterocycloalkyl alternatively may be a fused, bridged or spirocyclic ring system of 2 or 3 rings. In a preferred embodiment the “heterocycloalkyl” group is bound via a carbon ring atom. Specific examples are piperidine, pyrrolidine, tetrahydrofurane, thiolane, butylrolactone, oxane, oxanone, dioxolanone and dioxolane groups. The heterocycloalkyl group may be substituted or unsubstituted.

The term “cycloalkenyl” refers to a cyclic alkenyl group which is defined as the cycloalkyl group mentioned before, additionally comprising at least one C═C-double bond. The cycloalkenyl group may be substituted or unsubstituted.

The term “heterocycloalkenyl” refers to a cyclic alkenyl group comprising at least one heteroatom which is defined as the “heterocycloalkyl” group mentioned before, additionally comprising at least one double bond. Suitable heterocycloalkenyl groups are for example dihydrofurane, or dihydrofuranone. The heterocycloalkenyl group may be substituted or unsubstituted.

The term “alkoxy” as employed in the present invention by itself or as part of another group includes an alkyl residue as defined above linked to an oxygen atom. Preferred alkoxy groups comprise 1 to 20 carbon atoms, preferably 1 to 15 carbon atoms, more preferably 1 to 10 carbon atoms, most preferably 1 to 8 carbon atoms, most preferably 1 to 4 carbon atoms.

The term “aryl” as used herein by itself or as part of another group refers to an aryl group preferably being a monocyclic or bicyclic group containing from 6 to 18 ring carbon atoms, preferably from 6 to 14 ring carbon atoms, more preferably from 6 to 10 ring carbon atoms. It is possible, that the aryl groups in the meaning of the present invention comprise one aromatic and one non-aromatic ring. Specific examples for aryl groups are phenyl, naphthyl and indenyl. The aryl group may be substituted or unsubstituted.

The term “heteroaryl” as employed in the present invention refers to monocyclic or bicyclic aromatic groups containing 1 to 3, preferably 1 or 2, more preferably 1 heteroatom, especially N and/or O and/or S. The heteroaryl group contains 5 to 18 ring atoms, preferably from 5 to 14 ring atoms, more preferably from 5 to 10 ring atom Preferably, the “heteroaryl” group is bound via a carbon ring atom. It is possible, that the heteroaryl groups in the meaning of the present invention comprise one aromatic and one non-aromatic ring. Specific examples of heteroaryl substituents include 6-membered ring substituents such as pyridinyl, pyrazinyl, pyrimidinyl, pyridazinyl, and 1,3,5-, 1,2,4-, and 1,2,3-triazinyl; 5-membered ring substituents such as thienyl, imidazolyl, furanyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzodioxolyl, benzisoxazolyl, benzoxazolyl, purinyl, and anthranilyl; and 6/6-membered fused rings such as quinolinyl, isoquinolinyl, cinnolinyl, and quinazolinyl. The heteroaryl group may be substituted or unsubstituted.

A carbocyclic ring as described herein is a cycloalkyl, cycloalkenyl or aryl, preferably a cycloalkyl.

A heterocyclic ring as described herein is a heterocycloalkyl, heterocycloalkenyl, or a heteroaryl. Preferably a heterocyclic ring according to a compound of formula I of the invention is a heterocycloalkyl.

The alkyl, alkenyl, alkynyl, carbocyclic ring, cycloalkyl, cycloalkenyl, heterocyclic ring, heterocycloalkyl, heterocycloalkenyl, alkoxy, aryl, and heteroaryl groups as well as the alkylene and alkenylene groups mentioned above may be further substituted or unsubstituted. In a preferred embodiment of the invention alkyl, aryl, cycloalkyl, heterocycloalkyl and heterocycloalkenyl may be further substituted or unsubstituted. In a preferred embodiment of the invention, the above defined groups that are optionally substituted with one or more independently selected substituents as defined below, are residues—or a part of a residue—at R³ to R²¹ of a compound according to formula I. In a more preferred embodiment of the invention, the above defined groups that are optionally substituted with one or more independently selected substituents as defined below, are residues—or a part of a residue—at R³, R⁴, R⁵ and/or R⁶ of a compound according to formula I. In an even more preferred embodiment of the invention, the groups that are optionally substituted with one or more independently selected substituents as defined below, are residues—or a part of a residue—at R³ of a compound according to formula I.

Suitable substituents are for example halogen substituents such as fluorine, chlorine, bromine, iodine, or trifluoromethyl groups, ═CH₂, (C₁-C₂₀)-alkyl, (C₀-C₂₀)-alkylene-(C₁-C₂₀)-alkoxy, (C₀-C₂₀)-alkylene-(C₆-C₁₈)-aryl, (C₀-C₂₀)-alkylene-(5-18-membered)-heteroaryl, halogenated (C₆-C₁₈)-aryl, halogenated (5-18-membered)-heteroaryl, (C₀-C₂₀)-alkylene-(C₅-C₁₈)-cycloalkyl, (C₀-C₂₀)-alkylene-(5-18-membered)-heterocycloalkyl, OH, SO₃R¹⁷, (C₀-C₂₀)-alkylene-N(R¹⁵)(R¹⁶), (C₀-C₂₀)-alkylene-CO—O—(C₁-C₁₉)-alkyl, (C₀-C₂₀)-alkylene-CO—OH, (C₀-C₂₀)-alkylene-CO—N(R¹²)(R¹³), (C₀-C₂₀)-alkylene-N(R¹⁴)—CO—(C₁-C₈)-alkyl, nitro, oxo, or cyano substituents. Preferred substituents are halogen substituents such as fluorine, chlorine, bromine, iodine, ═CH₂, (C₁-C₁₅)-alkyl, (C₀-C₁₅)-alkylene-(C₁-C₁₅)-alkoxy, (C₀-C₁₅)-alkylene-(C₆-C₁₈)-aryl, (C₀-C₁₅)-alkylene-(C₅-C₁₈)-cycloalkyl, OH, (C₀-C₁₅)-alkylene-N(R¹⁵)(R¹⁶), (C₀-C₁₅)-alkylene-CO—O—(C₁-C₈)-alkyl, (C₀-C₁₅)-alkylene-CO—OH, (C₀-C₁₅)-alkylene-CO—N(R¹²)(R¹³), (C₀-C₁₅)-alkylene-N(R¹⁴)—CO—(C₁-C₈)-alkyl, oxo, or nitro substituents. In a further preferred embodiment the substituents are selected from the group consisting of halogen substituents such as fluorine, chlorine, bromine, iodine, or trifluoromethyl groups, ═CH₂, (C₁-C₁₅)-alkyl, (C₀-C₁₅)-alkylene-(C₀-C₁₅)-alkoxy, OH, SO₃R¹⁷, (C₀-C₁₅)-alkylene-N(R¹⁵)(R¹⁶), (C₀-C₁₅)-alkylene-CO—O—(C₁-C₈)-alkyl, (C₀-C₁₅)-alkylene-CO—OH cyano, or oxo substituents. The number of substituents may be from 1 to 6, preferably 1 or 4, more preferably 1, 2 or 3.

If radicals or substituents occur more than once in the compounds of the formula I, they may all have the stated meanings independently of one another and be identical or different.

In a preferred embodiment of the present invention each R³ of a compound according to formula I is independently (C₁-C₁₅)-alkyl, (CR⁴R⁵)ₓ—R⁶, (C₁-C₁₅)-alkylene-(C₁-C₁₅)-alkoxy, (C₂-C₂₀)-alkenyl, (C₂-C₁₅)-alkynyl, (C₀-C₁₅)-alkylene-(C₃-C₁₈)-cycloalkyl, (C₀-C₁₅)-alkylene-(3-18-membered)-heterocycloalkyl, (C₁-C₁₅)-alkylene-(C₃-C₁₈)-cycloalkenyl, (C₀-C₁₅)-alkylene-(3-18-membered)-heterocycloalkenyl, (C₀-C₁₅)-alkylene-(C₆-C₁₈)-aryl, or (C₀-C₁₅)-alkylene-(5-18-membered)-heteroaryl, (C₂-C₁₅)-alkenylene-(C₃-C₁₈)-cycloalkyl, (C₂-C₁₅)-alkenylene-(3-18-membered)-heterocycloalkyl, (C₂-C₁₅)-alkenylene-(C₃-C₁₈)-cycloalkenyl, (C₂-C₁₅)-alkenylene-(3-18-membered)-heterocycloalkenyl, (C₂-C₁₅)-alkenylene-(C₆-C₁₈)-aryl, or (C₂-C₁₅)-alkenylene-(5-18-membered)-heteroaryl, wherein the total number of carbon atoms of R³ is at most 25,

preferably, each R³ is independently (C₁-C₁₀)-alkyl, (CR⁴R⁵)ₓ—R⁶, (C₁-C₈)-alkylene-(C₁-C₄)-alkoxy, (C₂-C₂₀)-alkenyl, (C₂-C₈)-alkynyl, (C₀-C₈)-alkylene-(C₃-C₁₄)-cycloalkyl, (C₀-C₈)-alkylene-(3-14-membered)-heterocycloalkyl, (C₀-C₈)-alkylene-(C₆-C₁₄)-aryl, or (C₀-C₈)-alkylene-(5-14-membered)-heteroaryl, wherein the total number of carbon atoms of R³ is at most 15,
more preferably, each R³ is independently (C₁-C₁₀)-alkyl, (CR⁴R⁵)ₓ—R⁶, (C₂-C₆)-alkenyl, (C₂-C₆)-alkynyl, (C₀-C₂)-alkylene-(C₅-C₁₀)-cycloalkyl, (C₀-C₂)-alkylene-(5-10-membered)-heterocycloalkyl, (C₀-C₂)-alkylene-(C₆-C₁₀)-aryl, or (C₀-C₂)-alkylene-(5-10-membered)-heteroaryl, wherein the total carbon number of R³ is at most 15,
most preferably (C₁-C₈)-alkyl, (CR⁴R⁵)ₓ—R⁶, (C₂-C₄)-alkenyl, (C₂-C₄)-alkynyl, (C₀-C₂)-alkylene-(C₆-C₁₀)-cycloalkyl, (C₀-C₂)-alkylene-(5-8-membered)-heterocycloalkyl, (C₀-C₂)-alkylene-(C₆-C₁₀)-aryl, (C₀-C₂)-alkylene-(5-10-membered)-heteroaryl, wherein the total carbon number of R³ is at most 15.

In a further embodiment of the present invention each R³ of a compound according to formula I is independently (C₁-C₁₀)-alkyl, (CR⁴R⁵)ₓ—R⁶, (C₁-C₈)-alkylene-(C₁-C₄)-alkoxy, (C₃-C₂₀)-alkenyl, (C₃-C₈)-alkynyl, (C₀-C₈)-alkylene-(C₃-C₁₄)-cycloalkyl, (C₀-C₈)-alkylene-(3-14-membered)-heterocycloalkyl, (C₀-C₈)-alkylene-(3-14-membered)-heterocycloalkenyl, (C₁-C₈)-alkylene-(C₃-C₁₄)-cycloalkenyl, (C₀-C₈)-alkylene-(C₆-C₁₄)-aryl, or (C₀-C₈)-alkylene-(5-14-membered)-heteroaryl, wherein the total carbon number of R³ is at most 15,

more preferably each R³ is independently (C₁-C₁₀)-alkyl, (CR⁴R⁵)ₓ—R⁶, (C₃-C₂₀)-alkenyl, (C₃-C₈)-alkynyl, (C₀-C₈)-alkylene-(C₃-C₁₄)-cycloalkyl, (C₀-C₈)-alkylene-(3-14-membered)-heterocycloalkyl, (C₀-C₈)-alkylene-(C₆-C₁₄)-aryl, or (C₀-C₈)-alkylene-(5-14-membered)-heteroaryl, wherein the total carbon number of R³ is at most 15.

In a further embodiment of the present invention each R³ of a compound according to formula I is independently selected from the group consisting of (C₁-C₄)-alkyl, preferably methyl, ethyl, n-propyl, isopropyl, n-butyl, isobutyl, sec-butyl; (C₅-C₇)-alkyl; preferably C₅-alkyl, and even more preferably 1-ethyl-propyl and (C₈-C₂₀)-alkyl, preferably (C₈-C₁₂-alkyl), more preferably (C₈-C₁₀)-alkyl, most preferably 2-ethylhexyl, n-octyl,

more preferably each R³ of a compound according to formula I is selected from the group consisting of (C₅-C₇)-alkyl; preferably C₅-alkyl,
even more preferably each R³ of a compound according to formula I is C₅-alkyl, preferably 1-ethyl-propyl,
even more preferably, each R³ of a compound according to formula I is selected from the group consisting of ethyl, isopropyl, isobutyl, 1-ethyl-propyl or R³ is 2-ethylhexyl.

In one embodiment of the present invention, in the case that R³ of a compound according to formula I is (C₈-C₂₀)-alkyl, preferably (C₈-C₁₂-alkyl), more preferably (C₈-C₁₀)-alkyl, most preferably 2-ethylhexyl, or n-octyl, pharmaceutical compositions are preferred, forming in aqueous media lipid-based drug delivery systems (DDS). Preferred DDS are mentioned below.

In a further embodiment of the present invention R³ of a compound according to formula I is (C₀-C₈)-alkylene-(C₃-C₁₄)-cycloalkyl, (C₀-C₈)-alkylene-(3-14-membered)-heterocycloalkyl, (C₁-C₈)-alkylene-(C₃-C₁₄)-cycloalkenyl, (C₀-C₈)-alkylene-(3-14-membered)-heterocycloalkenyl, (C₀-C₈)-alkylene-(C₆-C₁₄)-aryl or (C₀-C₈)-alkylene-(5-14-membered)-heteroaryl.

Further, one or more non-adjacent groups (CR⁴R⁵), preferably one group (CR⁴R⁵), may be replaced by CO, O, OCO, COO, CON(R¹⁹), N(R²⁰)CO, preferably CO. In a preferred embodiment of the present invention the group (CR⁴R⁵) which is directly bound to the oxygen atom of the group of formula II is not replaced by O, CO, OCO, COO, CON(R¹⁹), N(R²⁰)CO, preferably, the group (CR⁴R⁵) which is directly bound to R⁶ is also not replaced by O, CO, OCO, COO, CON(R¹⁹), N(R²⁰)CO.

In a further embodiment the present invention relates to a pharmaceutical composition comprising one or more compounds of formula I or a salt thereof and one or more pharmaceutically acceptable carriers, wherein the residues are defined as mentioned herein, wherein in the case that R² is H, R³ in the residue R¹ is not tert-butyl.

In a further embodiment the present invention relates to a pharmaceutical compound according to formula I or a salt thereof wherein the residues are defined as mentioned herein, wherein in the case that R² is H, R³ in the residue R¹ is not tert-butyl.

The residues and indices R⁴, R⁵, R⁶ and x mentioned in R³ of a compound according to formula I preferably have independently of one another the following meanings:

Each R⁴ and R⁵ of a compound according to formula I are independently of one another selected from the group consisting of H, (C₁-C₁₅)-alkyl, (C₁-C₁₅)-alkylene-hydroxy, (C₀-C₁₅)-alkylene-(C₁-C₁₅)-alkoxy, OH, (C₀-C₁₅)-alkylene-N(R⁷)CO—(C₁-C₁₅)-alkyl, (C₀-C₁₅)-alkylene-CON(R⁸)(R⁹), (C₀-C₁₅)-alkylene-COO—(C₁-C₁₅)-alkyl, (C₀-C₁₅)-alkylene-N(R¹⁰)(R¹¹), SO₃R¹⁷, (C₀-C₁₅)-alkylene-(C₆-C₁₈)-aryl, or (C₀-C₁₅)-alkylene-(5-18-membered)-heteroaryl,

preferably, each R⁴ and R⁵ of a compound according to formula I are independently of one another selected from the group consisting of H, (C₁-C₈)-alkyl, (C₁-C₈)-alkylene-hydroxy, (C₀-C₈)-alkylene-(C₁-C₄)-alkoxy, OH, (C₀-C₈)-alkylene-N(R⁷)CO—(C₁-C₈)-alkyl, (C₀-C₈)-alkylene-CO—N(R⁸)(R⁹), (C₀-C₈)-alkylene-COO—(C₁-C₈)-alkyl, (C₀-C₈)-alkylene-N(R¹⁰)(R¹¹), (C₀-C₈)-alkylene-(C₆-C₁₄)-aryl, SO₃R¹⁷, preferably H, (C₁-C₄)-alkyl, and (C₀-C₂)-alkylene-N(R⁷)CO—(C₁-C₄)-alkyl,
more preferably, each R⁴ and R⁵ of a compound according to formula I are independently of one another selected from the group consisting of H, (C₁-C₄)-alkyl, and (C₀-C₂)-alkylene-N(R⁷)CO—(C₁-C₄)-alkyl;
or

### R⁴ and R⁵ of the same group (CR⁴R⁵) or R⁴ and R⁵ of different groups (CR⁴R⁵) may form together a carbocyclic or heterocyclic ring having from 3 to 6 atoms,

additionally, one or more non adjacent groups (CR⁴R⁵) may be replaced by O, CO, OCO, COO, CON(R¹⁹), N(R²⁰)CO, or NR²¹, preferably CO,
R⁶ of a compound according to formula I is in a preferred embodiment independently H, (C₁-C₁₅)-alkyl, (C₂-C₂₀)-alkenyl, (C₂-C₁₅)-alkynyl, OH, O—(C₁-C₈)-alkyl, O—(C₀-C₈)-alkylene-(C₆-C₁₄)-aryl, CO—O—(C₁-C₈)-alkyl, CO—N(R¹²)(R¹³), N(R¹⁴)CO—(C₁-C₈)-alkyl, N(R¹⁵)(R¹⁶), SO₃R¹⁸, (C₀-C₁₅)-alkylene-(5-18-membered)-heteroaryl, or (C₀-C₁₅)-alkylene-(C₆-C₁₈)-aryl, more preferably H, OH, O—(C₁-C₈)-alkyl, O—(C₀-C₈)-alkylene-(C₆-C₁₄)-aryl, CO—O—(C₁-C₈)-alkyl, CO—N(R¹²)(R¹³), N(R¹⁴)CO—(C₁-C₈)-alkyl, N(R¹⁵)(R¹⁶), SO₃R¹⁸ most preferably O—(C₁-C₈)-alkyl, CO—O—(C₁-C₄)-alkyl, CO—N(R¹²)(R¹³), N(R¹⁴)CO—(C₁-C₄)-alkyl, or N(R¹⁵)(R¹⁶), most preferably O—(C₁-C₈)-alkyl, CO—O—(C₁-C₄)-alkyl, CO—N(R¹²)(R¹³), N(R¹⁴)CO—(C₁-C₄)-alkyl, or N(R¹⁵)(R¹⁶), still more most preferably CO—O—(C₁-C₄)-alkyl or CO—N(R¹²)(R¹³).

x of a compound according to formula I is preferably 1 to 8, more preferably 1 to 4.

In one embodiment (CR⁴R⁵)ₓ is (C₁-C₄)-alkylene, preferably (C₁-C₂)-alkylene.

The present invention further relates to pharmaceutical compositions as described above, wherein in the compound of formula I
each R³ is independently (C₁-C₁₀)-alkyl, (CR⁴R⁵)ₓ—R⁶, (C₃-C₂₀)-alkenyl, (C₃-C₈)-alkynyl, (C₀-C₈)-alkylene-(C₃-C₁₄)-cycloalkyl, (C₀-C₈)-alkylene-(3-14-membered)-heterocycloalkyl, (C₀-C₈)-alkylene-(C₆-C₁₄)-aryl, or (C₀-C₈)-alkylene-(5-14-membered)-heteroaryl,
   wherein the total carbon number of R³ is at most 15,
(CR⁴R⁵)ₓ is (C₁-C₄)-alkylene, preferably (C₁-C₂)-alkylene, and
R⁶ is independently CO—O—(C₁-C₄)-alkyl or CO—N(R¹²)(R¹³).

The residues R⁷, R¹⁴, R¹⁷, R¹⁸, R¹⁹, R²⁰ and R²¹ of a compound according to formula I mentioned in R⁴ and R⁵ respectively in R⁶ are independently of one another H, (C₁-C₈)-alkyl, more preferably H, (C₁-C₄)-alkyl.

The residues R⁸, R⁹, R¹⁰, R¹¹, R¹², R¹³, R¹⁵, and R¹⁶ of a compound according to formula I mentioned in R⁴ and R⁵ respectively in R⁶ are preferably independently of one another, H, (C₁-C₈)-alkyl, more preferably (C₁-C₄)-alkyl.

The residue Y of a compound according to formula I is sulfur or oxygen, preferably oxygen.

The residues R²² and R²³ of a compound according to formula I are independently selected from the group consisting of C₁-C₁₅ alkyl, C₁-C₈ alkyl, or C₁-C₄ alkyl. In a non-limiting embodiment, R²² and R²³ are ethyl.

Most preferred entacapone carbonates are entacapone carbonates of formula I, wherein R² is H and R¹ is a group of formula II. The compounds according to this preferred embodiment can be represented by the structures:

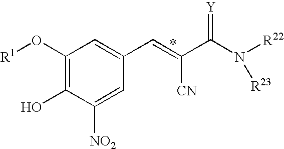

or in an even more preferred embodiment:

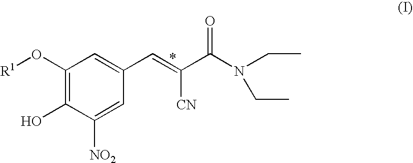

### One skilled in the art would understand that additional/alternative subsets of compounds of formula I can be derived by combining two or more of the above embodiments.

It has been found that the pharmaceutical compositions of the present invention comprising the entacapone carbonates of formula I are suitable to provide reproducible and constant plasma levels.

In one embodiment the present invention relates to pharmaceutical compositions comprising entacapone carbonates as shown in table 1.

The entacapone carbonates of formula I of the present invention are suitable prodrugs of entacapone. According to the present application a prodrug of entacapone is an entacapone derivative which is metabolized into entacapone.

It has been found by the inventors that a carbonate group in m-position to the NO₂ group of the entacapone is suitable to improve the bioavailability of entacapone. Further, by introducing a carbonate group in the m-position of the NO₂ group the glucuronidation of entacapone and therefore the elimination of the glucuronide may be delayed.

The entacapone carbonates of the present invention may be prepared by any process known by a person skilled in the art. In various embodiments, prodrugs described herein are synthesized by reacting a hydroxyl intermediate with reagents that result in incorporation of carbonate ester groups at one or both hydroxyls of the nitro-substituted phenyl rign of the intermediate. Several general methods, suitable for use with both the propeneamide and thioamide starting materials, are given below.

In preferred embodiments of the present invention the entacapone carbonates of formula I are prepared starting from entacapone by three different general methods (A, B and C).

### Method A

According to method A a cyclic ester is formed by reaction of entacapone with phosgene (step (a)), followed by selective ring-opening via alcoholysis (step (b)). It has been found by the inventors that method A is suitable for selectively preparing monocarbonates of entacapone, wherein R² is H.

The reaction is shown in the scheme below:

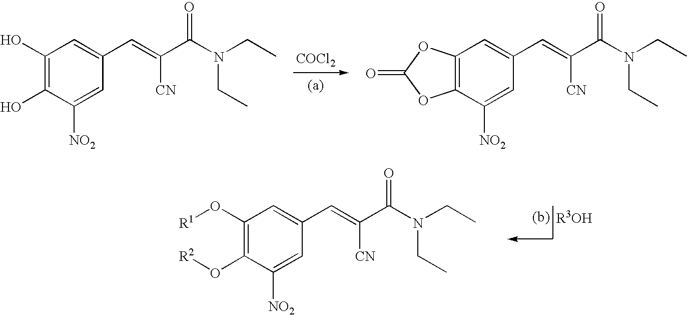

wherein R¹ and R² and R³ are defined as mentioned before.

### Step (a)

The reaction (step a)) is usually carried out in tetrahydrofuran (THF) or a hydrocarbon solvent, such as for example in toluene.

Further, preferably a base, for example pyridine, is added to the reaction mixture.

In the first step (a) of the reaction, entacapone is reacted with phosgene. The reaction is usually carried out at temperatures from −15° C. to +30° C., preferably from −10° C. to ambient temperature. The reaction time of step (a) is in general 0.5 to 5 h, preferably 2 to 4 h, more preferably 3 to 4 h.

Usually a precipitate is formed which is preferably removed by filtration. The filtercake obtained is usually washed with the hydrocarbon solvent used in the reaction, for example toluene or THF. The filtrate obtained comprising the cyclic ester is evaporated, preferably under vacuum, and the cyclic ester is obtained as a solid.

### Step (b)

The solid cyclic ester obtained is usually taken up in an organic solvent, preferably selected from the group consisting of dichloromethane and THF. To the reaction mixture obtained the appropriate alcohol R³OH is added. That alcohol is typically commercially available or, alternatively, can be prepared by a method known by one skilled in the art. The alcohol is typically added at ambient temperature. The alcohol is usually added in at least equimolar amounts, preferably in a molar excess, more preferably in a 2 to 3 fold molar excess, in relation to the cyclic ester obtained in step (a). The reaction mixture is then usually agitated for 8 to 30 h, preferably 8 to 24 h. The agitation is usually carried out at ambient temperature.

Preferably, inprocess controls were carried out, for example by thin layer chromatography (TLC) or high performance liquid chromatography (HPLC) to watch the reaction progress and to determine the completion of the reaction. After completion of the reaction the reaction mixture is worked up by a process known by a person skilled in the art. Usually, the reaction mixture is washed one or more times, for example twice, with an acid, preferably with hydrochloric acid, more preferably with 2N HCl, and with water. The organic layer obtained is usually dried, for example over sodium sulphate or other suitable drying agents known by a person skilled in the art, and evaporated under vacuum, whereby the desired carbonate is obtained as a solid or as an oil, which in most cases solidifies on standing.

If desired, the crude product obtained is purified by suitable purification methods known by a person skilled in the art, for example by recrystallization, preferably by recrystallization with hexane, ethyl acetate, or by chromatography, preferably flash chromatography.

The crude product is usually obtained in yields in general above 50%, preferably above 60%, more preferably of at least 70%, most preferably in yields from 70 to 80%. If desired, the obtained crude product is purified by suitable purification methods known by one skilled in the art, for example, by recrystallization, preferably by recrystallization with hexane or ethyl acetate, or by chromatography, preferably flash chromatography. The purified product is obtained with purities of at least 95%, whereby the yield of the purified product is in general well above 10%. The purity and identity of the desired product is usually determined by HPLC/electrospray ionization-mass spectrometry (HPLC/ESI-MS)

### Method B

According to method B the desired carbonate of formula I is prepared by reaction of entacapone with chloroformic acid esters ClCO₂R³, wherein R³ is as defined before.

The reaction is shown in the scheme below:

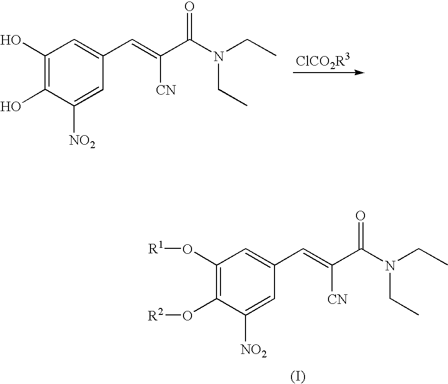

wherein R¹, R² and R³ are defined as mentioned before.

Usually, monosubstituted carbonate derivatives, wherein R¹ is a residue of formula II and R² is H are prepared. However, the preparation of disubstituted carbonated derivatives of entacapone is also possible by carrying out method B. In some cases, a disubstituted derivative is obtained as a by-product when the monosubstituted derivative is prepared. This disubstituted product may be separated for example by flash chromatography.

In a preferred embodiment of the present invention the reaction is carried out as described in the following:

Entacapone is dissolved in water and a base, preferably an alkali carbonate, e.g. sodium or potassium carbonate, is added. Further, the appropriate chloroformic acid ester (ClCO₂R³) is added under inert gas atmosphere, preferably under nitrogen atmosphere. The appropriate ester ClCO₂R³ is typically commercially available or, alternatively, can be prepared by a method known by one skilled in the art. The chloroformic acid ester is usually added in an excess in relation to entacapone. Preferably a molar excess of the chloroformic acid ester in relation to entacapone of about 10 to 20%, more preferably of about 20% is used. The addition of the chloroformic acid ester is usually carried out at ambient temperature. In a preferred embodiment the chloroformic acid ester is added drop-wise, for example with a syringe. After addition of the chloroformic acid ester the reaction mixture is agitated, preferably at ambient temperature, and the progress of the reaction is watched and the end of the reaction is determined, usually by TLC. The agitation is usually carried out for 8 to 30 h, preferably 8 to 24 h. After completion of the reaction the reaction mixture is usually worked up as known by a person skilled in the art. In a preferred embodiment the reaction mixture is extracted one or more times, preferably twice, with ethyl acetate and then dried over sodium sulphate or another suitable drying agent known by a person skilled in the art. The solvent of the reaction mixture is then removed under vacuum, whereby the desired product is obtained as crude product, in general as an oil which solidifies on standing.

Further, the crude product is usually purified by a process known by a person skilled in the art. Usually, the purification is carried out by column chromatography. The crude product is in general obtained in yields over 60%, preferably of at least 70%, more preferably between 70 and 80%. The purified product is obtained in a purity of at least 95%. The yield of the purified product is in general well above 10%. The purity and identity of the desired product is usually determined by HPLC/ESI-MS.

### Method C

A third synthesis of carbonate esters from starting hydroxyl materials is reaction with pyrocarbonates as illustrated for entacapone in the scheme

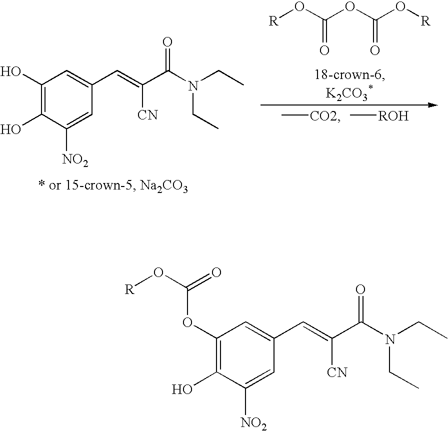

Starting hydroxyl material (1 equivalent), K₂CO₃ or Na₂CO₃ (2 equivalents) and a catalytic amount of crown ether (ca. 1-5 mole %) are dissolved in a suitable solvent such as THF and stirred, for example at 50° C. overnight. After cooling down to ambient temperature, the mixture is filtered and the solvent evaporated under vacuum to afford the crude product with typical yields above 80%. Purification is carried out by flash chromatography or by recrystallization from suitable solvents such as petroleum ether-ethyl acetate, or hexanes-ethyl acetate. Dicarbonate esters can be synthesized by including two equivalents of starting hydroxyl material; alternatively, the isolated monoester can be subject to reaction with a second equivalent of pyrocarbonate.

Pyrocarbonates used are either commercially available or can be prepared by literature procedures. For example di-3-pentyldicarbonate is prepared following the procedure described for di-sec-butyldicarbonate in Chem. Eur. J., 2000; 6; No. 21 page 3988.

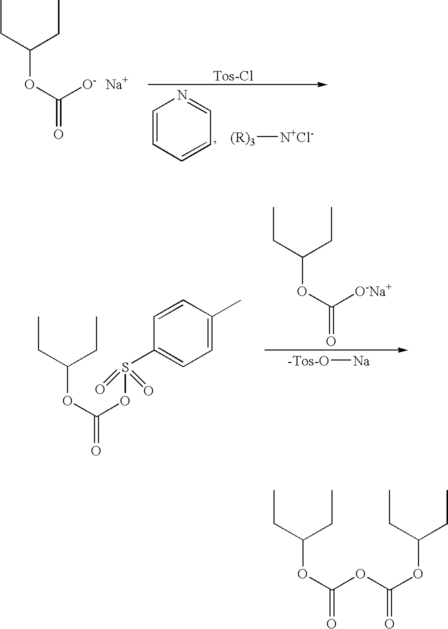

The entacapone used as starting material in the preparation of the entacapone carbonates of the present invention is commercially available or may be prepared by methods known in the art, for example by Knoevenagel condensation of 3,4-dihydroxy-5-nitrobenzaldehyde with N,N-diethyl-2-cyanoacetamide. Preferably, the E-isomer of entacapone is used in the preparation of the entacapone carbonates.

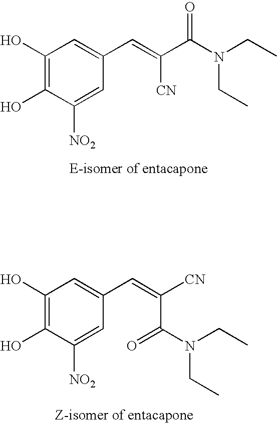

Suitable methods for the preparation of entacapone are for example described in GB 2,200,109 A, EP 0 426 468 A2, WO 2005/070881 A1, WO 2005/063696 A2, WO 2005/063695 A1 and WO 2005/063693 A1.

GB 2,200,109 A discloses the preparation of entacapone by reaction of 3,4-dihydroxy-5-nitrobenzaldehyde and N,N-hydrochloric diethylcyanoacetamide in ethanol in the presence of catalytic amounts of piperidine acetate. However, according to EP 0 426 468 A2, entacapone is obtained in the process according to GB 2,200,109 in form of a mixture of two geometric isomers, E- and Z-isomer (70-80% E-isomer and 30-20% Z-isomer). Additionally, there exist—according to EP 0 426 468 A2—at least two polymorphic forms A and B of entacapone. It has been found by the inventors of EP 0 426 468 A2 that essentially pure and stable polymorphic form A of the E-isomer is obtained when the crude product obtained by the process as disclosed in GB 2,200,109 A is recrystallized from formic acid or acetic acid with a catalytic amount of hydrochloric acid or hydrobromic acid added.

WO 2005/063695 A1 and WO 2005/063696 A2 disclose novel crystalline forms—C, D and according to WO 2005/063696 A2 additionally E—of entacapone and the production thereof. Further, WO 2005/063695 A1 and WO 2005/063696 A2 disclose improvements of the Knoevenagel condensation of 3,4-dihydroxy-5-nitrobenzaldehyde and N,N-diethylcyanoacetamide. Instead of piperidine/acetic acid diethylamine/acetic acid is used as catalyst in the Knoevenagel condensation according to WO 2005/063695 A1 and WO 2005/063696 A2 to avoid the formation of by-products. Further, N,N-diethyl-2-cyanoacetamide is prepared by reaction of cyanoacetic acid and diethylamine in the presence of dicyclohexyl carbodiimide to avoid low yields and expensive starting materials. Finally, the demethylation of commercially available 5-nitrovanilline to 3,4-dihydroxy-5-nitrobenzaldehyde is carried out in the presence of AlCl3/pyridine in chlorobenzene instead of hydrobromic acid, because the 3,4-dihydroxy-5-nitrobenzaldehyde is obtained in high yield and may be used as starting material in the Knoevenagel condensation without further purification.

WO 2005/070881 A1 discloses an improved process for the manufacture of the E-isomer of entacapone in its polymorphic form A, by Knoevenagel condensation of 3,4-dihydroxy-5-nitrobenzaldehyde and N,N-diethylcyanoacetamide in the presence of a base in alcoholic solution and extraction of the reaction mixture obtained comprising crude entacapone as a mixture of E- and Z-isomers by pouring the crude reaction mixture into an aqueous ethyl acetate solution followed by adjusting the pH between 3.5 and 4.0, preferably with acetic acid. By the extraction process the E-isomer of entacapone form A is obtained in 99.7% purity (HPLC).

WO 2005/063693 A1 discloses an improved process for the preparation of entacapone which comprises: (i) reacting 3-alkoxy-4-hydroxy-5-nitrobenzaldehyde with N,N-diethylaminocyanoacetamide in the presence of a mild acid catalyst and a solvent at a temperature in the range of 50 to 115° C., to get intermediates of entacapone, wherein the OH-group in 3-position is replaced by OR (R=methyl or ethyl); (ii) treating the 3-O-alkylated entacapone with acid catalysts in the presence of an organic base and solvents at a temperature in the range of 20 to 60° C. to get the crude entacapone and if desired, (iii) purifying the crude entacapone obtained using a solvent or a solvent mixture. According to WO 2005/063693 A1 entacapone is obtained by the process mentioned before in high yields and a storage stable intermediate of entacapone is presented. The separation of the E-isomer of entacapone from the reaction mixture obtained in the process according to WO 2005/063693 A1 is not mentioned.

As mentioned before, the E-isomer of entacapone is the preferred starting material for the preparation of the entacapone carbonates of the present invention, because the Z-isomer has been shown to be unstable under the influence of heat or acids as mentioned in EP 0 426 468 A2 and WO 2005/070881 A1.

In the state of the art mentioned before, different methods are presented to obtain the E-isomer of entacapone in high purity. However, all methods presented in the state of the art have the drawback that an additional step (recrystallization or extraction) is necessary to isolate the E-isomer from a crude reaction mixture comprising the E-isomer and the Z-isomer of entacapone. It is desirable to obtain the E-isomer of entacapone directly from the reaction mixture to avoid expensive additional steps and loss of yield of E-entacapone by recrystallization or extraction.

A new process for the preparation of entacapone has been found by the inventors, wherein the E-isomer of entacapone is predominantly obtained directly from the reaction mixture. The new process comprises the following step:
(ii) Reaction of an aldehyde of formula III with N,N-diethylcyanoacetamide (IV) in the presence of ammonium acetate, wherein the E-isomer of entacapone (V) or an intermediate of formula Va is obtained,

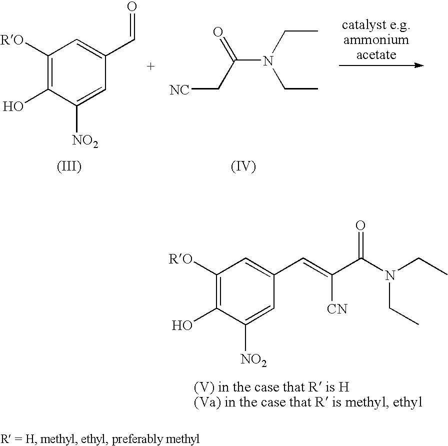

wherein a catalyst such as ammonium acetate, piperidine or β-alanine, preferably ammonium acetate is employed in a molar excess in relation to the aldehyde of formula III.

The aldehyde of formula III is preferably 5-nitrovanillin (R′=methyl), which is commercially available. 3,4-dihydroxy-5-nitrobenzaldehyde (R′═H) and 3-ethoxy-4-hydroxy-5-nitrobenzaldehyde which may alternatively be used are prepared by methods known in the art (see for example the documents mentioned above).

N,N-diethylcyanoacetamide (IV) may be prepared by methods known in the art (see for example the documents mentioned above). In a preferred embodiment N,N-diethylcyanoacetamide (IV) is prepared by deprotonation of diethylamine, for example with a lithium base like n-hexyllithium, followed by reaction with ethylcyanoacetate. Suitable reaction conditions are known by a person skilled in the art.

The molar ratio of the aldehyde of formula III and N,N-diethylcyanoacetamide (IV) is not critical and is usually about 1:2 to 2:1. Preferably, N,N-diethylcyanoacetamide (IV) is employed in a molar excess of up to 15%, preferably 10% in relation to the aldehyde of formula III.

The reaction is carried out in the presence of ammonium acetate. It has been found by the inventors, that the E-isomer of entacapone is obtained, when ammonium acetate is employed in a molar excess in relation to the aldehyde of formula III. Preferably, an at least 1.5 molar excess is used, more preferably an at least 2 molar excess, even more preferably an at least 2.2 molar excess is used. The ammonium acetate decomposes during the reaction. The acetic acid formed during this process is responsible for preferred formation of the E-isomer. The Knoevenagel condensations works as well in the presence of other commonly known catalysts (such as piperidine and β-alanine).

The reaction of the aldehyde of formula III and N,N-diethylcyanoacetamide (IV) is usually carried out in a solvent. Suitable solvents are alcohols, for example ethanol.

The reaction temperature is usually from 25° C. to 150° C., preferably from 40° C. to 100° C. If ethanol is used as solvent, the reaction temperature is usually the reflux temperature of ethanol (78° C.).

In a preferred embodiment the reaction is carried out by providing N,N-diethylcyanoacetamide (IV) in the solvent used in the reaction and adding the aldehyde of formula III and acetic acid or its salt at ambient temperature. The reaction mixture is agitated and heated to the temperature mentioned before, preferably to reflux, if ethanol is used as solvent. The progress of the reaction is usually observed by in-process control. The reaction is stopped when most of the aldehyde of formula III, preferably at least 90% by weight, is consumed. The reaction mixture is then cooled, for example to about −5° C. or lower temperatures and agitated at said temperature, for example for about 1 hour. The reaction mixture is worked up as known by a person skilled in the art. Preferably, the precipitate obtained is collected usually in a funnel and washed with the solvent used in the reaction, which is cooled to about −10° C. or lower. The solid obtained is dried by a method known in the art.

In the case that 3,4-dihydroxy-5-nitrobenzaldehyde (R′═H) is used as starting material, the product obtained is the E-isomer of entacapone (V). In the case that 5-nitrovanillin (R′=methyl) or 3-ethoxy-4-hydroxy-5-nitrobenzaldehyde (′R=ethyl) is used as starting material, not the E-isomer of entacapone, but an intermediate of entacapone carrying in the 3-position of entacapone instead of the OH group a methoxy or ethoxy group (Va) is obtained. Said intermediate (Va) may be converted to the E-isomer of entacapone (V) by any process known in the art. Preferably, the E-isomer of entacapone (V) is obtained from the intermediate (Va) by the following step:
(iii) Reaction of the intermediate of formula Va with AlCl₃ and a base, preferably pyridine, in a solvent whereby the E-isomer of entacapone (V) is obtained

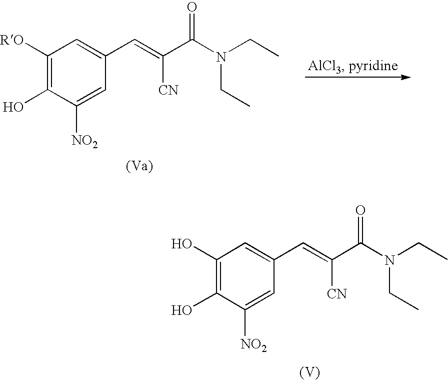

R′=methyl, ethyl, preferably methyl

The sovent used in step iii) is usually chloroform. The intermediate of formula Va and AlCl₃ are usually employed in a molar ratio of 0.7:1 to 1:0.7, preferably in a molar ratio of 1:1 to 1:1.1. The pyridine is usually used in a molar excess in relation to the intermediate of formula Va, preferably in a 4-fold molar excess.

The reaction is preferably carried out by providing the solvent, the intermediate of formula Va and AlCl₃ and agitating and cooling the reaction mixture to a temperature of ≦0° C., for example 0° C. to −5° C. Pyridine, usually dry pyridine dissolved in the reaction solvent, is added slowly, preferably dropwise, to the reaction mixture. After addition of pyridine the reaction mixture is heated under agitation to 30° C. to 80° C., preferably 40° C. to 70° C., more preferably to reflux until the cleavage to entacapone is complete. The reaction mixture is then worked up as known by a person skilled in the art.

In a preferred embodiment of the present invention the E-isomer of entacapone which is preferably used in the process for the preparation of the entacapone carbonates of the present invention is prepared by the following process:
(i) Preparation of N,N-diethylcyanoacetamide (IV) by deprotonation of diethylamine with a lithium base followed by reaction with ethylcyanoacetate

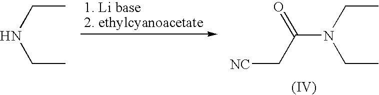

(ii) Reaction of 5-nitrovanillin with N,N-diethylcyanoacetamide (IV) in the presence of ammonium acetate, wherein an intermediate of formula Va is obtained,

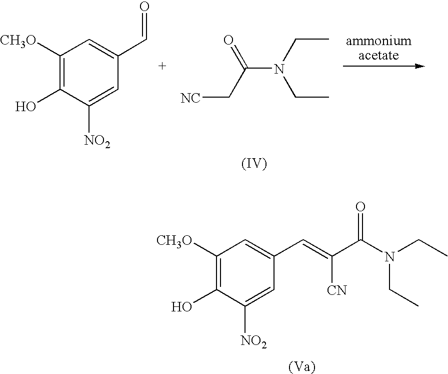

wherein ammonium acetate is employed in a molar excess in relation to the 5-nitrovanillin;
(iii) Reaction of the intermediate of formula Va with AlCl₃ and pyridine in chloroform whereby the E-isomer of entacapone (V) is obtained

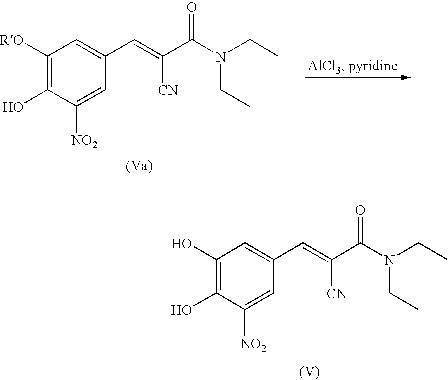

R′=methyl, ethyl, preferably methyl

The reaction conditions and preferred embodiments of the reaction steps (i), (ii) and (iii) are mentioned before.

The E-isomer of entacapone (V) is obtained by the process of the present invention in an overall yield of at least 55% and usually has a purity of >98% (determined by HPLC). An additional recrystallization or extraction step for obtaining the E-isomer of entacapone is not necessary.

Entacapone derivatives containing R²² and R²³ other than diethyl are synthesized by analogous methods. To illustrate, an amine R²²NHR²³ is reacted with a lithium base and then further reacted with cyanoacetate. The reaction product is then reacted with aldehyde III as described above. In this way a wide variety of entacapone derivatives and their carbonate ester prodrugs are readily synthesized.

Thioamides analogous to structure V, which are precursors of the carbonate compounds described above, are prepared in similar fashion from aldehyde III and N,N-dialkylcyanothioacetamide. The latter starting material is prepared by N,N-dialkylation of cyanothioacetamide or by reaction of a lithium amide with a cyanothioacetate ester in a reaction analogous to that shown immediately above for the cyanoacetamide. Alternatively, the thioamide analogous to structure V (or a thioamide as defined according to formula I) can be formed from entacapone (or from a compound according to formula I wherein Y is oxygen) by using a sulfurization agents such as e.g. Lawesson's reagent or P2S5.

In a further embodiment the present invention relates to entacapone preparable by the process of the present invention.

The pharmaceutical composition of the present invention comprises beside the entacapone carbonate of formula I one or more pharmaceutically acceptable carriers.

Suitable pharmaceutically acceptable carriers depend on the pharmaceutical form and are known by a person skilled in the art.

As used herein, “pharmaceutically acceptable carriers” includes any and all solvents and solvent mixtures, dispersion media, complexation agents, surface active excipients, solid carriers, coatings, antibacterial and antifungal agents, isotonic and absorption delaying agents for pharmaceutically active substances and mixtures thereof, as well known in the art.

Examples for pharmaceutically acceptable carriers are selected from the group consisting of gelatin, lactose, sugar alcohols, e.g. mannitol, starch, e.g. corn starch, magnesium stearate, talc, vegetable oil, microcrystalline cellulose, polysorbate, sodium lauryl sulphate, colloidal silicon oxide, copolyvidone, water, buffered aqueous solutions, ethanol, polyalkylene glycols, preferably polyethylene glycols, e.g. PEG 400, propylene glycol, Tween® 80 (i.e. PEG (20) sorbitol monooleate), DMSO, mixtures of water and cosolvents, e.g. aqueous solutions comprising alcohols like ethanol and/or polyalkylene glycols like polyethylene glyol, complexation agents like cyclodextrins, e.g. α-cyclodextrin, (α-CD) or hydroxypropyl-β-cyclodextrin (HP-β-CD), surfactants like anionic, cationic, non-ionic and amphoteric surfactants, salts of bile acids or lipids, e.g. animal or vegetable phospholipids, esters of polyols like glycerol and/or polyethylene glycol with fatty acids, micelle-forming agents, and oils like corn oil, or mixtures of two or more of the components mentioned before.

In one embodiment of the present invention the pharmaceutically acceptable carrier is a phosphate buffer aqueous system without further components.

Further suitable pharmaceutically acceptable carriers as well as suitable additives which may be used in the compositions of the present invention are mentioned below.

In one embodiment the present invention relates to pharmaceutical compositions of the present invention forming in aqueous media lipid-based drug delivery systems (DDS). Preferably, said pharmaceutical compositions comprise at least one surfactant beside the at least one entacapone carbonate of formula I or salt thereof. Suitable surfactants are mentioned above. The lipid-based drug delivery systems may form the following structures:
micelles, microemulsions, emulsions (i.e. simple self-assembly structures of lipids and surfactants)
liposomes (i.e. dispersed closed bilayer assembleys of a lamellar phase in water)
nanoparticles of non-lamellar phases (e.g. cubic, hexagonal, sponge).

Preferably, the lipid-based drug delivery systems form micelles, microemulsions or emulsions. The HLB-value (hydrophile-lipophile-balance) of suitable surfactants or surfactant mixtures for the formation of micelles, microemulsions or emulsions is in general of from 8 to 18, preferably 10 to 18, more preferably 12 to 16. More preferably, the lipid-based drug delivery systems form an SEDDS (self-emulsifying drug delivery system) or an SMEDDS (self-microemulsifying drug delivery system). SEDDS and SMEDDS are mixtures, ideally isotropic, of oil(s) (i.e. lipid(s), e.g. lipophilic carbonates of formula I or salts thereof), at least one surfactant, optionally at least one co-surfactant and optionally at least one co-solvent, which emulsify spontaneously to produce fine oil-in-water emulsions when introduced into an aqueous phase under gentle agitation. The gentle agitation may be for example provided by gastric mobility.

Suitable pharmaceutically acceptable carriers for forming, optionally together with further additives, an SEDDS or an SMEDDS are for example pharmaceutically acceptable carriers comprising ethoxylated surfactants or other surfactants having an HLB value as mentioned above, and optionally alcohols or polyols, for example pharmaceutically acceptable carriers comprising a combination of phospholipids and/or lecithins and aqueous solutions of polyols or carbohydrates as for example disclosed in WO 2004/047791. Further suitable examples are pharmaceutically acceptable carriers comprising micelle-forming agents, for example non-ionic solubilizers or emulsifying agents having a hydrophilic part and a hydrophobic part, for example an emulsifying agent, wherein the hydrophobic part is glycerol polyethylene glycol oxystearate together with fatty acid glycerol polyglycol esters and the hydrophilic part are polyethylene glycols and glycerol ethoxylate, like Cremophor® RH 40, further suitable examples are mixtures of animal or vegetable phospholipids and/or lecithins with polyols or carbohydrates, e.g. NanoSolve® 5401 (NanoSolve is a phospholipid product of Lipoid GmbH, which mainly comprises lecithin and further glycerol), esters of glycerol and PEG with fatty acids, e.g. Labrasol®, Vitamin E derivatives e.g. TPGS (α-tocopheryl polyethylene glycol 1000 succinate), mixtures of Vitamin E derivatives, e.g. TPGS, with propylene glycol (PG) (TPGS/PG), preferably in a weight ratio of 25% by weight of TPGS and 75% by weight of PG. Most preferred are esters of glycerol and PEG with fatty acids, especially Labrasol®, and mixtures of Vitamin E derivatives, e.g. TPGS with propylene glycol (PG), preferably in a weight ratio of 25% by weight of TPGS and 75% by weight of PG. Most preferred are esters of glycerol and PEG with fatty acids, especially Labrasol®.

The pharmaceutical compositions may comprise further excipients and/or additives. Suitable further excipients and/or additives are mentioned before and below.

The compounds of formula I may be administered in a convenient manner, such as by oral, intravenous, intramuscular, intrathecal or subcutaneous routes. Oral administration is preferred.

The compound of formula I may be orally administered, for example, with an inert diluent or with an assimilable edible carrier or it may be enclosed in capsules, or it may be compressed into tablets, or it may be incorporated directly into the food of the diet. For oral therapeutic administration, the active compound of formula I may be incorporated with excipients and used in the form of ingestible tablets, buccal tablets, troches, capsules, elixirs, suspensions, syrups, wafers, pills, soft gel caps, powders, solutions, dispersions, liquids and the like. Such compositions and preparations should contain at least 1% of active compound of Formula I. The percentage of the compositions and preparations may, of course, be varied and may conveniently be between about 5 to about 80% of the weight of the unit.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or otherwise modify the physical form of the dosage unit. For instance, tablets, pills, or capsules may be coated with shellac, sugar or both. A syrup or elixir may contain the compound of formula I, sucrose as a sweetening agent, methyl and propylparabens as preservatives, a dye and flavouring such as cherry or orange flavour.

Of course, any material used in preparing any dosage unit form should be pharmaceutically pure and substantially non-toxic in the amounts employed.

In one embodiment of the invention the compound of formula I is included in a capsule. The capsule can be a hard or soft shell capsule. The capsule can be made from any suitable film forming material comprising e.g. gelatin, cellulose derivatives, pullulan or other glucans, polyvinyl alcohol, pectin, modified starches, such as starch ethers and oxidized starch, more particularly hydroxyethylated starch (HES) or hydroxypropylated starch (HPS)—alone or mixtures thereof and if appropriate in a mixture with a setting system or further components. The cellulose derivatives used for the manufacture of capsules include, but are not limited to, hydroxypropyl cellulose, hydroxyethyl cellulose, hydroxypropyl methyl cellulose, hydroxymethyl cellulose, methylcellulose, ethyl cellulose, cellulose acetate, cellulose acetate phthalate, cellulose acetate trimelliate, hydroxypropylmethyl cellulose phthalate, hydroxypropylmethyl cellulose succinate, carboxymethyl cellulose sodium, and mixtures thereof. Preferred cellulose derivatives are hydroxypropyl cellulose, hydroxyethyl cellulose, hydroxypropyl methyl cellulose, hydroxymethyl cellulose, methylcellulose, and ethyl cellulose.

In addition, the compound of formula I may be incorporated into sustained-release preparations and formulations (retard compositions). For example, sustained release dosage forms are contemplated wherein the compound of formula I is bound to an ion exchange resin which, optionally, can be coated with a diffusion barrier coating to modify the release properties of the resin.

The compound of formula I may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersions and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the compound of formula I in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique.

It is especially advantageous to formulate the pharmaceutical compositions of the present invention in dosage unit form for ease of administration and uniformity of dosage. Dosage unit form as used herein refers to physically discrete units suited as unitary dosages for the mammalian subjects to be treated; each unit containing a predetermined quantity of the compound of formula I calculated to produce the desired therapeutic effect in association with the required pharmaceutical carrier. The specifics for the novel dosage unit forms of the invention are dictated by and directly dependent on (a) the unique characteristics of the compound of formula I and the particular therapeutic effect to be achieved, and (b) the limitations inherent in the art of compounding the compound of formula I for the treatment of diseases in patients having a disease condition in which bodily health is impaired.

The compound of formula I is compounded for convenient and effective administration in effective amounts with a suitable pharmaceutically acceptable carrier and optionally further suitable additives and excipients in dosage unit form as hereinbefore described. The dosage of the compound of formula I is depending on the way of administration, age and weight of the patient, kind and severeness of the disease to be treated, etc. The daily dosage calculated as entacapone is in general from 10 to 2000 mg/d, for example 200 to 2000 mg/d, 800 to 1800 mg/d, or from 100 to 1600 mg/d. The daily dose may be administered in one single dosage unit per day or in two or more dosage units per day.

The compound of formula I of the present invention is in general administered in combination with L-dopa and preferably also a decarboxylase inhibitor such as carbidopa or benseracid. Carbidopa and benseracid are commercially available and known by a person skilled in the art.

The compound of formula I and L-dopa and optionally the decarboxylase inhibitor may be administered together, i.e. in one single dosage form comprising the compound of formula I, L-dopa and optionally the decarboxylase inhibitor, or may be administered separately, i.e. in separate dosage forms, one dosage form comprising the compound of formula I and one further dosage form comprising L-dopa and optionally the decarboxylase inhibitor.

In a further embodiment the pharmaceutical compositions of the present invention additionally comprise L-dopa and preferably also a decarboxylase inhibitor such as carbidopa or benseracid.

Beside the pharmaceutical compositions as defined before, the present invention also relates to compounds of formula (I) or a salt thereof

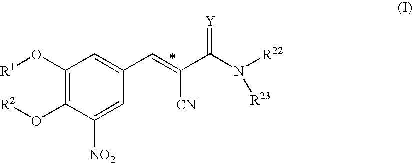

or in a preferred embodiment:

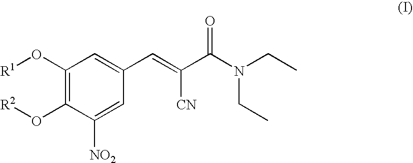

wherein the residues R¹, R², R²², R²³ and Y are as defined above, with the proviso that in the case that Y is oxygen, R²² is ethyl, R²³ is ethyl, and R² is H, then R³ in the residue R¹ is not tert-butyl. Preferred compounds of formula I are the preferred compounds of formula I mentioned above. One skilled in the art would understand that additional/alternative subsets of compounds of formula I can be derived by combining two or more of the above embodiments.

More examples of compounds are compounds 1 to 146, mentioned in table 1.

Further preferred compounds of formula I are compounds of formula I as defined before, wherein

each R³ is independently (C₁-C₁₀)-alkyl, (CR⁴R⁵)ₓ—R⁶, (C₁-C₈)-alkylene-(C₁-C₄)-alkoxy, (C₃-C₂₀)-alkenyl, (C₃-C₈)-alkynyl, (C₀-C₈)-alkylene-(C₃-C₁₄)-cycloalkyl, (C₀-C₈)-alkylene-(3-14-membered)-heterocycloalkyl, (C₀-C₈)-alkylene-(3-14-membered)-heterocycloalkenyl, (C₁-C₈)-alkylene-(C₃-C₁₄)-cycloalkenyl, (C₀-C₈)-alkylene-(C₆-C₁₄)-aryl, or (C₀-C₈)-alkylene-(5-14-membered)-heteroaryl, wherein the total carbon number of R³ is at most 15,
more preferably each R³ is independently (C₁-C₁₀)-alkyl, (CR⁴R⁵)ₓ—R⁶, (C₃-C₂₀)-alkenyl, (C₃-C₈)-alkynyl, (C₀-C₈)-alkylene-(C₃-C₁₄)-cycloalkyl, (C₀-C₈)-alkylene-(3-14-membered)-heterocycloalkyl, (C₀-C₈)-alkylene-(C₆-C₁₄)-aryl, or (C₀-C₈)-alkylene-(5-14-membered)-heteroaryl, wherein the total carbon number of R³ is at most 15.

The residues R⁴, R⁵ and R⁶ are mentioned before,

In a further embodiment of the present invention each R³ is independently selected from the group consisting of

(C₁-C₄)-alkyl, preferably methyl, ethyl, n-propyl, isopropyl, n-butyl, isobutyl, sec-butyl; (C₅-C₇)-alkyl; preferably C₅-alkyl, more preferably 1-ethyl-propyl and (C₈-C₂₀)-alkyl, preferably (C₈-C₁₂-alkyl), more preferably (C₈-C₁₀)-alkyl, most preferably 2-ethylhexyl, n-octyl,

In a further embodiment, each R³ of a compound according to formula I is C₅-C₇-alkyl, preferably C₅-alkyl, more preferably 1-ethyl-propyl.

In another embodiment of the invention, R³ of a compound according to formula I is selected from the group consisting of ethyl, isopropyl, 1-ethyl-propyl and isobutyl, or R³ is 2-ethylhexyl.

An example of a preferred embodiment of the invention relates to compounds selected from the group consisting of:
Carbonic acid 5-((E)-2-cyano-2-diethylcarbamoyl-vinyl)-2-hydroxy-3-nitro-phenyl ester isobutyl ester,
Carbonic acid 5-((E)-2-cyano-2-diethylcarbamoyl-vinyl)-2-hydroxy-3-nitro-phenyl ester phenethyl ester,
Carbonic acid 5-((E)-2-cyano-2-diethylcarbamoyl-vinyl)-2-hydroxy-3-nitro-phenyl ester pentyl ester,
Carbonic acid 5-((E)-2-cyano-2-diethylcarbamoyl-vinyl)-2-hydroxy-3-nitro-phenyl ester 1-methyl-pentyl ester,
Carbonic acid 5-((E)-2-cyano-2-diethylcarbamoyl-vinyl)-2-hydroxy-3-nitro-phenyl ester 1-ethyl-propyl ester,
Carbonic acid 5-((E)-2-cyano-2-diethylcarbamoyl-vinyl)-2-hydroxy-3-nitro-phenyl ester 2-ethyl-hexyl ester,
Carbonic acid butyl ester 5-((E)-2-cyano-2-diethylcarbamoyl-vinyl)-2-hydroxy-3-nitro-phenyl ester,
2-[5-((E)-2-Cyano-2-diethylcarbamoyl-vinyl)-2-hydroxy-3-nitro-phenoxycarbonyloxy]-2-methyl-propionic acid methyl ester,
Carbonic acid 5-((E)-2-cyano-2-diethylcarbamoyl-vinyl)-2-hydroxy-3-nitro-phenyl ester 8-diethylamino-octyl ester,
Carbonic acid 4-((E)-2-cyano-2-diethylcarbamoyl-vinyl)-2-nitro-6-p-tolyloxycarbonyloxy-phenyl ester p-tolyl ester,
Carbonic acid 4-((E)-2-cyano-2-diethylcarbamoyl-vinyl)-2-nitro-6-phenoxycarbonyloxy-phenyl ester phenyl ester,
Carbonic acid 5-((E)-2-cyano-2-diethylcarbamoyl-vinyl)-2-hydroxy-3-nitro-phenyl ester octyl ester,
Carbonic acid 4-((E)-2-cyano-2-diethylcarbamoyl-vinyl)-2-nitro-6-octyloxycarbonyloxy-phenyl ester octyl ester,
Carbonic acid 5-((E)-2-cyano-2-diethylcarbamoyl-vinyl)-2-hydroxy-3-nitro-phenyl ester propyl ester,
Carbonic acid 4-((E)-2-cyano-2-diethylcarbamoyl-vinyl)-2-nitro-6-propoxycarbonyloxy-phenyl ester propyl ester,
Carbonic acid 4-((E)-2-cyano-2-diethylcarbamoyl-vinyl)-2-(2-methoxy-phenoxycarbonyloxy)-6-nitro-phenyl ester 2-methoxy-phenyl ester,
Carbonic acid 4-((E)-2-cyano-2-diethylcarbamoyl-vinyl)-2-(4-methoxy-phenoxycarbonyloxy)-6-nitro-phenyl ester 4-methoxy-phenyl ester,
3-[5-((E)-2-Cyano-2-diethylcarbamoyl-vinyl)-2-hydroxy-3-nitro-phenoxycarbonyloxy]-2-methylene-butyric acid methyl ester,
Carbonic acid sec-butyl ester 5-((E)-2-cyano-2-diethylcarbamoyl-vinyl)-2-hydroxy-3-nitro-phenyl ester,
3-[5-((E)-2-Cyano-2-diethylcarbamoyl-vinyl)-2-hydroxy-3-nitro-phenoxycarbonyloxy]-pentanedioic acid diethyl ester,
Carbonic acid 5-((E)-2-cyano-2-diethylcarbamoyl-vinyl)-2-hydroxy-3-nitro-phenyl ester cyclohexylmethyl ester,
Carbonic acid 5-((E)-2-cyano-2-diethylcarbamoyl-vinyl)-2-hydroxy-3-nitro-phenyl ester (E)-octadec-9-enyl ester,
Carbonic acid 5-((E)-2-cyano-2-diethylcarbamoyl-vinyl)-2-hydroxy-3-nitro-phenyl ester 2-thiophen-2-yl-ethyl ester,
Carbonic acid 5-((E)-2-cyano-2-diethylcarbamoyl-vinyl)-2-hydroxy-3-nitro-phenyl ester 1-methyl-butyl ester,
Carbonic acid allyl ester 5-((E)-2-cyano-2-diethylcarbamoyl-vinyl)-2-hydroxy-3-nitro-phenyl ester,
Carbonic acid 5-((E)-2-cyano-2-diethylcarbamoyl-vinyl)-2-hydroxy-3-nitro-phenyl ester 1,7,7-trimethyl-bicyclo[2.2.1]hept-2-yl ester,
Carbonic acid 5-((E)-2-cyano-2-diethylcarbamoyl-vinyl)-2-hydroxy-3-nitro-phenyl ester (S)-3,7-dimethyl-oct-6-enyl ester,
Carbonic acid 5-((E)-2-cyano-2-diethylcarbamoyl-vinyl)-2-hydroxy-3-nitro-phenyl ester ethyl ester,
2-[5-((E)-2-Cyano-2-diethylcarbamoyl-vinyl)-2-hydroxy-3-nitro-phenoxycarbonyloxy]-butyric acid methyl ester,
Carbonic acid 5-((E)-2-cyano-2-diethylcarbamoyl-vinyl)-2-hydroxy-3-nitro-phenyl ester 2-isopropoxy-ethyl ester,
Carbonic acid 2-tert-butoxy-ethyl ester 5-((E)-2-cyano-2-diethylcarbamoyl-vinyl)-2-hydroxy-3-nitro-phenyl ester,
Carbonic acid benzyl ester 2-benzyloxycarbonyloxy-4-((E)-2-cyano-2-diethylcarbamoyl-vinyl)-6-nitro-phenyl ester,
Carbonic acid 5-((Z)-2-cyano-2-diethylcarbamoyl-vinyl)-2-hydroxy-3-nitro-phenyl ester 2-ethyl-hexyl ester,
Carbonic acid 5-((E)-2-cyano-2-diethylcarbamoyl-vinyl)-2-hydroxy-3-nitro-phenyl ester tetrahydro-furan-2-ylmethyl ester,
Carbonic acid 5-((E)-2-cyano-2-diethylcarbamoyl-vinyl)-2-hydroxy-3-nitro-phenyl ester 4-methyl-cyclohexyl ester,
Carbonic acid 5-((E)-2-cyano-2-diethylcarbamoyl-vinyl)-2-hydroxy-3-nitro-phenyl ester 2-methyl-cyclohexyl ester,
Carbonic acid 5-((E)-2-cyano-2-diethylcarbamoyl-vinyl)-2-hydroxy-3-nitro-phenyl ester 2,5-dimethyl-cyclohexyl ester,
Carbonic acid bicyclo[2.2.1]hept-2-yl ester 5-((E)-2-cyano-2-diethylcarbamoyl-vinyl)-2-hydroxy-3-nitro-phenyl ester,
Carbonic acid 5-((E)-2-cyano-2-diethylcarbamoyl-vinyl)-2-hydroxy-3-nitro-phenyl ester 2-(2-methoxy-phenyl)-ethyl ester,
Carbonic acid 5-((E)-2-cyano-2-diethylcarbamoyl-vinyl)-2-hydroxy-3-nitro-phenyl ester 2-(3-methoxy-phenyl)-ethyl ester,
3-[5-((E)-2-Cyano-2-diethylcarbamoyl-vinyl)-2-hydroxy-3-nitro-phenoxycarbonyloxy]-butyric acid tert-butyl ester,
Carbonic acid 5-((E)-2-cyano-2-diethylcarbamoyl-vinyl)-2-hydroxy-3-nitro-phenyl ester 2-(4-methoxy-phenyl)-1-methyl-ethyl ester,
Carbonic acid 5-((E)-2-cyano-2-diethylcarbamoyl-vinyl)-2-hydroxy-3-nitro-phenyl ester 1-methyl-2-phenyl-ethyl ester,
Carbonic acid 5-((E)-2-cyano-2-diethylcarbamoyl-vinyl)-2-hydroxy-3-nitro-phenyl ester 3,5-dimethyl-cyclohexyl ester,
Carbonic acid 5-((E)-2-cyano-2-diethylcarbamoyl-vinyl)-2-hydroxy-3-nitro-phenyl ester 2-(2-propoxy-ethoxy)-ethyl ester,
3-[5-((E)-2-Cyano-2-diethylcarbamoyl-vinyl)-2-hydroxy-3-nitro-phenoxycarbonyloxy]-butyric acid ethyl ester,
2-[5-((E)-2-Cyano-2-diethylcarbamoyl-vinyl)-2-hydroxy-3-nitro-phenoxycarbonyloxy]-cyclopentanecarboxylic acid methyl ester.

These are examples of an embodiment comprising compounds that have stability to chemical hydrolysis characterized a half-life t½ of over 3 hours at both pH 1.0 (0.1 N HCl) and pH 7.4 (PBS buffer). In contrast, half-life of a compound according to formula I wherein R² is H, R³ in the residue R¹ is tert-butyl, Y is oxygen, and R²² and R²³ are both ethyl is less than 1.0 h at pH 1.0 and 1.1 h at pH 7.4. Measurement of the half-life is further described below.

Suitable processes for the preparation of the compounds of formula I of the present invention are also already mentioned before.

The compounds of the present invention are suitable entacapone prodrugs. By providing the entacapone carbonates of formula I and pharmaceutical compositions comprising the compounds of formula I of the present invention it is possible to provide reproducible and constant plasma levels and/or to increase the bioavailability of entacapone.

It has been found by the inventors that by introducing a carbonate group in the m-position of the NO₂-group of entacapone the glucuronidation of entacapone and the following elimination of the glucuronide may be delayed.

The compounds of formula I of the present invention as well as the pharmaceutical compositions of the present invention are therefore useful compounds or compositions for the treatment and/or prophylaxis, preferably the treatment, of diseases associated with a disordered dopamine metabolism or an altered enzyme activity of COMT, preferably for the treatment and/or prophylaxis, preferably the treatment, of Parkinson's disease, restless leg syndrome, depression, or schizophrenia, more preferably for the treatment and/or prophylaxis, preferably the treatment, of Parkinson's disease or restless leg syndrome.

The compounds of formula I as mentioned in the present invention or the compositions as mentioned in the present invention are therefore especially useful for the treatment and/or prophylaxis, preferably the treatment, of the following diseases:
parkinson's disease
psychosis (e.g. schizophrenia)
mood disorders, such as depression, anxiety disorders (e.g. obsessional compulsive disorders, generalized anxiety) and aggressive disorders (including mixed aggressive-anxiety/depressive disorders)
restless leg syndrome
dopa-sensitive dyskinesia
apraxia induced by dopa or neuroleptica
neurodegenerative disorders
cognitive disorders
attention deficit hyperactivity disorder (ADHD)

In a preferred embodiment of the present invention the compounds of formula I or the compositions of the present invention are useful for the treatment and/or prophylaxis, preferably the treatment, of parkinson's disease, restless leg syndrome, psychosis (e.g. schizophrenia) and mood disorders such as depression, anxiety disorders (e.g. obsessional compulsive disorders, generalized anxiety) and aggressive disorders (including mixed aggressive-anxiety/depressive disorders), more preferably, the compounds of formula I or the compositions of the present invention are useful for the treatment and/or prophylaxis, preferably the treatment, of parkinson's disease or restless leg syndrome.

A further aspect of the present invention therefore relates to the use of the compounds of formula I or salts thereof as mentioned in the present application or the compositions of the present invention in the preparation of a medicament.

Further, the present invention relates to the use of the compounds of formula I or salts thereof as mentioned in the present application or the compositions of the present invention in the preparation of a medicament for inhibiting catechol-O-methyltransferase. The present invention further relates to a method of inhibiting catechol-O-methyltransferase comprising contacting catechol-O-methyltransferase with one or more compounds of formula I or salts thereof as mentioned in the present application or the compositions of the present invention.

In a further aspect, the present invention relates to the use of the compounds of formula I or salts thereof as mentioned in the present application or the compositions of the present invention in the preparation of a medicament for the treatment and/or prophylaxis, preferably the treatment, of diseases associated with a disordered dopamine metabolism.

In a preferred embodiment, the present invention relates to the use of compounds of formula I or salts thereof or the compositions of the present invention for the preparation of a medicament for the treatment and/or prophylaxis, preferably the treatment, of parkinson's disease, psychosis (e.g. schizophrenia), mood disorders such as depression, anxiety disorders (e.g. obsessional compulsive disorders, generalized anxiety) and aggressive disorders (including mixed aggressive-anxiety/depressive disorders), restless leg syndrome, dopa-sensitive dyskinesia, apraxia induced by dopa or neuroleptica, neurodegenerative disorders, cognitive disorders, attention deficite hyperactivity disorder (ADHD).

This aspect of the present invention may alternatively be formulated as a method for treatment and/or prophylaxis, preferably the treatment, of the diseases mentioned above in a human comprising administering to a human in need thereof an effective amount of a pharmaceutical product as described herein, which means a compound of formula I or a salt thereof or the compositions as disclosed in the present application.

In a further preferred embodiment, the present invention relates to the use or alternatively the method mentioned before, wherein the medicament additionally comprises beside the compound of formula I or a salt thereof as mentioned in the present application L-dopa and optionally a decarboxylase inhibitor such as carbidopa or benseracid, whereby the medicament preferably comprises L-dopa and the decarboxylase inhibitor mentioned before.

As already mentioned before, the treatment and/or prophylaxis, preferably the treatment, of the diseases mentioned above can in one embodiment be carried out by administering one dosage form of a medicament comprising the compound of formula I or a salt thereof as disclosed in the present application, L-dopa and optionally a decarboxylase inhibitor as mentioned before, whereby the presence of the decarboxylase inhibitor is preferred, or in a second embodiment, the administering of two dosage forms, one dosage form (medicament) comprising the compound of formula I or a salt thereof as disclosed in the present application and the other one comprising L-dopa and optionally a decarboxylase inhibitor such as carbidopa or benseracid. Therefore, L-dopa and a carbonate compound of the invention can be administered at the same or at separate times.

The present invention is illustrated by the following non-limiting examples.

### EXAMPLES

### Preparation

The compounds 1 to 146 as disclosed in table 1 are prepared by one of the following general methods:

### Method A

The compounds disclosed in table 1, wherein R² is H are prepared by method A.

Method A comprises the formation of a cyclic ester by means of phosgene, followed by selective ring opening via alcoholysis as shown in the specification before.

Entacapone (0.61 g, 2 mmol) is dissolved in dry toluene (20 mL). Pyridine (0.2 mL, 2 mmol) is added at ambient temperature. The reaction mixture is cooled in an ice bath and a 20% toluene solution of phosgene (4.55 g, 5.3 mL, 20 mmol) is added. A further 10 mL toluene was added. The mixture is agitated with an ice cooling for 1.5 h and 2 h at ambient temperature. The precipitate is removed by filtration and the filter cake is washed with a small amount of toluene. The filtrate is evaporated under vacuum. The resulting pale yellow solid is taken up in 20 mL of dichloromethane, and an excess of the appropriate alcohol is added. The reaction mixture is agitated at ambient temperature over night. Inprocess controls are done either by TLC or HCLC. After completion of the reaction, the yellow solution is washed twice with 50 mL portions of 2 N HCl and water, the organic layer dried over sodium sulphate and evaporated under vacuum to gain the crude compound either as a solid or as an oil which in most cases solidifies on standing. Typical yields of crude products are 60 to 80%. Purification is done by recrystallisation with hexane/ethyl acetate or by flash chromatography. The purified compounds are obtained as yellow to orange solids in 10 to 20% yields with purities ≧95%. The purity and identity is determined by HPLC/ESI-MS.

### Method B

The compounds disclosed in table 1, wherein R¹ and R² are a residue of formula II are prepared by method B.

Method B comprises the conversion of entacapone with chloroformic acid esters as shown in the specification before.

Entacapone (3.05 g, 10 mmol) and sodium carbonate (0.583 g, 7 mmol) is dissolved in water (17.4 mL). A nitrogen atmosphere is applied and the appropriate chloroformic ester (20% molar excess) is added dropwise with a syringe at ambient temperature. The mixture is agitated at ambient temperature until TLC control indicated the reaction went to completion (2 to 3 h). The reaction mixture is extracted twice with 200 mL portions of ethyl acetate, dried over sodium sulphate and the solvent is removed under vacuum to gain the crude product as an oil which solidifies on standing. Typical yields of the crude products are 70 to 80%. Purification is done by using a Flashmaster™ (column chromatography), to afford the purified compounds as yellow to orange solids in typical yields of 10 to 20%. The purity and identity is determined by HPLC/ISI-MS.

Entacapone which is used as starting material and reference compound is obtained by the following process:

### (i) Preparation of N1,N1-diethyl-2-cyanoacetamide

The reactor vessel is charged with 30 L dry THF under a nitrogen atmosphere, followed by the addition of diethylamine (1.272 kg, 17.39 mol). A 33% solution of n-hexyllithium in hexanes is transferred to a dropping funnel. The reactor vessel is cooled down to a temperature below −30° C. The n-hexyllithium solution is added drop wise to the reactor vessel. During the addition the temperature of the reaction mixture is kept well below −30° C. After completion of the addition the mixture is agitated at −30° C. for 1.5 h. In the meantime a second dropping funnel is charged with ethylcyanoacetate (0.655 kg, 5.79 mol), dissolved in THF (2 L). The ethylcyanoacetate solution is added drop wise to the reaction mixture at a temperature well below −30° C. After completion of the addition, the mixture is warmed to ambient temperature and agitated further 30 minutes at ambient temperature.

The reaction mixture is quenched by the drop wise addition of ethanol (0.66 L) and agitated overnight at ambient temperature. The solvents are removed by distillation under reduced pressure and the residue is treated with 10% HCl (8 L) and extracted twice with DCM (2×7 L). The combined DCM layers are washed with water (2×5 L). The DCM solution is aceotroped and evaporated to dryness, to gain the product as a brown oil (755.29 g, 97%) used in the next step without further purification

¹H-NMR, 200 MHz, CDCl₃: δ t 6H 1.2 ppm J 7 Hz, q 4H 3.4 ppm J 7 Hz, s 2H 3.55 ppm

### (ii) Preparation of N1,N1-diethyl-(E)-2-cyano-3-(4-hydroxy-3-methoxy-5-nitrophenyl)-2-propenamide

The total quantity of N1,N1-diethyl-2-cyanoacetamide isolated in step (i), dissolved in dry ethanol (5 L) is charged to a reactor vessel under a nitrogen atmosphere. A further amount of dry ethanol (25 L) is added to the reactor. After the addition of 5-nitrovanillin (965.6 g, 4.898 mol) and ammonium acetate (830.6 g, 10.775 mol), the suspension is agitated and heated to reflux until an in process control indicated the disappearance of 5-nitrovanillin. The dark solution is allowed to cool to ambient temperature, whereas a yellow solid precipitates. The mixture is cooled to −5° C. and agitated for 1 hour at −5° C. and the solid isolated by collection on a 20 L Buchner funnel under reduced pressure. The filter cake is washed with −10° C. ethanol (2 L). The solid is transferred to trays and dried at 30° C. in a vacuum oven, to afford a yellow solid (1.093 kg, 70%).

¹H-NMR, 200 MHz, DMSO-d₆: δ t 6H 1 ppm J 7 Hz, q 4H 3.45 ppm J 7 Hz, s 3H 3.65 ppm, bs 1H 5.7-6.1 ppm, s 1H 6.95, s 1H 7.5 ppm, s 1H 8.02 ppm.

### (iii) Preparation of N1,N1-diethyl-(E)-2-cyano-3-(3,4-dihydroxy-5-nitrophenyl)-2-propenamide (entacapone)

A reactor vessel is charged with dry chloroform (30 L), N1,N1-diethyl-(E)-2-cyano-3-(4-hydroxy-3-methoxy-5-nitrophenyl)-2-propenamide (1.083 kg, 3.392 mol) and aluminium chloride (0.498 kg, 3.731 mol). The suspension is agitated and cooled down to 0-5° C. Dry pyridine (1.180 kg, 14.923 mol), dissolved in dry chloroform (3 L) is transferred to a dropping funnel and added carefully, drop wise to the suspension. After complete addition, the reaction mixture is agitated and heated to reflux until an in process control indicates the ether cleavage to be complete. Most of the chloroform is distilled out of the reaction mixture. Water (7 L) is added and the residual chloroform is removed out of the two phase mixture by aceotropic distillation. The distilled water is transferred back to the reactor together with ethylacetate (25 L). The mixture is cooled down to 0-5° C. and 25% HCl (20 L) is added. The two phase mixture is agitated at ambient temperature for 30 minutes. After phase separation the watery layer is extracted twice with ethylacetate (2×12 L). The combined organic layers are distilled down to a final volume of 5 L. The product is precipitated by the addition of hexanes (10 L). After agitating for 1 hour the resulting solid is collected on a 20 L Buchner funnel by vacuum filtration. The filtercake is washed with water, transferred to trays and dried in a vacuum oven at 30° C. under reduced pressure, to gain the pure product as a green solid (0.581 kg, 56.1%)

Mp.: 161.05° C. HPLC: 99.37% E-Entacapone+0.19% Z-Entacapone. API/MS: [M+H]⁺ 306.17, [M+H]⁺-H2O=288.11, [M+H]⁺-Et=278.17, [M+H]⁺-NEt₂=233.

The compound is stored at ambient temperature in glass bottles over more than two years without degradation, confirmed by reanalysis by HPLC/MS and NMR.

An overview of the compounds of the invention according to formula I that were prepared utilizing general methods A, B, or C is provided by Table 1.

TABLE 1

| No. | Compound |
|---|---|
| 1 | Carbonic acid 5-((E)-2-cyano-2-diethylcarbamoyl-vinyl)-2-hydroxy-3-nitro-phenyl |
| | ester isobutyl ester |
| | 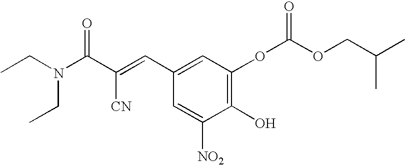 |
| | 1.02 (s, 3 H, CH₃); 1.03 (s, 3 H, CH₃); 1.28 (s (br), 6 H, N—CH₂—CH₃); 2.10 (m, |
| | 1 H, CH); 3.50 (s (br), 4 H, N—CH₂); 4.11 (d, J = 6.78 Hz, 2 H, O—CH₂); 7.59 (s, |
| | 1 H, CH═C); 8.16 (d, J = 2.25 Hz, 1 H, aromat.); 8.48 (d, J = 2.26 Hz, 1 H, |
| | aromat); 10.97 (s (br), 1 H, OH) |
| 2 | Carbonic acid 5-((E)-2-cyano-2-diethylcarbamoyl-vinyl)-2-hydroxy-3-nitro-phenyl |
| | ester phenethyl ester |
| | 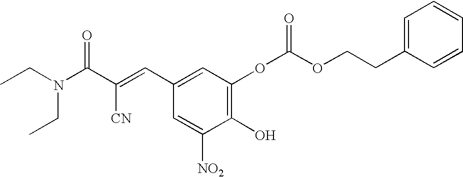 |
| | 1.27 (s (br), 6 H, N—CH₂—CH₃); 3.09 (t, J = 7.12 Hz, 2 H, O—CH₂—CH₂); 3.50 (s |
| | (br), 4 H, N—CH₂); 4.51 (t, J = 6.87 Hz, 2 H, O—CH₂); 7.25-7.28 (m, 3 H, |
| | aromat.); 7.32-7.36 (m, 2 H, aromat.); 7.58 (s, 1 H), CH═C); 8.12 (d, J = |
| | 2.29 Hz, 1 H, aromat.); 8.48 (d, J = 1.78 Hz, 1 H, aromat.); 10.92 (s (br), 0.9 H, |
| | OH) |
| 3 | Carbonic acid 4-((E)-2-cyano-2-diethylcarbamoyl-vinyl)-2-isobutoxycarbonyloxy- |
| | 6-nitro-phenyl ester isobutyl ester |
| | 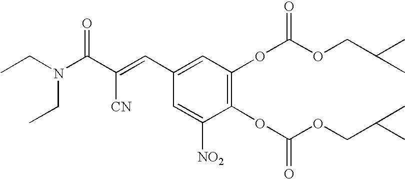 |
| 4 | Carbonic acid 2-chloro-benzyl ester 5-((E)-2-cyano-2-diethylcarbamoyl-vinyl)-2- |
| | hydroxy-3-nitro-phenyl ester |
| | 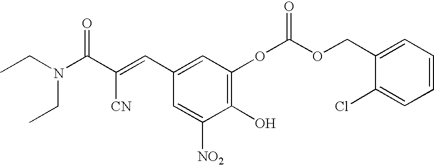 |
| | 1.27 (s (br), 6 H, N—CH₂—CH₃); 3.50 (s (br), 4 H, N—CH₂); 5.46 (s, 2 H, O—CH₂); |
| | 7.31-7.36 (m, 2 H, aromat.); 7.43-7.45 (m, 1 H, aromat.); 7.52-7.54 (m, 1 H, |
| | aromat.); 7.59 (s, 1 H, CH═C); 8.17 (d, J = 2.29 Hz, 1 H, aromat.); 8.50 (d, J = |
| | 2.29 Hz, 1 H, aromat.); 10.96 (s (br), 0.8 H, OH) |
| 5 | Carbonic acid tert-butyl ester 5-((E)-2-cyano-2-diethylcarbamoyl-vinyl)-2- |
| | hydroxy-3-nitro-phenyl ester |
| | 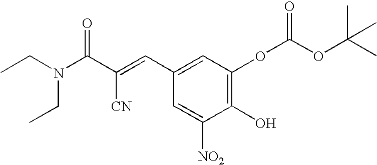 |
| | 1.27 (s (br), 6 H, N—CH₂—CH₃); 1.59 (s, 9 H, C(CH₃)₃); 3.50 (s (br), 4 H, N—CH₂); |
| | 7.58 (s, 1 H, CH═C); 8.13 (d, J = 2.26 Hz, 1 H, aromat.); 8.48 (d, J = 2.29 Hz, |
| | 1 H, aromat); 10.97 (s (br), 1 H, OH) |
| 6 | Carbonic acid 5-((E)-2-cyano-2-diethylcarbamoyl-vinyl)-2-hydroxy-3-nitro-phenyl |
| | ester pentyl ester |
| | 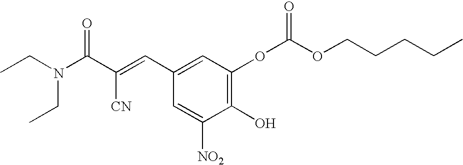 |
| | 0.95 (m, 7.38 Hz, 3 H, CH₃); 0.98 + 1.01 (d, J = 6.62 Hz, 1.5 H, CH₂); 1.28 (s |
| | (br), 6 H, N—CH₂—CH₃); 1.38-1.45 (m, 2 H, CH₂); 1.64-1.70 + 1.74-1.89 (m, |
| | 2.5 H, CH₂); 3.50 (s (br), 4 H, N—CH₂); 4.32 + 4.13 (t, J = 6.61 Hz, 1 H, O—CH₂); |
| | 4.35 + 4.21 (t, J = 6.86 Hz, 1 H, O—CH₂); 7.59 (s, 1 H, CH═C); 8.16 (d, J = 2.03 |
| | Hz, 1 H, aromat.); 8.48 (d, J = 2.04 Hz, 1 H, aromat.); 10.97 (s, 1 H, OH) |
| 7 | Carbonic acid 5-((E)-2-cyano-2-diethylcarbamoyl-vinyl)-2-hydroxy-3-nitro-phenyl |
| | ester isopropyl ester |
| | 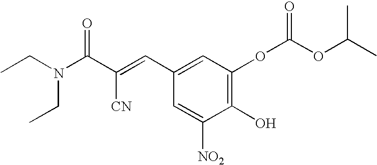 |
| | 1.28 (s (br), 6 H, N—CH₂—CH₃); 1.41 (s, 3 H, CH—CH₃); 1.43 (s 3 H, CH—CH₃); 3.50 |
| | (s (br), 4 H, N—CH₂); 5.02 (m, 1 H, O—CH); 7.59 (s, 1 H, CH═C); 8.16 (d, |
| | J = 2.04 Hz, 1 H, aromat.); 8.48 (d, J = 2.03 Hz, 1 H, aromat); 10.97 (s (br), 1 H, |
| | OH) |
| 8 | Carbonic acid 5-((E)-2-cyano-2-diethylcarbamoyl-vinyl)-2-hydroxy-3-nitro-phenyl |
| | ester 1-methyl-pentyl ester |
| | 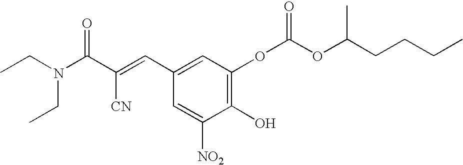 |
| | 0.93 (t, J = 7.12 Hz, 3 H, CH₃—CH₂); 1.27 (s (br), 6 H, N—CH₂—CH₃); 1.34-1.45 |
| | (m, 4 H, CH₂); 1.40 (d, J = 6.35 Hz, 3 H, CH—CH₃); 1.58-1.67 (m, 1 H, CH₂); |
| | 1.73-1.82 (m, 1 H, _{CH2}); 3.50 (s (br), 4 H, N—CH₂); 4.90 (m, 1 H, O—CH); 7.59 (s, |
| | 1 H, CH═C); 8.16 (d, J = 2.29 Hz, 1 H, aromat.); 8.48 (d, J = 2.04 Hz, 1 H, |
| | aromat.); 10.97 (s (br), 0.7 H, OH) |
| 9 | Carbonic acid 4-((E)-2-cyano-2-diethylcarbamoyl-vinyl)-2- |
| | isopropoxycarbonyloxy-6-nitro-phenyl ester isopropyl ester |
| | 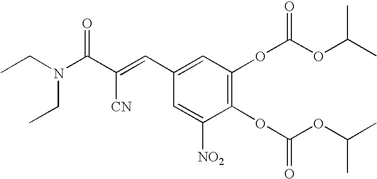 |
| 10 | Carbonic acid 5-((E)-2-cyano-2-diethylcarbamoyl-vinyl)-2-hydroxy-3-nito-phenyl |
| | ester 1-ethyl-propyl ester |
| | 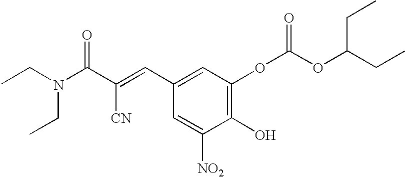 |
| | 1.01 (t, J = 7.53, 6 H, CH₃); 1.26 (s (br), 6 H, N—CH₂—CH₃); 1.74 (dt, J = |
| | 6.27/7.53 Hz, 4 H, CH—CH₂); 3.50 (s (br), 4 H, N—CH₂); 4.73 (m, J = 6.27 Hz, 1 H, |
| | O—CH); 7.59 (s, 1 H, CH═C); 8.15 (d, J = 2.26 Hz, 1 H, aromat.); 8.48 (d, J = |
| | 2.26 Hz, 1 H, aromat.); 10.96 (s, 0.9 H, OH) |
| 11 | Carbonic acid benzyl ester 5-((E)-2-cyano-2-diethylcarbamoyl-vinyl)-2-hydroxy-3- |
| | nitro-phenyl ester |
| | 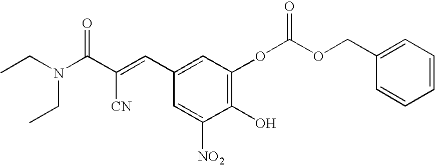 |
| | 1.27 (s (br), 6 H, N—CH₂—CH₃); 3.50 (s (br), 4 H, N—CH₂); 5.33 (s, 2 H, O—CH₂); |
| | 7.37-7.47 (m, 5 H, aromat.); 7.59 (s, 1 H, CH═C); 8.15 (d, J = 2.29 Hz, 1 H, |
| | aromat.); 8.49 (d, J = 2.29 Hz, 1 H, aromat.); 10.95 (s (br), 1 H, OH) |
| 12 | Carbonic acid 5-((E)-2-cyano-2-diethylcarbamoyl-vinyl)-2-hydroxy-3-nitro-phenyl |
| | ester 2,2-dimethyl-[1,3]dioxolan-4-ylmethyl ester |
| | 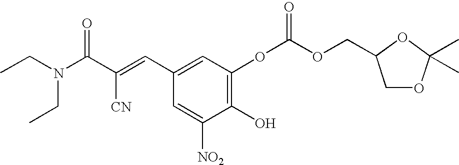 |
| 13 | Carbonic acid benzyl ester 2-benzyloxycarbonyloxy-4-((E)-2-cyano-2- |
| | diethylcarbamoyl-vinyl)-6-nitro-phenyl ester |
| | 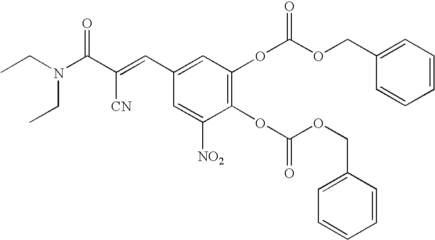 |
| 14 | [5-((E)-2-Cyano-2-diethylcarbamoyl-vinyl)-2-hydroxy-3-nitro- |
| | phenoxycarbonyloxy]-acetic acid ethyl ester |
| | 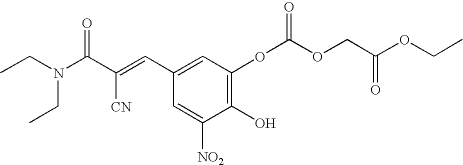 |
| | 1.28 (s (br), 6 H, N—CH₂—CH₃); 1.33 (t, J = 7.37 Hz, 3 H, CH₂—CH₃); 3.50 (s (br), |
| | 4 H, N—CH₂); 4.30 (q, J = 7.12 Hz, 2 H, O—CH₂); 4.78 (s, 2 H, CH₂—C═O); 7.59 (s, |
| | 1 H, CH═C); 8.15 (d, J = 2.29 Hz, 1 H, aromat.); 8.54 (d, J = 2.29 Hz, 1 H, |
| | aromat.); 10.99 (s, 0.9 H, OH) |
| 15 | Carbonic acid 5-((E)-2-cyano-2-diethylcarbamoyl-vinyl)-2-hydroxy-3-nitro-phenyl |
| | ester 2-ethyl-hexyl ester |
| | 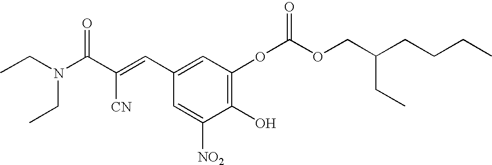 |
| | 0.92 (t, J = 7.03 Hz, 3 H, CH₃); 0.95 (t, J = 7.28 Hz, 3 H, CH₃); 1.29 (s (br), 6 H, |
| | N—CH₂—CH₃); 1.45-1.29 (m, 8 H, CH₂); 1.72 (m, J = 6.27 Hz, 1 H, O—CH); 3.50 |
| | (s (br), 4 H, N—CH₂); 4.24 (d, J = 6.03 Hz, 1 H, O—CH₂); 4.25 (d, J = 5.78 Hz, 1 H, |
| | O—CH₂); 7.59 (s, 1 H, CH═C); 8.15 (d, J = 2.26 Hz, 1 H, aromat.); 8.49 (d, J = |
| | 2.00 Hz, 1 H, aromat.); 10.97 (s, 1 H, OH) |
| 16 | 2-[5-((E)-2-Cyano-2-diethylcarbamoyl-vinyl)-2-hydroxy-3-nitro- |
| | phenoxycarbonyloxy]-propionic acid methyl ester |
| | 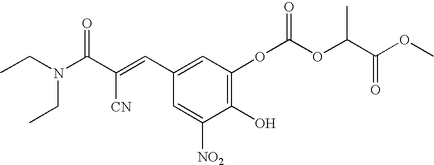 |
| | 1.28 (s (br), 6 H, N—CH₂—CH₃); 1.66 (d, J = 7.12 Hz, 3 H, CH—CH₃); 3.50 (s (br), |
| | 4 H, N—CH₂); 3.82 (s, 3 H, O—CH₃); 5.17 (q, J = 7.12 Hz, 1 H, O—CH); 7.59 (s, 1 H, |
| | CH═C); 8.16 (d, J = 2.29 Hz, 1 H, aromat.); 8.50 (d, J = 1.78 Hz, 1 H, aromat.), |
| | 10.97 (s, 0.9, OH) |
| 17 | Carbonic acid 4-((E)-2-cyano-2-diethylcarbamoyl-vinyl)-2-(2-ethyl- |
| | hexyloxycarbonyloxy)-6-nitro-phenyl ester 2-ethyl-hexyl ester |
| | 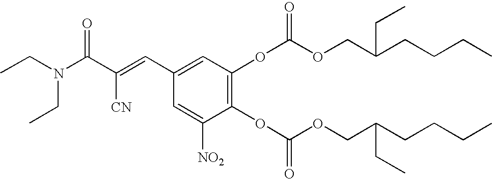 |
| 18 | Carbonic acid 5-((E)-2-cyano-2-diethylcarbamoyl-vinyl)-2-hydroxy-3-nitro-phenyl |
| | ester diethylcarbamoylmethyl ester |
| | 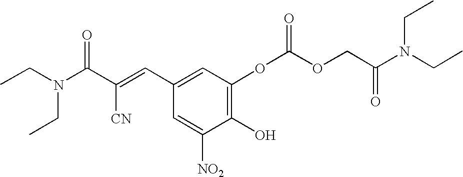 |
| | 1.16 (t, J = 7.12 Hz, 3 H, N—CH₂—CH₃); 1.24 (t, J = 7.13 Hz, 3 H, N—CH₂—CH₃); 1.28 |
| | (s (br), 6 H, N—CH₂—CH₃ [Entacapone]); 3.25 (q, J = 7.12 Hz, 2 H, N—CH₂); 3.44 (q, |
| | J = 7.12 Hz, 2 H, N—CH₂); 3.50 (s (br), 4 H, N—CH₂); 4.88 (s, 2 H, O—CH₂); 7.57 (s, |
| | 1 H, CH═C); 8.11 (d, J = 2.29 Hz, 1 H, aromat.); 8.57 (d, J = 2.03 Hz, 1 H, aromat.); |
| | 10.99 (s, 0.9 H, OH) |
| 19 | Carbonic acid butyl ester 5-((E)-2-cyano-2-diethylcarbamoyl-vinyl)-2-hydroxy-3- |
| | nitro-phenyl ester |
| | 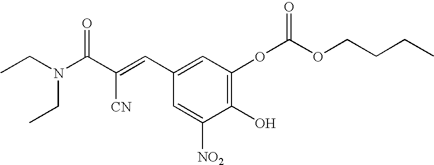 |
| | 0.99 (t, J = 7.38, 3 H, CH₃); 1.26 (s (br), 6 H, N—CH₂—CH₃); 1.48 (m, 2 H, CH₂); |
| | 1.77 (m, 2 H, CH₂); 3.50 (s (br), 4 H, N—CH₂); 4.32 (t, J = 6.86 Hz, 2 H, O—CH₂); |
| | 7.59 (s, 1 H, CH═C); 8.16 (d, J = 2.29 Hz, 1 H, aromat.); 8.48 (d, J = 2.29 Hz, |
| | 1 H, aromat.) |
| 20 | 2-[5-((E)-2-Cyano-2-diethylcarbamoyl-vinyl)-2-hydroxy-3-nitro- |
| | phenoxycarbonyloxy]-2-methyl-propionic acid methyl ester |
| | 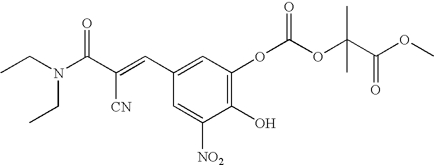 |
| | 1.29 (s (br), 6 H, N—CH₂—CH₃); 1.72 (s, 6 H, C—CH₃); 3.51 (s (br), 4 H, N—CH₂); |
| | 3.80 (s, 3 H, O—CH₃); 7.58 (s, 1 H, CH═C); 8.15 (s, 1 H, aromat.); 8.50 (s, 1 H, |
| | aromat.); 10.97 (s, 1 H, OH) |
| 21 | Carbonic acid 2-butoxycarbonyloxy-4-((E)-2-cyano-2-diethylcarbamoyl-vinyl)-6- |
| | nitro-phenyl ester butyl ester |
| | 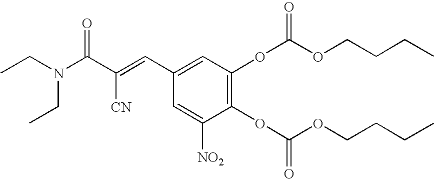 |
| 22 | 2-[5-((E)-2-Cyano-2-diethylcarbamoyl-vinyl)-2-hydroxy-3-nitro- |
| | phenoxycarbonyloxy]-propionic acid ethyl ester |
| | 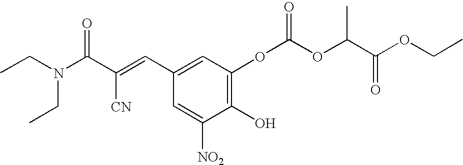 |
| | 1.27 (s (br), 6 H, N—CH₂—CH₃); 1.32 (t, J = 7.12 Hz, 3 H, O—CH₂—CH₃); 1.65 (d, J = |
| | 7.12 Hz, 3 H, CH—CH₃); 3.50 (s (br), 4 H, N—CH₂); 4.27 (q, J = 7.12 Hz, 2 H, |
| | O—CH₂); 5.13 (q, J = 7.12 Hz, 1 H, O—CH); 7.58 (s, 1 H, CH═C); 8.14 (d, |
| | J = 2.29 Hz, 1 H, aromat.); 8.53 (d, J = 2.29 Hz, 1 H, aromat.); 10.97 (s (br), |
| | 0.9 H, OH) |
| 23 | 2-[5-((E)-2-Cyano-2-diethylcarbamoyl-vinyl)-2-hydroxy-3-nitro- |
| | phenoxycarbonyloxy]-propionic acid ethyl ester |
| | 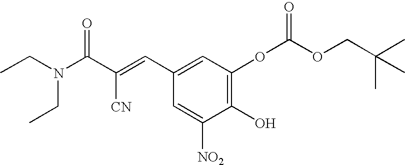 |
| 24 | 3-[5-((E)-2-Cyano-2-diethylcarbamoyl-vinyl)-2-hydroxy-3-nitro- |
| | phenoxycarbonyloxy]-2,2-dimethyl-propionic acid methyl ester |
| | 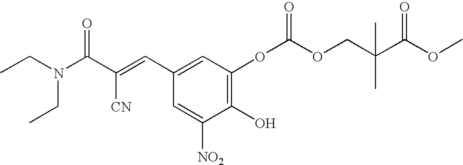 |
| | 1.27 (s (br), 6 H, N—CH₂—CH₃); 1.31 (s, 6 H, C(CH₃)₂); 3.50 (s (br), 4 H, N—CH₂); |
| | 3.75 (s, 3 H, O—CH₃); 4.36 (s, 2 H, O—CH₂); 7.59 (s, 1 H, CH═C); 8.14 (d, |
| | J = 2.29 Hz, 1 H, aromat.); 8.50 (d, J = 2.29 Hz, 1 H, aromat); 10.96 (s (br), 1 H, |
| | OH) |
| 25 | Carbonic acid 4-((E)-2-cyano-2-diethylcarbamoyl-vinyl)-2-(2,2-dimethyl- |
| | propoxycarbonyloxy)-6-nitro-phenyl ester 2,2-dimethyl-propyl ester |
| | 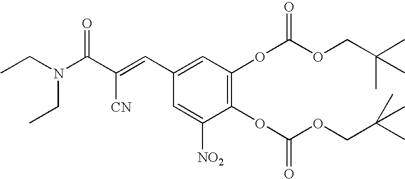 |
| 26 | 2-[5-((E)-2-Cyano-2-diethylcarbamoyl-vinyl)-2-hydroxy-3-nitro- |
| | phenoxycarbonyloxy]-2-methyl-propionic acid ethyl ester |
| | 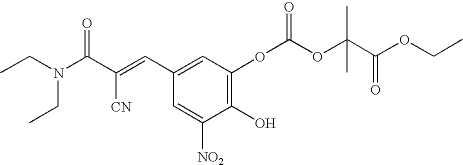 |
| 27 | Carbonic acid 5-((E)-2-cyano-2-diethylcarbamoyl-vinyl)-2-hydroxy-3-nitro-phenyl |
| | ester 4-nitro-benzyl ester |
| | 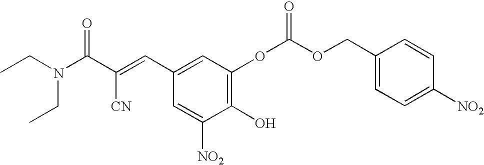 |
| 28 | Carbonic acid 5-((E)-2-cyano-2-diethylcarbamoyl-vinyl)-2-hydroxy-3-nitro- |
| | phenyl ester 2-propionylamino-ethyl ester |
| | 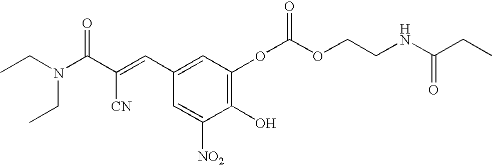 |
| 29 | Carbonic acid 4-((E)-2-cyano-2-diethylcarbamoyl-vinyl)-2-methoxycarbonyloxy- |
| | 6-nitro-phenyl ester methyl ester |
| | 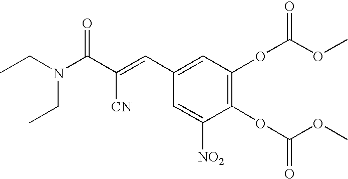 |
| 30 | Carbonic acid 5-((E)-2-cyano-2-diethylcarbamoyl-vinyl)-2-hydroxy-3-nitro-phenyl |
| | ester 8-diethylamino-octyl ester |
| | 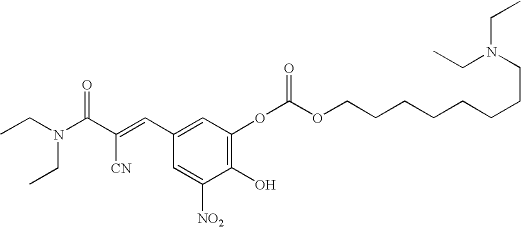 |
| 31 | Carbonic acid 4-((E)-2-cyano-2-diethylcarbamoyl-vinyl)-2-nitro-6-p- |
| | tolyloxycarbonyloxy-phenyl ester p-tolyl ester |
| | 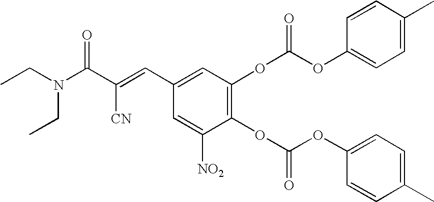 |
| 32 | Carbonic acid 5-((E)-2-cyano-2-diethylcarbamoyl-vinyl)-2-hydroxy-3-nitro-phenyl |
| | ester 2-acetylamino-ethyl ester |
| | 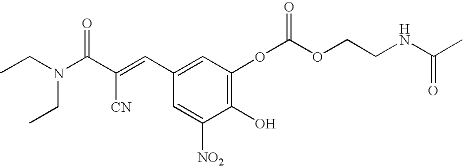 |
| 33 | Carbonic acid 4-((E)-2-cyano-2-diethylcarbamoyl-vinyl)-2-nitro-6- |
| | phenoxycarbonyloxy-phenyl ester phenyl ester |
| | 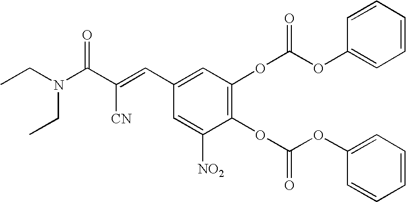 |
| 34 | 3-[5-((E)-2-Cyano-2-diethylcarbamoyl-vinyl)-2-hydroxy-3-nitro- |
| | phenoxycarbonyloxy]-butyric acid ethyl ester |
| | 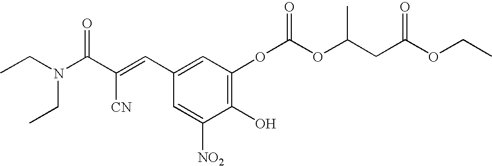 |
| 35 | Carbonic acid 5-((E)-2-cyano-2-diethylcarbamoyl-vinyl)-2-hydroxy-3-nitro-phenyl |
| | ester octyl ester |
| | 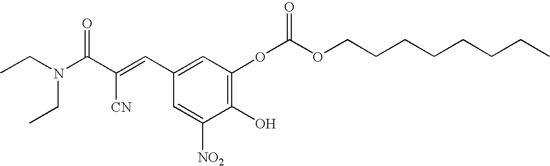 |
| 36 | Carbonic acid 5-((E)-2-cyano-2-diethylcarbamoyl-vinyl)-2-hydroxy-3-nitro-phenyl |
| | ester 4,4-dimethyl-2-oxo-tetrahydro-furan-3-yl ester |
| | 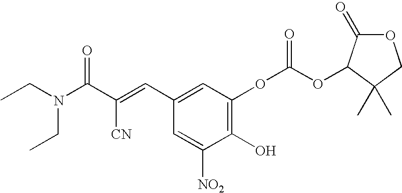 |
| | 1.25 (s, 3 H, C—CH₃); 1.27 (s (br), 6 H, N—CH₂—CH₃); 1.33 (s, 3 H, C—CH₃); 3.49 (s |
| | (br), 2 H, N—CH₂); 3.51 (s (br), 2 H, N—CH₂); 4.07 (q, ⁴J = 9.16 Hz, 1 H, O—CH₂); |
| | 4.12 (q, ⁴J = 9.16 Hz, 1 H, O—CH₂); 5.24 (s, 1 H, O—CH); 7.59 (s, 1 H, CH═C); |
| | 8.15 (d, J = 2.29 Hz, 1 H, aromat.); 8.56 (d, J = 2.29 Hz, 1 H, aromat.); 10.99 (s |
| | (br), 0.9 H, OH) |
| 37 | Carbonic acid 4-((E)-2-cyano-2-diethylcarbamoyl-vinyl)-2-nitro-6- |
| | octyloxycarbonyloxy-phenyl ester octyl ester |
| | 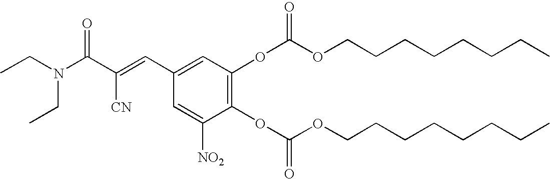 |
| 38 | Carbonic acid 5-((E)-2-cyano-2-diethylcarbamoyl-vinyl)-2-hydroxy-3-nitro- |
| | phenyl ester 5-oxo-tetrahydro-furan-3-yl ester |
| | 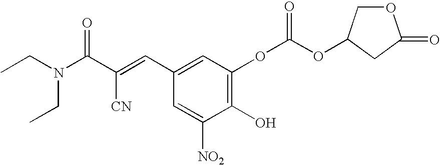 |
| 39 | Carbonic acid 4-((E)-2-cyano-2-diethylcarbamoyl-vinyl)-2-(naphthalen-1- |
| | yloxycarbonyloxy)-6-nitro-phenyl ester naphthalen-1-yl ester |
| | 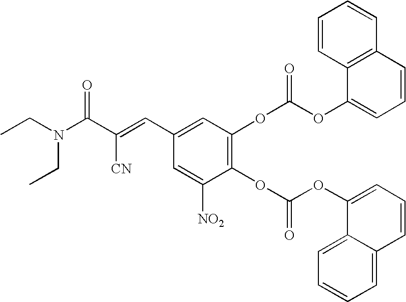 |
| 40 | Carbonic acid 5-((E)-2-cyano-2-diethylcarbamoyl-vinyl)-2-hydroxy-3-nitro-phenyl |
| | ester 5-oxo-tetrahydro-furan-3-yl ester |
| | 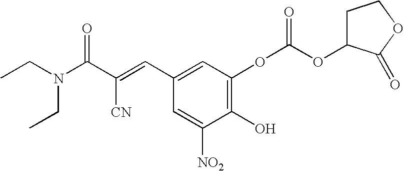 |
| 41 | Carbonic acid 5-((E)-2-cyano-2-diethylcarbamoyl-vinyl)-2-hydroxy-3-nitro-phenyl |
| | ester propyl ester |
| | 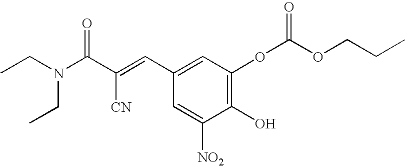 |
| | 1.04 (t, J = 7.38 Hz, 3 H, CH₃); 1.28 (s (br), 6 H, N—CH₂—CH₃); 1.81 (m, 2 H, |
| | O—CH₂—CH₂); 3.50 (s (br), 4 H, N—CH₂); 4.28 (t, J = 6.61 Hz, 2 H, O—CH₂); 7.59 (s, |
| | 1 H, CH═C); 8.16 (d, J = 2.29 Hz, 1 H, aromat); 8.48 (d, J = 2.29 Hz, 1 H, |
| | aromat.); 10.94 (s (br), 0.8 H, OH) |
| 42 | Carbonic acid 5-((E)-2-cyano-2-diethylcarbamoyl-vinyl)-2-hydroxy-3-nitro-phenyl |
| | ester 2,5-dimethyl-4-oxo-4,5-dihydro-furan-3-yl ester |
| | 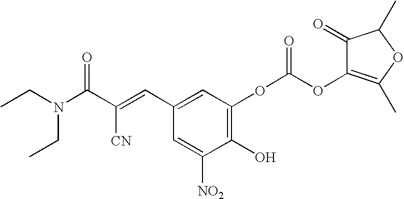 |
| 43 | Carbonic acid 4-((E)-2-cyano-2-diethylcarbamoyl-vinyl)-2-nitro-6- |
| | propoxycarbonyloxy-phenyl ester propyl ester |
| | 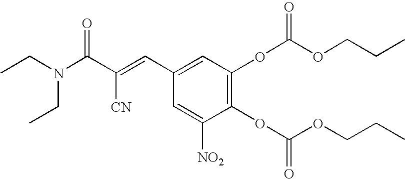 |
| 44 | 2-Acetylamino-3-[5-((E)-2-cyano-2-diethylcarbamoyl-vinyl)-2-hydroxy-3-nitro- |
| | phenoxycarbonyloxy]-propionic acid methyl ester |
| | 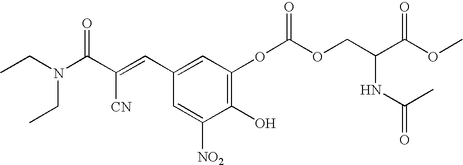 |
| 45 | Carbonic acid 4-((E)-2-cyano-2-diethylcarbamoyl-vinyl)-2-(2-methoxy- |
| | phenoxycarbonyloxy)-6-nitro-phenyl ester 2-methoxy-phenyl ester |
| | 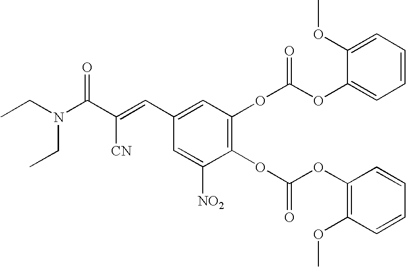 |
| 46 | [5-((E)-2-Cyano-2-diethylcarbamoyl-vinyl)-2-hydroxy-3-nitro- |
| | phenoxycarbonyloxy]-acetic acid methyl ester |
| | 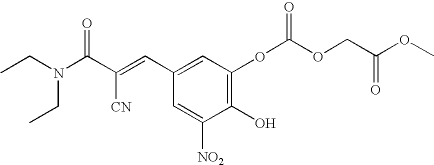 |
| | 1.26 (s (br), 6 H, N—CH₂—CH₃); 3.50 (s (br), 4 H, N—CH₂); 3.84 (s, 3 H, O—CH₃); |
| | 4.80 (s, 2 H, O—CH₂); 7.59 (s, 1 H, CH═C); 8.15 (d, J = 2.03 Hz, 1 H, aromat.); |
| | 8.53 (d, J = 1.78 Hz, 1 H, aromat.); 10.96 (s (br), 1 H, OH) |
| 47 | Carbonic acid 4-((E)-2-cyano-2-diethylcarbamoyl-vinyl)-2-(4-methoxy- |
| | phenoxycarbonyloxy)-6-nitro-phenyl ester 4-methoxy-phenyl ester |
| | 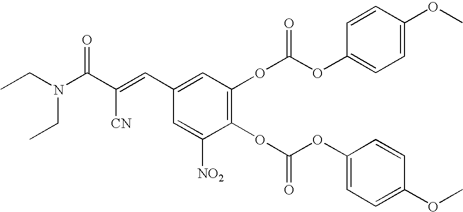 |
| 48 | 3-[5-((E)-2-Cyano-2-diethylcarbamoyl-vinyl)-2-hydroxy-3-nitro- |
| | phenoxycarbonyloxy]-2-methylene-butyric acid methyl ester |
| | 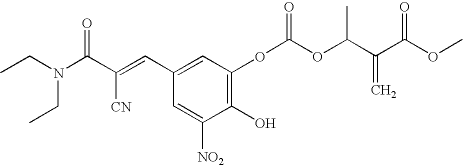 |
| | 1.27 (t, J = 7.12 Hz, 6 H, N—CH₂—CH₃); 1.57 (d, J = 6.36 Hz, 3 H, CH—CH₃); 3.50 |
| | (q, J = 7.12 Hz, 4 H, N—CH₂); 3.81 (s, 3 H, O—CH₃); 5.71 (q, J = 6.61 Hz, 1 H, |
| | O—CH); 6.00 (s, 1 H, C═CH₂); 6.40 (s, 1 H, C═CH₂); 7.56 (s, 1 H, CH═C); 8.12 (d, |
| | J = 2.29 Hz, 1 H, aromat.); 8.47 (d, J = 2.29 Hz, 1 H, aromat.); 10.90 (s, 0.8 H, |
| | OH) |
| 49 | Carbonic acid 4-((E)-2-cyano-2-diethylcarbamoyl-vinyl)-2- |
| | isopropenyloxycarbonyloxy-6-nitro-phenyl ester isopropenyl ester |
| | 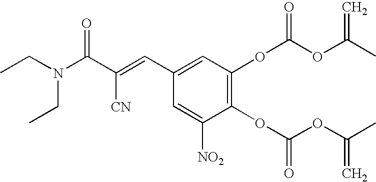 |
| 50 | [5-((E)-2-Cyano-2-diethylcarbamoyl-vinyl)-2-hydroxy-3-nitro- |
| | phenoxycarbonyloxy]-phenyl-acetic acid ethyl ester |
| | 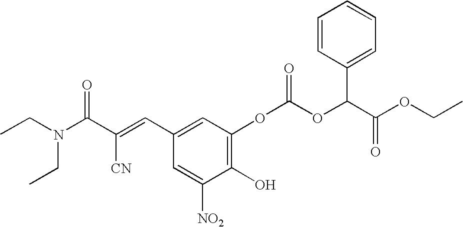 |
| | 1.25 (t, J = 7.12 Hz, 3 H, O—CH₂—CH₃); 1.26 (s (br), 6 H, N—CH₂—CH₃); 3.50 (s, |
| | (br), 4 H, N—CH₂); 4.18-4.33 (m, 2 H, O—CH₂); 5.95 (s, 1 H, O—CH); 7.41-7.45 |
| | (m, 3 H, aromat.); 7.52-7.54 (m, 2 H, aromat.); 7.58 (s, 1 H, CH═C); 8.13 (d, J = |
| | 2.54 Hz, 1 H, aromat.); 8.55 (d, J = 2.29 Hz, 1 H, aromat.); 10.94 (s (br), 0.8 H, |
| | OH) |
| 51 | Carbonic acid 5-((E)-2-cyano-2-diethylcarbamoyl-vinyl)-2-hydroxy-3-nitro-phenyl |
| | ester cyclohexyl ester |
| | 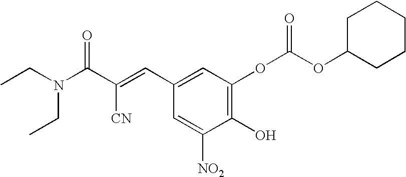 |
| | 1.28 (s (br), 6 H, N—CH₂—CH₃); 1.36-1.46 (m, 2 H, CH₂); 1.55-1.66 (m, 4 H, |
| | CH₂); 1.79-1.84 (m, 2 H, CH—CH₂); 2.00-2.04 (m, 2 H, CH—CH₂); 3.50 (s (br), |
| | 4 H, N—CH₂); 4.77 (m, J = 4.07 Hz, 1 H, O—CH); 7.69 (s, 1 H, CH═C); 8.16 (d, J = |
| | 2.29 Hz, 1 H, aromat.); 8.48 (d, J = 2.29 Hz, 1 H, aromat.); 10.97 (s, 1 H, OH) |
| 52 | [5-((E)-2-Cyano-2-diethylcarbamoyl-vinyl)-2-hydroxy-3-nitro- |
| | phenoxycarbonyloxy]-phenyl-acetic acid methyl ester |
| | 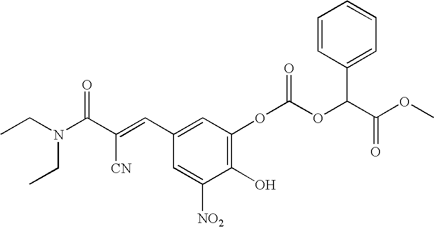 |
| | 1.27 (s (br), 6 H, N—CH₂—CH₃); 3.50 (s (br), 4 H, N—CH₂); 3.79 (s, 3 H, O—CH₃); |
| | 5.98 (s, 1 H, O—CH); 7.42-7.45 (m, 3 H, aromat.); 7.51-7.54 (m, 2 H, aromat.); |
| | 7.58 (s, 1 H, CH═C); 8.14 (d, J = 2.29 Hz, 1 H, aromat.); 8.54 (d, J = 2.29 Hz, |
| | 1 H, aromat.); 10.95 (s, 1 H, OH) |
| 53 | Carbonic acid sec-butyl ester 5-((E)-2-cyano-2-diethylcarbamoyl-vinyl)-2- |
| | hydroxy-3-nitro-phenyl ester |
| | 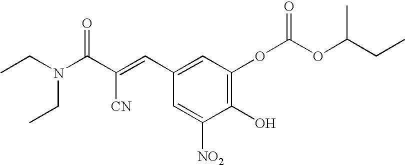 |
| 54 | [5-((E)-2-Cyano-2-diethylcarbamoyl-vinyl)-2-hydroxy-3-nitro- |
| | phenoxycarbonyloxy]-phenyl-acetic acid benzyl ester |
| | 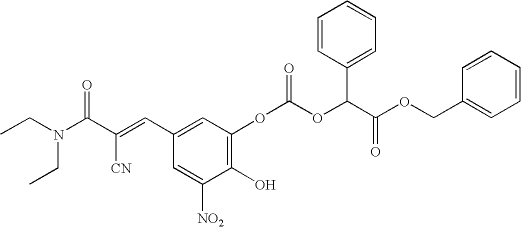 |
| | 1.27 (s (br), 6 H, N—CH₂—CH₃); 3.49 (s (br), 2 H, N—CH₂); 3.51 (s (br), 2 H, |
| | N—CH₂); 5.22 (s, 2 H, O—CH₂); 6.01 (s, 1 H, O—CH); 7.22-7.24 (m, 2 H, aromat.); |
| | 7.29-7.32 (m, 3 H, aromat.); 7.41-7.43 (m, 3 H, aromat.); 7.50-7.52 (m, 2 H, |
| | aromat.); 7.57 (s, 1 H, CH═C); 8.06 (d, J = 2.29 Hz, 1 H, aromat.); 8.55 (d, J = |
| | 2.29 Hz, 1 H, aromat.); 10.83 (s, 1 H, OH) |
| 55 | Carbonic acid 5-((E)-2-cyano-2-diethylcarbamoyl-vinyl)-2-hydroxy-3-nitro-phenyl |
| | ester 2-diethylamino-ethyl ester |
| | 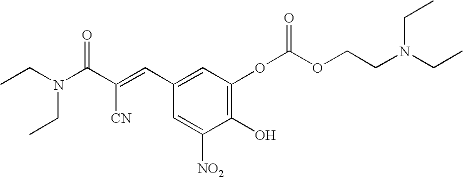 |
| 56 | 3-[5-((E)-Cyano-2-diethylcarbamoyl-vinyl)-2-hydroxy-3-nitro- |
| | phenoxycarbonyloxy]-pentanedioic acid diethyl ester |
| | 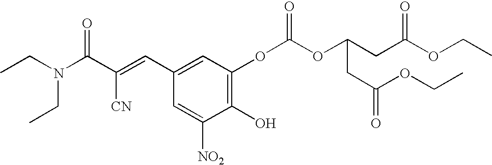 |
| | 1.26 (s (br), 6 H, N—CH₂—CH₃); 1.29 (t, J = 7.12 Hz, 6 H, O—CH₂—CH₃); 2.85 (d, |
| | J = 6.10 Hz, 2 H, CH—CH₂); 2.86 (d, J = 6.10 Hz, 2 H, CH—CH₂); 3.49 (s, 2 H, |
| | N—CH₂); 3.51 (s, 2 H, N—CH₂); 4.20 (q, J = 7.12 Hz, 4 H, O—CH₂); 5.54 (m, |
| | J = 6.36 Hz, 1 H, O—CH); 7.58 (s, 1 H, CH═C); 8.11 (d, J = 2.29 Hz, 1 H, |
| | aromat.); 8.52 (d, J = 2.29 Hz, 1 H, aromat.); 10.94 (s (br), 0.9 H, OH) |
| 57 | Carbonic acid 5-((E)-2-cyano-2-diethylcarbamoyl-vinyl)-2-hydroxy-3-nitro-phenyl |
| | ester cyclohexylmethyl ester |
| | 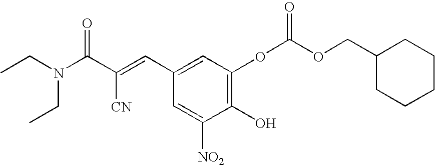 |
| 58 | 3-[5-((E)-2-Cyano-2-diethylcarbamoyl-vinyl)-2-hydroxy-3-nitro- |
| | phenoxycarbonyloxy]-pentanedioic acid diethyl ester |
| | 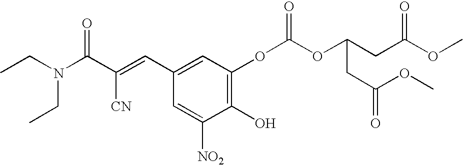 |
| | 1.26 (s (br), 6 H, N—CH₂—CH₃); 2.87 (d, J = 6.35 Hz, 2 H, CH—CH₂); 2.89 (d, J = |
| | 6.35 Hz, 2 H, CH—CH₂); 3.49 (s, 2 H, N—CH₂); 3.51 (s, 2 H, N—CH₂); 3.74 (s, 6 H, |
| | O—CH₃); 5.54 (m, J = 6.10 Hz, 1 H, O—CH); 7.58 (s, 1 H, CH═C); 8.13 (d, |
| | J = 2.29 Hz, 1 H, aromat.); 8.51 (d, J = 2.29 Hz, 1 H, aromat.); 10.95 (s (br), |
| | 0.9 H, OH) |
| 59 | Carbonic acid 5-((E)-2-cyano-2-diethylcarbamoyl-vinyl)-2-hydroxy-3-nitro-phenyl |
| | ester (E)-octadec-9-enyl ester |
| | 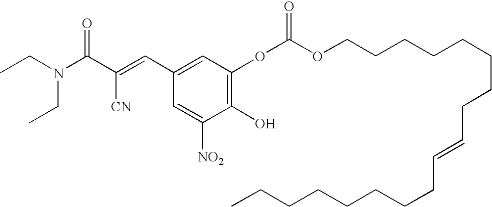 |
| 60 | 1-[5-((E)-Cyano-2-diethylcarbamoyl-vinyl)-2-hydroxy-3-nitro- |
| | phenoxycarbonyloxy]-cyclopropanecarboxylic acid ethyl ester |
| | 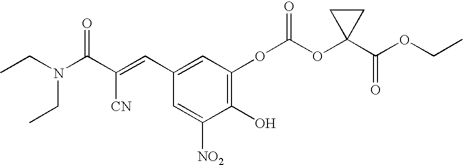 |
| | 1.27 (s (br), 6 H, N—CH₂—CH₃); 1.30 (t, J = 7.13 Hz, 3 H, O—CH₂—CH₃); 1.44 (m, J = |
| | 5.85/3.30 Hz, 2 H, cyclopropyl-CH₂); 1.62 (m, J = 5.43/3.31 Hz, 2 H, |
| | cyclopropyl-CH₂); 3.49 (s (br), 2 H, N—CH₂); 3.51 (s (br), 2 H, N—CH₂); 4.26 (q, |
| | J = 7.12 Hz, 2 H, O—CH₂); 7.58 (s, 1 H, CH═C); 8.15 (d, J = 2.29 Hz, 1 H, |
| | aromat.); 8.51 (d, J = 2.29 Hz, 1 H, aromat.); 10.97 (s, 1 H, OH) |
| 61 | Carbonic acid 5-((E)-2-cyano-2-diethylcarbamoyl-vinyl)-2-hydroxy-3-nitro-phenyl |
| | ester 2-thiophen-2-yl-ethyl ester |
| | 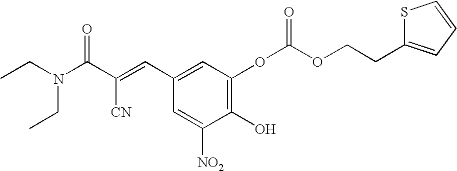 |
| 62 | 1-[5-((E)-2-Cyano-2-diethylcarbamoyl-vinyl)-2-hydroxy-3-nitro- |
| | phenoxycarbonyloxy]-cyclopropanecarboxylic acid methyl ester |
| | 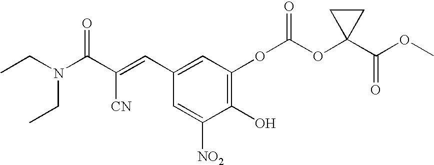 |
| | 1.27 (s (br), 6 H, N—CH₂—CH₃); 1.47 (m, J = 5.84/3.31 Hz, 2 H, cyclopropyl-CH₂); |
| | 1.64 (m, J = 5.34/3.31 Hz, 2 H, cyclopropyl-CH₂); 3.49 (s (br), 2 H, N—CH₂); 3.51 |
| | (s (br), 2 H, N—CH₂); 3.81 (s, 3 H, O—CH₃); 7.58 (s, 1 H, CH═C); 8.17 (d, J = 2.04 |
| | Hz, 1 H, aromat.); 8.51 (d, J = 2.29 Hz, 1 H, aromat.); 10.97 (s, 0.9 H, OH) |
| 63 | Carbonic acid 5-((E)-2-cyano-2-diethylcarbamoyl-vinyl)-2-hydroxy-3-nitro-phenyl |
| | ester 1-methyl-butyl ester |
| | 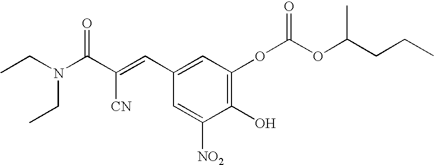 |
| | 0.97 (t, J = 6.94 Hz, 3 H, CH₃); 1.29 (s (br), 6 H, N—CH₂—CH₃); 1.39 (d, J = 6.30 |
| | Hz, 3 H, O—CH—CH₃); 1.41-1.52 (m, 2 H, CH₂); 1.56-1.63 (m, 1 H, CH₂); 1.73- |
| | 1.80 (m, 1 H, CH₂); 3.50 (s (br), 4 H, N—CH₂); 4.91 (m, 1 H, O—CH); 7.59 (s, 1 H, |
| | CH═C); 8.16 (d, J = 2.52 Hz, 1 H, aromat.); 8.48 (d, J = 1.89 Hz, 1 H, aromat.); |
| | 10.96 (s (br), 0.8 H, OH) |
| 64 | 2-[5-((E)-2-Cyano-2-diethylcarbamoyl-vinyl)-2-hydroxy-3-nitro- |
| | phenoxycarbonyloxy]-pentanoic acid ethyl ester |
| | 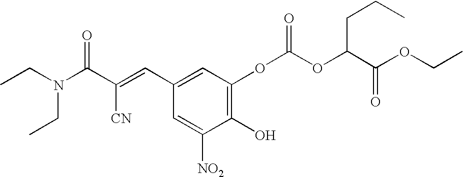 |
| | 1.00 (t, J = 7.38 Hz, 3 H, CH₃); 1.27 (s (br), 6 H, N—CH₂—CH₃); 1.31 (t, |
| | J = 7.12 Hz, 3 H, O—CH₂—CH₃); 1.50-1.60 (m, 2 H, CH₂); 1.93-1.98 (m, 2 H, |
| | CH₂); 3.49 (s (br), 2 H, N—CH₂); 3.51 (s (br), 2 H, N—CH₂); 4.27 (q, J = 7.12 Hz, |
| | 2 H, O—CH₂); 5.04 (t, J = 6.36 Hz, 1 H, O—CH); 7.58 (s, 1 H, CH═C); 8.14 (d, |
| | J = 2.29 Hz, 1 H, aromat.); 8.52 (d, J = 2.29 Hz, 1 H, aromat.); 10.96 (s (br), 1 H, |
| | OH) |
| 65 | Carbonic acid allyl ester 5-((E)-2-cyano-2-diethylcarbamoyl-vinyl)-2-hydroxy-3- |
| | nitro-phenyl ester |
| | 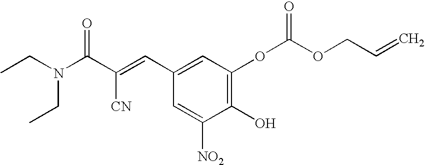 |
| | 1.28 (s (br), 6 H, N—CH₂—CH₃); 3.50 (s (br), 4 H, N—CH₂); 4.80 (d, J = 5.67 Hz, |
| | 2 H, O—CH₂); 5.38 (dd, J = 1.26/10.09 Hz, 1 H, CH₂═CH); 5.47 (dd, |
| | J = 1.26/17.02 Hz, 1 H, CH₂═CH); 5.97-6.05 (m, 1 H, CH₂═CH); 7.59 (s, 1 H, |
| | CH═C); 8.16 (d, J = 2.53 Hz, 1 H, aromat.); 8.49 (d, J = 1.89 Hz, 1 H, aromat.); |
| | 10.97 (s (br), 0.8 H, OH) |
| 66 | 3-[5-((E)-2-Cyano-2-diethylcarbamoyl-vinyl)-2-hydroxy-3-nitro- |
| | phenoxycarbonyloxy]-hexanoic acid methyl ester |
| | 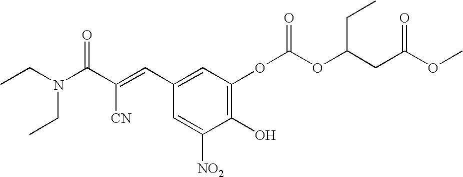 |
| | 1.04 (t, J = 7.37 Hz, 3 H, CH₃); 1.28 (s (br), 6 H, N—CH₂—CH₃); 1.81 (m, 2 H, |
| | CH—CH₂—CH₃); 2.72 (m, 2 H, CH₂—C═O); 3.49 (s (br), 2 H, N—CH₂); 3.51 (s (br), 2 H, |
| | N—CH₂); 3.73 (s, 3 H, O—CH₃); 5.18 (m, J = 7.89 Hz, 1 H, O—CH); 7.58 (s, 1 H, |
| | CH═C); 8.12 (d, J = 2.29 Hz, 1 H, aromat.); 8.50 (d, J = 2.29 Hz, 1 H, aromat.); |
| | 10.94 (s (br), 0.9 H, OH) |
| 67 | Carbonic acid 5-((E)-2-cyano-2-diethylcarbamoyl-vinyl)-2-hydroxy-3-nitro-phenyl |
| | ester 1,7,7-trimethyl-bicyclo[2.2.1]hept-2-yl ester |
| | 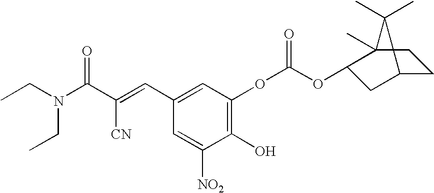 |
| 68 | 1-[5-((E)-2-Cyano-2-diethylcarbamoyl-vinyl)-2-hydroxy-3-nitro- |
| | phenoxycarbonyloxy]-cyclobutanecarboxylic acid methyl ester |
| | 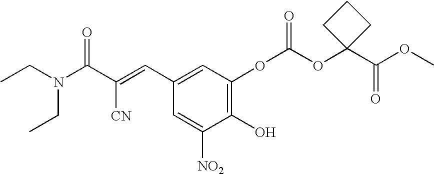 |
| 69 | Carbonic acid 5-((E)-2-cyano-2-diethylcarbamoyl-vinyl)-2-hydroxy-3-nitro-phenyl |
| | ester (S)-3,7-dimethyl-oct-6-enyl ester |
| | 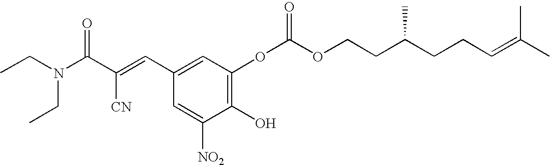 |
| 70 | 1-[5-((E)-2-Cyano-2-diethylcarbamoyl-vinyl)-2-hydroxy-3-nitro- |
| | phenoxycarbonyloxy]-cyclopentanecarboxylic acid methyl ester |
| | 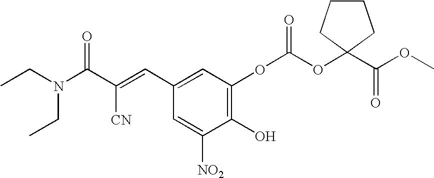 |
| 71 | Carbonic acid 5-((E)-2-cyano-2-diethylcarbamoyl-vinyl)-2-hydroxy-3-nitro-phenyl |
| | ester ethyl ester |
| | 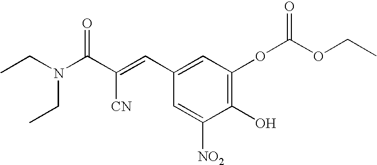 |
| | 1.27 (s (br), 6 H, N—CH₂—CH₃); 1.43 (t, J = 7.12 Hz, 3 H, O—CH₂—CH₃); 3.50 (s |
| | (br), 4 H, N—CH₂); 4.38 (q, J = 7.12 Hz, 2 H, O—CH₂); 7.69 (s, 1 H, CH═C); 8.17 |
| | (d, J = 2.29 Hz, 1 H, aromat.); 8.48 (d, J = 2.03 Hz, 1 H, aromat.); 10.97 (s (br), |
| | 1 H, OH) |
| 72 | 2-[5-((E)-2-Cyano-2-diethylcarbamoyl-vinyl)-2-hydroxy-3-nitro- |
| | phenoxycarbonyloxy]-butyric acid methyl ester |
| | 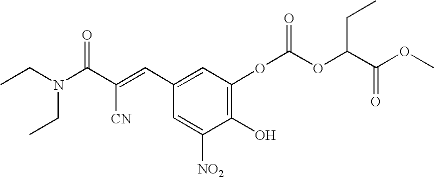 |
| 73 | Carbonic acid 5-((E)-2-cyano-2-diethylcarbamoyl-vinyl)-2-hydroxy-3-nitro-phenyl |
| | ester 2-isopropoxy-ethyl ester |
| | 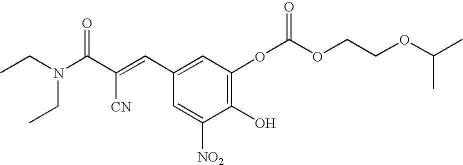 |
| 74 | 3-[5-((E)-2-Cyano-2-diethylcarbamoyl-vinyl)-2-hydroxy-3-nitro- |
| | phenoxycarbonyloxy]-butyric acid methyl ester |
| | 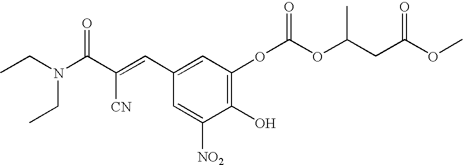 |
| 75 | Carbonic acid 2-tert-butoxy-ethyl ester 5-((E)-2-cyano-2-diethylcarbamoyl-vinyl)- |
| | 2-hydroxy-3-nitro-phenyl ester |
| | 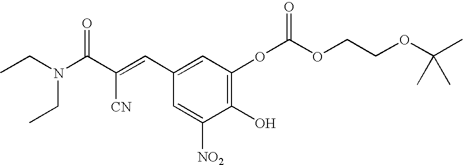 |
| 76 | Carbonic acid 5-((E)-2-cyano-2-diethylcarbamoyl-vinyl)-2-hydroxy-3-nitro-phenyl |
| | ester 3,3-dimethyl-butyl ester |
| | 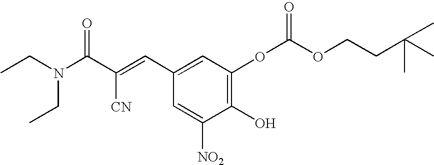 |
| 77 | Carbonic acid 5-((E)-2-cyano-2-diethylcarbamoyl-vinyl)-2-hydroxy-3-nitro-phenyl |
| | ester prop-2-ynyl ester |
| | 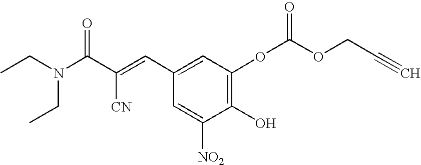 |
| | 1.27 (s (br), 6 H, N—CH₂—CH₃); 2.63 (t, J = 2.29 Hz, 1 H, CH≡C); 3.50 (s (br), 4 H, |
| | N—CH₂); 4.90 (s, 1 H, O—CH₂); 4.91 (s, 1 H, O—CH₂); 7.60 (s, 1 H, CH═C); 8.17 (d, |
| | J = 2.28 Hz, 1 H, aromat.); 8.50 (d, J = 2.29 Hz, 1 H, aromat.); 10.97 (s (br), |
| | 0.8 H, OH) |
| 78 | 2-[5-((E)-2-Cyano-2-diethylcarbamoyl-vinyl)-2-hydroxy-3-nitro- |
| | phenoxycarbonyloxy]-2-ethyl-butyric acid methyl ester |
| | 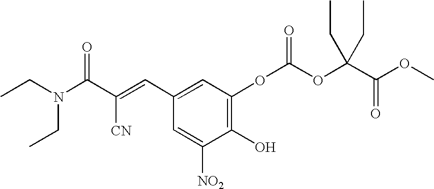 |
| 79 | Carbonic acid but-2-ynyl ester 5-((E)-2-cyano-2-diethylcarbamoyl-vinyl)-2- |
| | hydroxy-3-nitro-phenyl ester |
| | 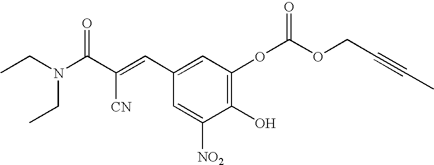 |
| | 1.27 (s (br), 6 H, N—CH₂—CH₃); 1.91 (m, 3 H, CH₃—C≡C); 3.50 (s (br), 4 H, |
| | N—CH₂); 4.87 (m, 2 H, O—CH₂); 7.59 (s, 1 H, CH≡C); 8.16 (d, J = 2.29 Hz, 1 H, |
| | aromat.); 8.50 (d, J = 2.28 Hz, 1 H, aromat.); 10.97 (s (br), 0.8 H, OH) |
| 80 | Carbonic acid 5-((Z)-2-cyano-2-diethylcarbamoyl-vinyl)-2-hydroxy-3-nitro-phenyl |
| | ester 2-ethyl-butyl ester |
| | 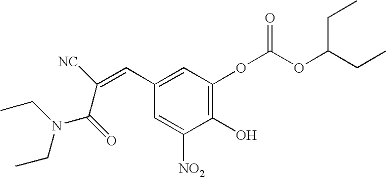 |
| 81 | Carbonic acid 5-((E)-2-cyano-2-diethylcarbamoyl-vinyl)-2-hydroxy-3-nitro-phenyl |
| | ester 4-methoxy-benzyl ester |
| | 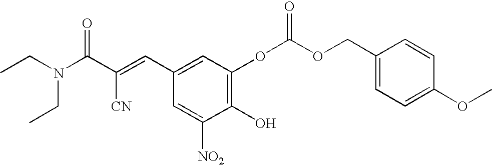 |
| | 1.27 (s (br), 6 H, N—CH₂—CH₃); 3.50 (s (br), 4 H, N—CH₂); 3.83 (s, 3 H, O—CH₃); |
| | 5.26 (s, 2 H, O—CH₂); 6.93 (d, J = 8.90 Hz, 2 H, aromat.); 7.39 (d, J = 8.69 Hz, |
| | 2 H, aromat.); 7.58 (s, 1 H, CH═C); 8.16 (d, J = 2.29 Hz, 1 H, aromat.); 8.49 (d, J = |
| | 2.29 Hz, 1 H, aromat.); 10.95 (s (br), 0.8 H, OH) |
| 82 | 2-[5-((E)-2-Cyano-2-diethylcarbamoyl-vinyl)-2-hydroxy-3-nitro- |
| | phenoxycarbonyloxy]-pentanoic acid methyl ester |
| | 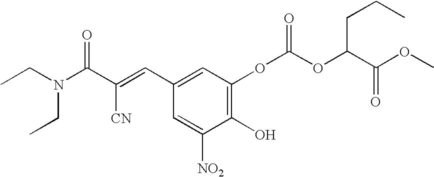 |
| 83 | Carbonic acid but-3-ynyl ester 5-((E)-2-cyano-2-diethylcarbamoyl-vinyl)-2- |
| | hydroxy-3-nitro-phenyl ester |
| | 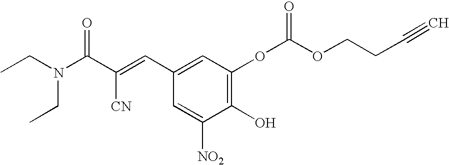 |
| | 1.27 (s (br), 6 H, N—CH₂—CH₃); 2.08 (t, J = 2.80 Hz, 1 H, CH≡C); 2.70 (dt, |
| | J = 2.54/6.86 Hz, 2 H, O—CH₂—CH₂); 3.50 (s (br), 4 H, N—CH₂); 4.42 (t, |
| | J = 6.86 Hz, 2 H, O—CH₂); 7.60 (s, 1 H, CH═C); 8.17 (d, J = 2.29 Hz, 1 H, |
| | aromat.); 8.49 (d, J = 2.29 Hz, 1 H, aromat.); 10.97 (s (br), 0.8 H, OH) |
| 84 | 2-[5-((E)-2-Cyano-2-diethylcarbamoyl-vinyl)-2-hydroxy-3-nitro- |
| | phenoxycarbonyloxy]-2-methyl-butyric acid methyl ester |
| | 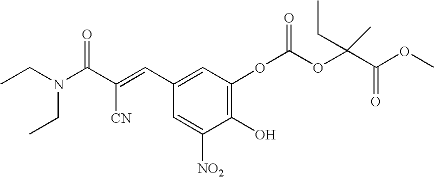 |
| 85 | Carbonic acid but-3-ynyl ester 2-but-3-ynyloxycarbonyloxy-4-((E)-2-cyano-2- |
| | diethylcarbamoyl-vinyl)-6-nitro-phenyl ester |
| | 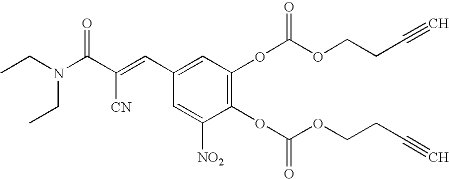 |
| | 1.25 (s (br), 6 H, N—CH₂—CH₃); 2.08 (t, J = 2.55 Hz, 1 H, CH≡C); 2.10 (t, J = |
| | 2.54 Hz, 1 H, CH≡C); 2.68 (m, 4 H, O—CH₂—CH₂); 3.51 (s (br), 4 H, N—CH₂); 4.42 |
| | (t, J = 6.61 Hz, 2 H, O—CH₂); 4.43 (t, J = 6.68 Hz, 2 H, O—CH₂); 7.63 (s, 1 H, |
| | CH═C); 8.22 (d, J = 2.29 Hz, 1 H, aromat.); 8.41 (d, J = 2.04 Hz, 1 H, aromat.) |
| 86 | 2-[5-((E)-2-Cyano-2-diethylcarbamoyl-vinyl)-2-hydroxy-3-nitro- |
| | phenoxycarbonyloxy]-3-methyl-butyric acid methyl ester |
| | 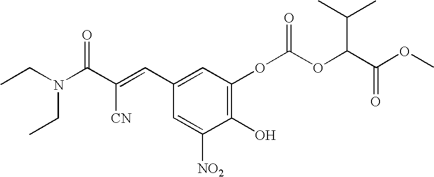 |
| 87 | Carbonic acid benzyl ester 2-benzyloxycarbonyloxy-4-((E)-2-cyano-2- |
| | diethylcarbamoyl-vinyl)-6-nitro-phenyl ester |
| | 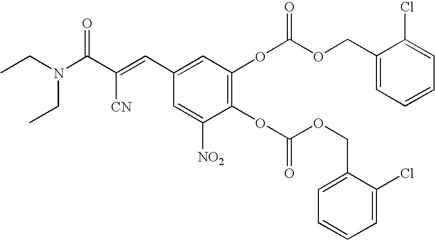 |
| | 1.27 (s (br), 6 H, N—CH₂—CH₃); 3.50 (s (br), 4 H, N—CH₂); 5.41 (s, 2 H, O—CH₂); |
| | 5.43 (s, 2 H, O—CH₂); 7.28-7.33 (m, 4 H, aromat.); 7.39-7.43 (m, 2 H, aromat.); |
| | 7.43-7.50 (m, 2 H, aromat.); 7.63 (s, 1 H, CH═C); 8.21 (d, J = 2.29 Hz, 1 H, |
| | aromat.); 8.42 (d, J = 2.29 Hz, 1 H, aromat.) |
| 88 | 2-[5-((E)-2-Cyano-2-diethylcarbamoyl-vinyl)-2-hydroxy-3-nitro- |
| | phenoxycarbonyloxy]-malonic acid dimethyl ester |
| | 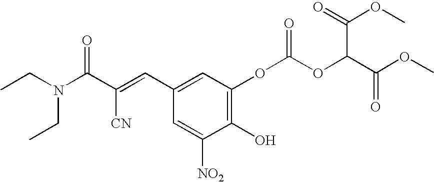 |
| 89 | 2-[5-((E)-2-Cyano-2-diethylcarbamoyl-vinyl)-2-hydroxy-3-nitro- |
| | phenoxycarbonyloxy]-succinic acid dimethyl ester |
| | 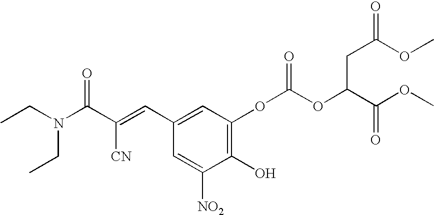 |
| 90 | Carbonic acid 5-((E)-2-cyano-2-diethylcarbamoyl-vinyl)-2-hydroxy-3-nitro-phenyl |
| | ester 4-methyl-2-oxo-tetrahydro-pyran-4-yl ester |
| | 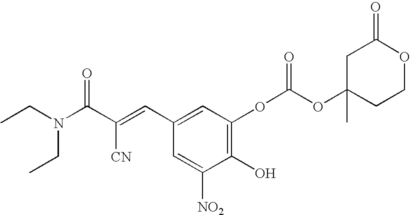 |
| 91 | Carbonic acid 5-((E)-2-cyano-2-diethylcarbamoyl-vinyl)-2-hydroxy-3-nitro-phenyl |
| | ester 1-ethyl-3-hydroxy-propyl ester |
| | 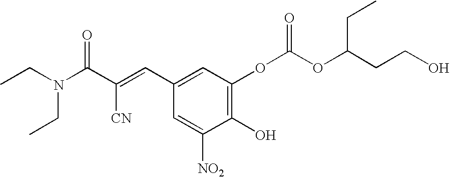 |
| 92 | 2-[5-((E)-2-Cyano-2-diethylcarbamoyl-vinyl)-2-hydroxy-3-nitro- |
| | phenoxycarbonyloxy]-cyclopentanecarboxylic acid methyl ester |
| | 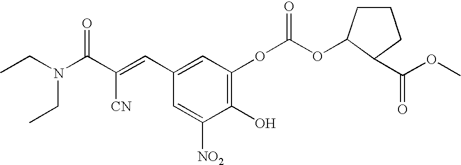 |
| 93 | 3-[5-((E)-2-Cyano-2-diethylcarbamoyl-vinyl)-2-hydroxy-3-nitro- |
| | phenoxycarbonyloxy]-3-methoxycarbonyl-pentanedioic acid dimethyl ester |
| | 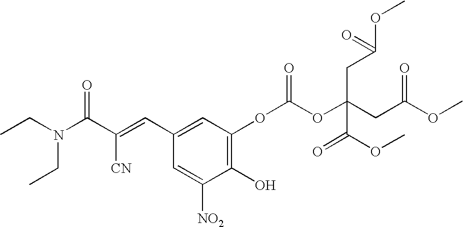 |
| 94 | Carbonic acid 5-((E)-2-cyano-2-diethylcarbamoyl-vinyl)-2-hydroxy-3-nitro-phenyl |
| | ester 5-methyl-tetrahydro-furan-2-ylmethyl ester |
| | 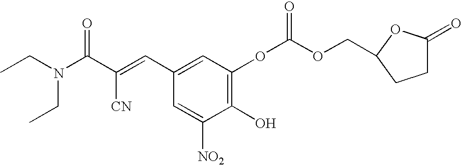 |
| 95 | 2-[5-((E)-2-Cyano-2-diethylcarbamoyl-vinyl)-2-hydroxy-3-nitro- |
| | phenoxycarbonyloxy]-3-hydroxy-succinic acid dimethyl ester |
| | 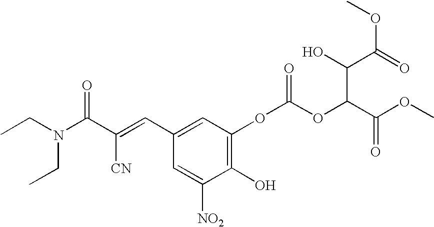 |
| 96 | Carbonic acid 5-((E)-2-cyano-2-diethylcarbamoyl-vinyl)-2-hydroxy-3-nitro-phenyl |
| | ester 5-oxo-2,5-dihydro-furan-2-ylmethyl ester |
| | 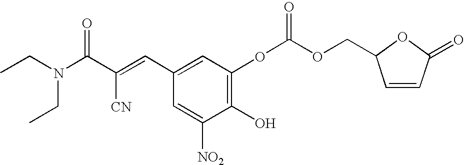 |
| | 1.26 (s (br), 6 H, N—CH₂—CH₃); 3.49 (s (br), 2 H, N—CH₂); 3.51 (s (br), 2 H, |
| | N—CH₂); 4.56 (m, 2 H, O—CH₂); 5.33 (m, 1 H, O—CH); 6.30 (m, J = 2.04 Hz, 1 H, |
| | CO—CH═CH); 7.51 (dd, J = 2.04/5.84 Hz, 1 H, O═C—CH═CH); 7.59 (s, 1 H, |
| | CH═C); 8.14 (d, J = 2.29 Hz, 1 H, aromat.); 8.49 (d, J = 2.28 Hz, 1 H, aromat.); |
| | 10.95 (s (br), 0.9 H, OH) |
| 97 | Carbonic acid 5-((E)-2-cyano-2-diethylcarbamoyl-vinyl)-2-hydroxy-3-nitro-phenyl |
| | ester 2-oxo-[1,3]dioxolan-4-ylmethyl ester |
| | 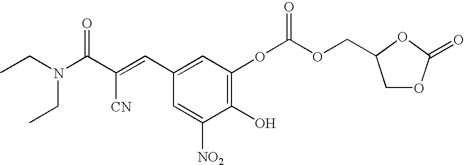 |
| | 1.24 (s (br), 3 H, N—CH₂—CH₃); 1.31 (s (br), 3 H, N—CH₂—CH₃); 3.50 (s (br), 4 H, |
| | N—CH₂); 4.42-4.50 (m, 2 H, Heterocyclus-CH₂); 4.60-4.67 (m, 2 H, O—CH₂); 5.01- |
| | 5.06 (1 H, m, CH); 7.60 (s, 1 H, CH═C); 8.18 (d, J = 2.29 Hz, 1 H, aromat.); 8.50 |
| | (d, J = 2.29 Hz, 1 H, aromat); 10.98 (s (br), 0.9 H, OH) |
| 98 | Carbonic acid 5-((E)-2-cyano-2-diethylcarbamoyl-vinyl)-2-hydroxy-3-nitro-phenyl |
| | ester 2-fluoro-benzyl ester |
| | 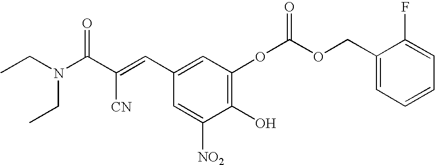 |
| 99 | 2-[5-((E)-2-Cyano-2-diethylcarbamoyl-vinyl)-2-hydroxy-3-nitro- |
| | phenoxycarbonyloxy]-2-ethyl-3-methyl-butyric acid methyl ester |
| | 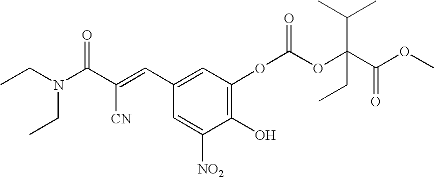 |
| 100 | Carbonic acid 5-((Z)-2-cyano-2-diethylcarbamoyl-vinyl)-2-hydroxy-3-nitro-phenyl |
| | ester ethyl ester |
| | 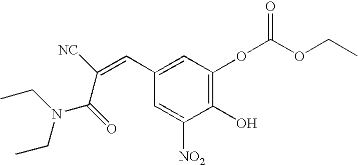 |
| 101 | 2-[5-((E)-2-Cyano-2-diethylcarbamoyl-vinyl)-2-hydroxy-3-nitro- |
| | phenoxycarbonyloxy]-ethanesulfonic acid |
| | 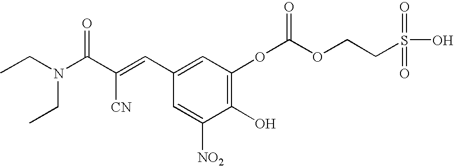 |
| 102 | 3-{4-[5-((E)-2-Cyano-2-diethylcarbamoyl-vinyl)-2-hydroxy-3-nitro- |
| | phenoxycarbonyloxy]-phenyl}-propionic acid |
| | 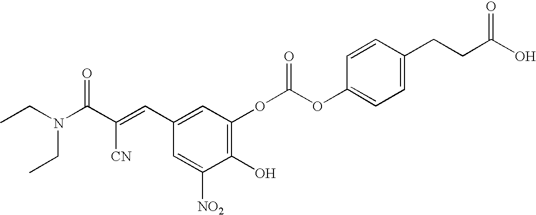 |
| 103 | 2-[5-((E)-2-Cyano-2-diethylcarbamoyl-vinyl)-2-hydroxy-3-nitro- |
| | phenoxycarbonyloxy]-benzoic acid octyl ester |
| | 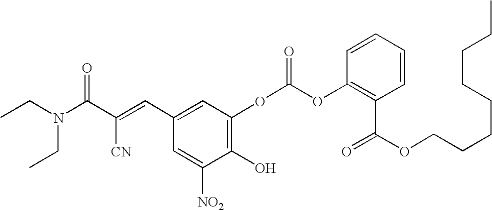 |
| 104 | (E)-3-{4-[5-((E)-2-Cyano-2-diethylcarbamoyl-vinyl)-2-hydroxy-3-nitro- |
| | phenoxycarbonyloxy]-phenyl}-acrylic acid |
| | 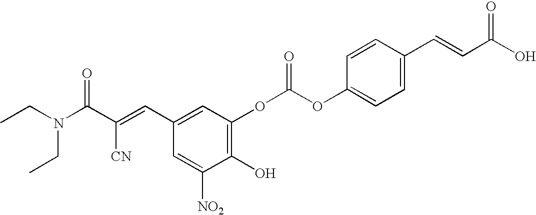 |
| 105 | 3-[2-(2-{2-[5-((E)-2-Cyano-2-diethylcarbamoyl-vinyl)-2-hydroxy-3-nitro- |
| | phenoxycarbonyloxy]-ethoxy}-ethoxy)-ethoxy]-propionic acid tert-butyl ester |
| | 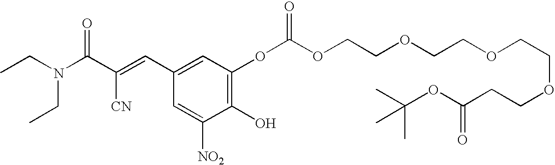 |
| 106 | Carbonic acid 2-amino-butyl ester 5-((E)-2-cyano-2-diethylcarbamoyl-vinyl)-2- |
| | hydroxy-3-nitro-phenyl ester |
| | 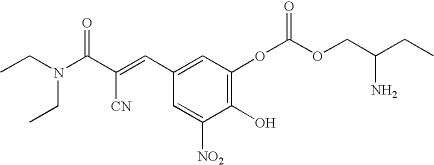 |
| 107 | Carbonic acid 1-carbamoylmethyl-propyl ester 5-((E)-2-cyano-2- |
| | diethylcarbamoyl-vinyl)-2-hydroxy-3-nitro-phenyl ester |
| | 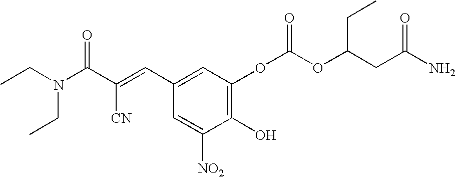 |
| 108 | Carbonic acid 5-((E)-2-cyano-2-diethylcarbamoyl-vinyl)-2-hydroxy-3-nitro-phenyl |
| | ester 2-{2-[2-(2-hydroxy-ethoxy)-ethoxy]-ethoxy}-ethyl ester |
| | 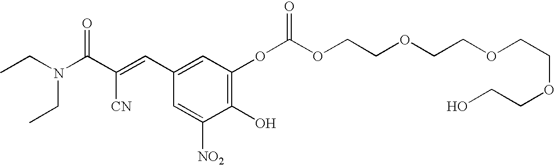 |
| 109 | Carbonic acid 5-((E)-2-cyano-2-diethylcarbamoyl-vinyl)-2-hydroxy-3-nitro-phenyl |
| | ester 2-(2-propoxy-ethoxy)-ethyl ester |
| | 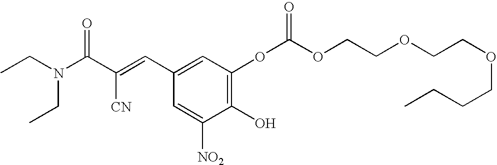 |
| 110 | Carbonic acid 5-((E)-2-cyano-2-diethylcarbamoyl-vinyl)-2-hydroxy-3-nitro-phenyl |
| | ester methyl ester |
| | 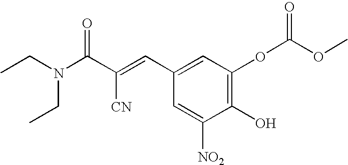 |
| | 1.30 (s (br), 6 H, N—CH₂—CH₃); 3.50 (s (br), 4 H, N—CH₂); 3.98 (s, 3 H, O—CH₃); |
| | 7.60 (s, 1 H, CH═C); 8.17 (d, J = 2.29 Hz, 1 H, aromat.); 8.48 (d, J = 2.29 Hz, |
| | 1 H, aromat.); 10.96 (s (br), 1 H, OH) |
| 111 | Carbonic acid 5-((E)-2-cyano-2-dipropylcarbamoyl-vinyl)-2-hydroxy-3-nitro- |
| | phenyl ester pentyl ester |
| | 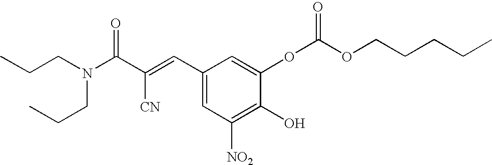 |
| 112 | Carbonic acid 5-((E)-2-cyano-2-diethylcarbamoyl-vinyl)-2-hydroxy-3-nitro-phenyl |
| | ester hexyl ester |
| | 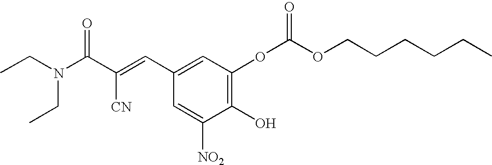 |
| 113 | Carbonic acid 5-((E)-2-cyano-2-dipentylcarbamoyl-vinyl)-2-hydroxy-3-nitro- |
| | phenyl ester 1-methyl-pentyl ester |
| | 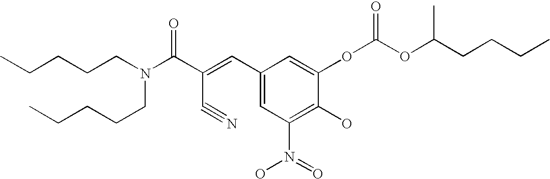 |
| 114 | Carbonic acid 5-((E)-2-cyano-2-diethylcarbamoyl-vinyl)-2-hydroxy-3-nitro-phenyl |
| | ester heptyl ester |
| | 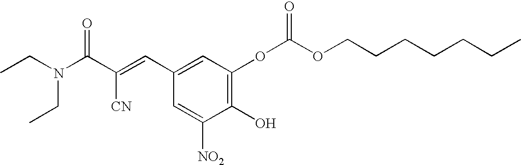 |
| 115 | Carbonic acid 5-((E)-2-cyano-2-diethylcarbamoyl-vinyl)-2-hydroxy-3-nitro-phenyl |
| | ester 2-methyl-pentyl ester |
| | 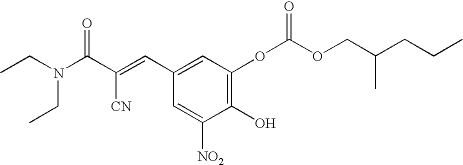 |
| 116 | Carbonic acid 5-((E)-2-cyano-2-diethylcarbamoyl-vinyl)-2-hydroxy-3-nitro-phenyl |
| | ester 2-methyl-butyl ester |
| | 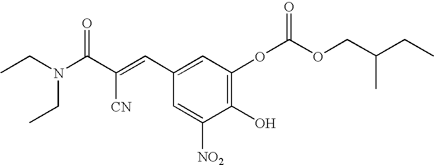 |
| 117 | Carbonic acid 5-((E)-2-cyano-2-diethylcarbamoyl-vinyl)-2-hydroxy-3-nitro-phenyl |
| | ester 3-methyl-pentyl ester |
| | 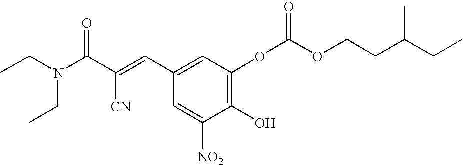 |
| 118 | Carbonic acid 5-((E)-2-cyano-2-diethylcarbamoyl-vinyl)-2-hydroxy-3-nitro-phenyl |
| | ester 2-methyl-butyl ester |
| | 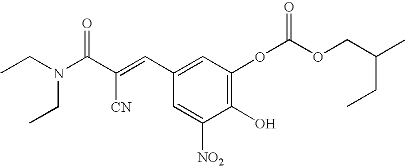 |
| 119 | Carbonic acid 5-((E)-2-cyano-2-diethylcarbamoyl-vinyl)-2-hydroxy-3-nitro-phenyl |
| | ester 4-methyl-pentyl ester |
| | 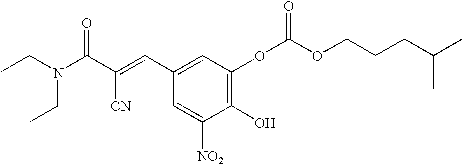 |
| 120 | Carbonic acid 5-((Z)-2-cyano-2-diethylcarbamoyl-vinyl)-2-hydroxy-3-nitro-phenyl |
| | ester 2-ethyl-hexyl ester |
| | 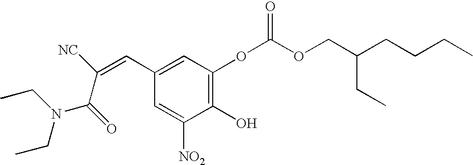 |
| 121 | Carbonic acid 5-((E)-2-cyano-2-diethylcarbamoyl-vinyl)-2-hydroxy-3-nitro-phenyl |
| | ester 1-ethyl-butyl ester |
| | 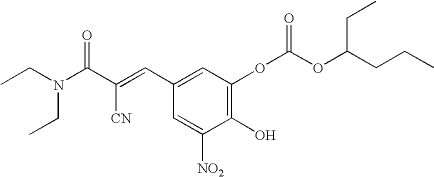 |
| | 0.97 (t, J = 7.12 Hz, 3 H, CH₃); 1.01 (t, J = 7.38 Hz, 3 H, CH₃); 1.28 (s (br), 6 H, |
| | N—CH₂—CH₃); 1.38-1.54 (m, 2 H, CH₂); 1.59-1.67 (m, 1 H, CH—CH₂); 1.69- |
| | 1.77 (m, 2 + 1 H, CH₂+ CH—CH₂); 3.50 (s (br), 4 H, N—CH₂); 4.80 (m, 1 H, O—CH); |
| | 7.59 (s, 1 H, CH═C); 8.15 (d, J = 2.29 Hz, 1 H, aromat.); 8.49 (d, J = 2.29 Hz, |
| | 1 H, aromat.); 10.97 (s, 1 H, OH) |
| 122 | Carbonic acid 5-((E)-2-cyano-2-diethylcarbamoyl-vinyl)-2-hydroxy-3- |
| | nitro-phenyl ester 1-methyl-pentyl ester |
| | 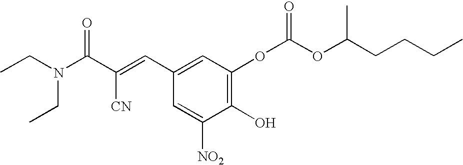 |
| 123 | Carbonic acid 5-((E)-2-cyano-2-diethylcarbamoyl-vinyl)-2-hydroxy-3-nitro-phenyl |
| | ester 2-ethyl-butyl ester |
| | 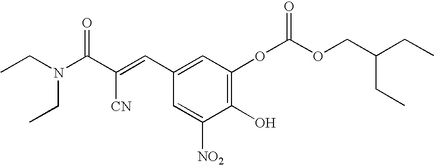 |
| 124 | Carbonic acid 5-((E)-2-cyano-2-diethylcarbamoyl-vinyl)-2-hydroxy-3-nitro-phenyl |
| | ester 1,1-dimethyl-butyl ester |
| | 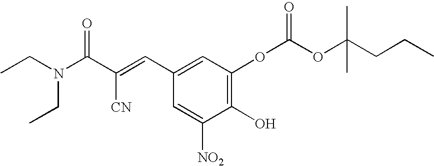 |
| 125 | Carbonic acid 5-((E)-2-cyano-2-diethylcarbamoyl-vinyl)-2-hydroxy-3-nitro-phenyl |
| | ester 3-methyl-pentyl ester |
| | 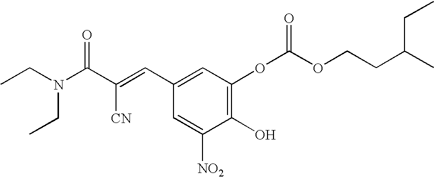 |
| 126 | Carbonic acid 5-((E)-2-cyano-2-diethylcarbamoyl-vinyl)-2-hydroxy-3-nitro-phenyl |
| | ester 2,2-dimethyl-butyl ester |
| | 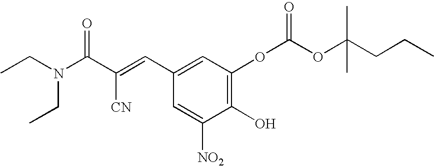 |
| 127 | 2-[5-((E)-2-Cyano-2-diethylcarbamoyl-vinyl)-2-hydroxy-3-nitro- |
| | phenoxycarbonyloxymethyl]-pyrrolidine-1-carboxylic acid tert-butyl ester |
| | 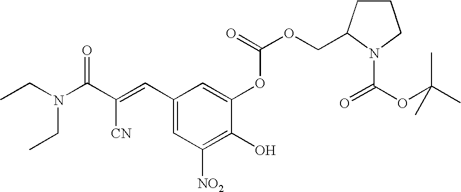 |
| | 1.27 (s (br), 6 H, N—CH₂—CH₃); 1.48 (s, 9 H, C(CH₃)₃); 1.87-2.07 (m, 4 H, CH₂); |
| | 3.41 (s (br), N—CH₂); 3.50 (s (br), 4 H, N—CH₂ [Entacapon]); 4.07-4.29 (m, 1 H, |
| | N—CH); 4.73 (s, 2 H, O—CH₂); 7.59 (s, 1 H, CH═C); 8.14 (s (br), 1 H, aromat.); 8.48 (d, |
| | J = 2.29 Hz, 1 H, aromat); 10.94 (s (br), 0.9 H, OH) |
| 128 | 3-[5-((E)-2-Cyano-2-diethylcarbamoyl-vinyl)-2-hydroxy-3-nitro- |
| | phenoxycarbonyloxy]-2,2-dimethyl-propionic acid methyl ester |
| | 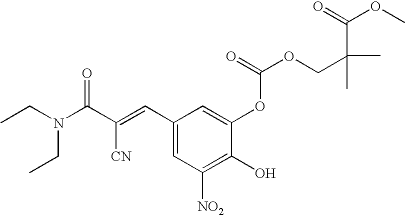 |
| | 1.27 (s (br), 6 H, N—CH2—CH3); 1.31 (s, 6 H, C(CH3)2); 3.50 (s (br), 4 H, N—CH2); |
| | 3.75 (s, 3 H, O—CH3); 4.36 (s, 2 H, O—CH2); 7.59 (s, 1 H, CH═C); 8.14 (d, |
| | J = 2.29 Hz, 1 H, aromat.); 8.50 (d, J = 2.29 Hz, 1 H, aromat); 10.96 (s (br), 1 H, |
| | OH) |
| 129 | Carbonic acid 5-((E)-2-cyano-2-diethylcarbamoyl-vinyl)-2-hydroxy-3-nitro-phenyl |
| | ester furan-3-ylmethyl ester |
| | 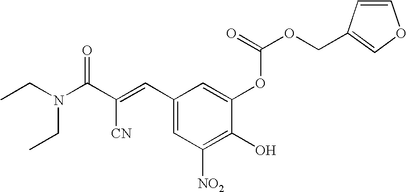 |
| | 1.28 (s (br), 6 H, N—CH2—CH3); 3.50 (s (br), 4 H, N—CH2); 5.21 (s, 2 H, O—CH2); |
| | 6.53 (d, J = 1.25 Hz, 1 H, O—CH═CH); 7.45 (t, J = 1.76 Hz, 1 H, O—CH═C); 7.58 (d, |
| | J = 0.75 Hz, 1 H, O—CH═CH); 7.59 (s, 1 H, CH═C); 8.15 (d, J = 2.26 Hz, 1 H, |
| | aromat.); 8.49 (d, J = 2.01 Hz, 1 H, aromat.); 10.95 (s (br), 0.9 H, OH) |
| 130 | Carbonic acid 5-((E)-2-cyano-2-diethylcarbamoyl-vinyl)-2-hydroxy-3-nitro-phenyl |
| | ester tetrahydro-furan-2-ylmethyl ester |
| | 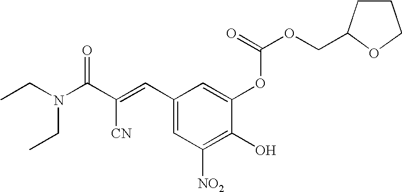 |
| | 1.29 (s (br), 3 H, N—CH2—CH3); 1.69-1.77 (m, 1 H, CH—CH2); 1.91-2.01 (m, 2 H, |
| | CH2); 2.04-2.12 (m, 1 H, CH—CH2); 3.50 (s (br), 4 H, N—CH2); 3.84 (m, 1 H, |
| | O—CH2—CH2); 3.93 (m, 1 H, O—CH2—CH2); 4.22-4.29 (m, 2 H, O—CH2—CH); 4.32- |
| | 4.39 (1 H, m, CH); 7.59 (s, 1 H, CH═C); 8.14 (d, J = 2.04 Hz, 1 H, aromat.); 8.50 (d, |
| | J = 2.29 Hz, 1 H, aromat); 10.96 (s (br), 0.9 H, OH) |
| 131 | Carbonic acid benzo[1,3]dioxol-5-ylmethyl ester 5-((E)-2-cyano-2- |
| | diethylcarbamoyl-vinyl)-2-hydroxy-3-nitro-phenyl ester |
| | 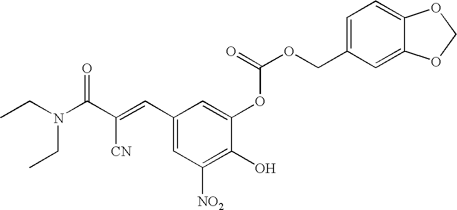 |
| | 1.28 (s (br), 6 H, N—CH2—CH3); 3.50 (s (br), 4 H, N—CH2); 5.22 (s, 2 H, O—CH2); |
| | 6.00 (s, 2 H, O—CH2—O); 6.83 (dd, J = 1.27/8.39 Hz, 1 H, aromat.); 6.93 (dd, |
| | J = 1.78/7.78 Hz, 1 H, aromat.); 6.94 (s, 1 H, aromat.); 7.58 (s, 1 H, CH═C); 8.14 (d, |
| | J = 2.03 Hz, 1 H, aromat.); 8.49 (d, J = 2.29 Hz, 1 H, aromat.); 10.95 (s (br), 1 H, |
| | OH) |
| 132 | Carbonic acid 5-((E)-2-cyano-2-diethylcarbamoyl-vinyl)-2-hydroxy-3-nitro-phenyl |
| | ester 4-methyl-cyclohexyl ester |
| | 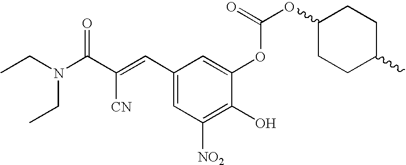 |
| | 0.92 (d, J = 6.61 Hz, 3 H, CH—CH3); 1.01-1.12 (m, 2 H, cyclohexyl-CH2); 1.27 (s |
| | (br), 6 H, N—CH2—CH3); 1.38-1.46 (m, 1 H, CH—CH3); 1.48-1.55 (m, 2 H, |
| | cyclohexyl-CH2); 1.79 (s (br), 1 H, cyclohexyl-CH2); 1.82 (s (br), 1 H, cyclohexyl- |
| | CH2); 2.02-2.17 (m, 2 H, cyclohexyl-CH2); 3.50 (s (br), 4 H, N—CH2); 4.63-4.70 |
| | (m, 1 H, O—CH); 7.59 (s, 1 H, CH═C); 8.15 (d, J = 2.29 Hz, 1 H, aromat.); 8.48 (d, |
| | J = 1.78 Hz, 1 H, aromat.); 10.97 (s (br), 1 H, OH) |
| 133 | Carbonic acid 5-((E)-2-cyano-2-diethylcarbamoyl-vinyl)-2-hydroxy-3-nitro-phenyl |
| | ester 2-methyl-cyclohexyl ester |
| | 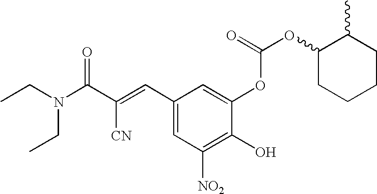 |
| | 1.05 (d, J = 6.61 Hz, 3 H, CH—CH3); 1.07-1.16 (m, 2 H, cyclohexyl-CH2); 1.27 (s |
| | (br), 6 H, N—CH2—CH3); 1.43-1.53 (m, 2 H, cyclohexyl-CH2); 1.64-1.74 (m, 2 H, |
| | cyclohexyl-CH2); 1.80-1.84 (m, 2 H, cyclohexyl-CH2); 2.14 (m, 1 H, CH—CH3); |
| | 3.50 (s (br), 4 H, N—CH2); 4.40 + 4.93 (s + dt, J = 4.32/10.43 Hz, 1 H, O—CH); 7.59 |
| | (s, 1 H, CH═C); 8.15 (d, J = 2.29 Hz, 1 H, aromat.); 8.49 (d, J = 2.29 Hz, 1 H, |
| | aromat.); 10.97 (s (br), 0.9 H, OH) |
| 134 | Carbonic acid 5-((E)-2-cyano-2-diethylcarbamoyl-vinyl)-2-hydroxy-3-nitro-phenyl |
| | ester 3,5-dimethyl-cyclohexyl ester |
| | 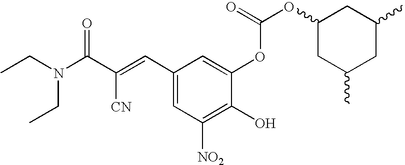 |
| | 0.93-0.98 (d, J = 6.62 Hz, 6 H, CH—CH3); 1.11 (q, 2 H, cyclo-hexyl-CH2); 1.28 (s |
| | (br), 6 H, N—CH2—CH3); 1.40-1.52 (m, 2 H, cyclohexyl-CH2); 1.60-1.68 (m, 2 H, |
| | cyclohexyl-CH2); 2.04-2.15 (m, 2 H, cyclohexyl-CH2); 3.50 (s (br), 4 H, N—CH2); |
| | 4.68-5.13 (m, 1 H, O—CH); 7.59 (s, 1 H, CH═C); 8.15 (d, J = 2.29 Hz, 1 H, |
| | aromat.); 8.48 (d, J = 2.28 Hz, 1 H, aromat.); 10.97 (s (br), 0.9 H, OH) |
| 135 | Carbonic acid 5-((E)-2-cyano-2-diethylcarbamoyl-vinyl)-2-hydroxy-3-nitro-phenyl |
| | ester 2,5-dimethyl-cyclohexyl ester |
| | 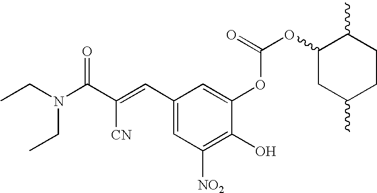 |
| | 0.97 + 0.98 + 1.04 (3 m, 6 H, CH—CH3); 1.08-1.48 (m, 10 H, cyclo-hexyl-CH2 + |
| | N—CH2—CH3); 1.60-2.06 (m, 4 H, cyclohexyl-CH2); 3.50 (s (br), 4 H, N—CH2); |
| | 4.40 + 4.72 + 4.82 (3 m, 1 H, O—CH); 7.59 (s, 1 H, CH═C); 8.15 (d, 1 H, aromat.); |
| | 8.49 (d, aromat.); 10.97 (s (br), 0.9 H, OH) |
| 136 | Carbonic acid bicyclo[2.2.1]hept-2-yl ester 5-((E)-2-cyano-2-diethylcarbamoyl- |
| | vinyl)-2-hydroxy-3-nitro-phenyl ester |
| | 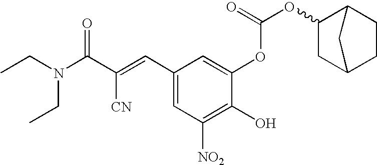 |
| | 1.14 (m, 2 H, CH—CH2—CH); 1.27 (s (br), 6 H, N—CH2—CH3); 1.38-1.51 (m, 2 H, |
| | cyclohexyl-CH2); 1.60-1.69 (m, 2 H, cyclohexyl-CH2); 1.80-1.92 und 2.05- |
| | 2.14 (m, 2 H, cyclohexyl-CH2); 2.29 (t, J = 4.07 Hz, 0.3 H, CH—CH3); 2.37 (t, |
| | J = 3.82 Hz, 0.7 H, CH—CH3); 2.51 (d, J = 4.83 Hz, 0.7 H, CH—CH3); 2.64 (m, 0.3 H, |
| | CH—CH3); 3.50 (s (br), 4 H, N—CH2); 5.01 und 4.49 (m, 1 H, O—CH); 7.59 (s, 1 H, |
| | CH═C); 8.16 (d, J = 1.78 Hz, 1 H, aromat.); 8.48 (d, J = 2.29 Hz, 1 H, aromat.); |
| | 10.97 (s (br), 0.9 H, OH) |
| 137 | Carbonic acid 5-((E)-2-cyano-2-diethylcarbamoyl-vinyl)-2-hydroxy-3-nitro-phenyl |
| | ester 1-phenyl-ethyl ester |
| | 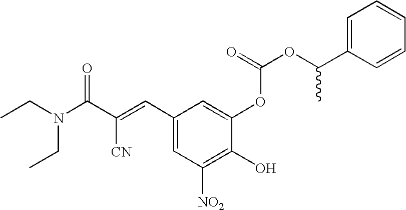 |
| | 1.26 (s (br), 6 H, N—CH2—CH3); 1.72 (d, J = 6.62 Hz, 3 H, CH—CH3); 3.49 (s (br), |
| | 4 H, N—CH2); 5.85 (q, J = 6.61 Hz, 1 H, O—CH); 7.34-7.45 (m, 5 H, aromat.); 7.57 |
| | (s, 1 H, CH═C); 8.11 (d, J = 2.03 Hz, 1 H, aromat.); 8.49 (d, J = 2.04 Hz, 1 H, |
| | aromat.); 10.91 (s, 1 H, OH) |
| 138 | Carbonic acid 5-((E)-2-cyano-2-diethylcarbamoyl-vinyl)-2-hydroxy-3-nitro-phenyl |
| | ester 1-phenyl-propyl ester |
| | 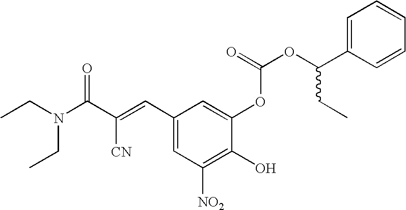 |
| | 0.99 (t, J = 7.63 Hz, 3 H, CH3); 1.25 (s (br), 6 H, N—CH2—CH3); 1.96 (m, |
| | J = 6.68/7.38 Hz, 1 H, CH—CH2); 2.12 (m, J = 6.61/7.38 Hz 1 H, CH—CH2); 3.49 (s, |
| | (br), 4 H, N—CH2); 5.61 (t, J = 6.61 Hz, 1 H, O—CH); 7.33-7.40 (m, 5 H, aromat.); |
| | 7.56 (s, 1 H, CH═C); 8.09 (d, J = 2.03 Hz, 1 H, aromat.); 8.49 (d, J = 2.29 Hz, 1 H, |
| | aromat.); 10.89 (s, 0.9 H, OH) |
| 139 | Carbonic acid 5-((E)-2-cyano-2-diethylcarbamoyl-vinyl)-2-hydroxy-3-nitro-phenyl |
| | ester 1-methyl-2-phenyl-ethyl ester |
| | 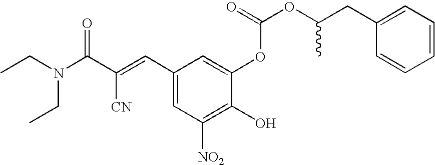 |
| | 1.28 (s (br), 6 H, N—CH2—CH3); 1.40 (d, J = 6.36 Hz, 3 H, CH3); 2.91 (dd, |
| | J = 6.61/7.12 Hz, 1 H, CH—CH2); 3.08 (dd, J = 6.61/7.12 Hz, 1 H, CH—CH2); 3.50 (s |
| | (br), 4 H, N—CH2); 5.10 (m, J = 6.35 Hz, 1 H, O—CH); 7.25 (m, 3 H, aromat.); 7.33 |
| | (m, 2 H, aromat.); 7.58 (s, 1 H, CH═C); 8.08 (d, J = 2.04 Hz, 1 H, aromat.); 8.48 (d, J = |
| | 2.29 Hz, 1 H, aromat.); 10.89 (s, 1 H, OH) |
| 140 | Carbonic acid 5-((E)-2-cyano-2-diethylcarbamoyl-vinyl)-2-hydroxy-3-nitro-phenyl |
| | ester 1-phenyl-prop-2-ynyl ester |
| | 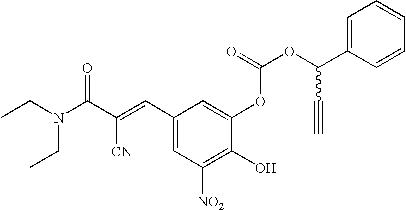 |
| | 1.26 (s (br), 6 H, N—CH2—CH3); 2.83 (s, 1 H, C≡CH); 3.49 (s (br), 4 H, N—CH2); 6.37 |
| | (d, J = 2.03 Hz, 1 H, O—CH); 7.44 (m, 3 H, aromat.); 7.57 (s, 1 H, CH═C); 7.61 (m, |
| | 2 H, aromat.); 8.13 (d, J = 2.29 Hz, 1 H, aromat.); 8.50 (d, J = 2.29 Hz, 1 H, |
| | aromat.); 10.92 (s, 0.9 H, OH) |
| 141 | Carbonic acid 5-((E)-2-cyano-2-diethylcarbamoyl-vinyl)-2-hydroxy-3-nitro-phenyl |
| | ester 2-(2-methoxy-phenyl)-ethyl ester |
| | 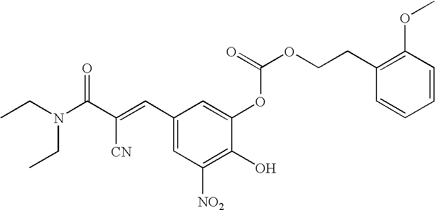 |
| | 1.28 (s (br), 6 H, N—CH2—CH3); 3.10 (t, J = 7.12 Hz, 2 H, O—CH2—CH2); 3.50 (s (br), |
| | 4 H, N—CH2); 3.85 (s, 3 H, O—CH3); 4.50 (t, J = 7.12 Hz, 2 H, O—CH2); 6.90 (m, 2 H, |
| | aromat.); 7.18 (m, 1 H, aromat.); 7.24 (m, 1 H, aromat.); 7.58 (s, 1 H, CH═C); 8.11 |
| | (d, J = 2.29 Hz, 1 H, aromat.); 8.48 (d, J = 2.03 Hz, 1 H, aromat.); 10.92 (s, 1 H, OH) |
| 142 | Carbonic acid 5-((E)-2-cyano-2-diethylcarbamoyl-vinyl)-2-hydroxy-3-nitro-phenyl |
| | ester 2-(3-methoxy-phenyl)-ethyl ester |
| | 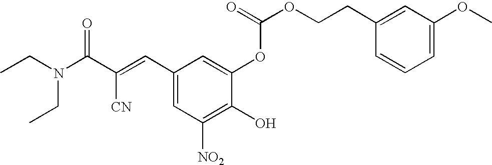 |
| | 1.20 (s (br), 6 H, N—CH2—CH3); 2.99 (t, J = 7.12 Hz, 2 H, O—CH2—CH2); 3.44 (s (br), |
| | 4 H, N—CH2); 3.84 (s, 3 H, O—CH3); 4.44 (t, J = 7.12 Hz, 2 H, O—CH2); 6.72-6.78 |
| | (m, 3 H, aromat.); 7.17 (m, 1 H, aromat.); 7.52 (s, 1 H, CH═C); 8.05 (d, J = 2.29 Hz, |
| | 1 H, aromat.); 8.42 (d, J = 2.03 Hz, 1 H, aromat.); 10.85 (s, 1 H, OH) |
| 143 | Carbonic acid 5-((E)-2-cyano-2-diethylcarbamoyl-vinyl)-2-hydroxy-3-nitro-phenyl |
| | ester 2-(4-methoxy-phenyl)-1-methyl-ethyl ester |
| | 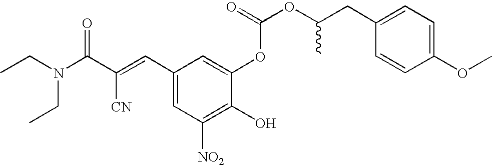 |
| | 1.27 (s (br), 6 H, N—CH2—CH3); 3.02 (t, J = 7.12 Hz, 2 H, O—CH2—CH2); 3.50 (s (br), |
| | 4 H, N—CH2); 3.80 (s, 3 H, O—CH3); 4.47 (t, J = 7.12 Hz, 2 H, O—CH2); 6.88 (d, |
| | J = 8.65 Hz, 2 H, aromat.); 7.17 (d, J = 8.65 Hz, 2 H, aromat.); 7.68 (s, 1 H, CH═C); |
| | 8.12 (d, J = 2.29 Hz, 1 H, aromat.); 8.48 (d, J = 2.29 Hz, 1 H, aromat.); 10.93 (s, |
| | 0.9 H, OH) |
| 144 | Carbonic acid adamantan-1-yl ester 5-((E)-2-cyano-2-diethylcarbamoyl-vinyl)-2- |
| | hydroxy-3-nitro-phenyl ester |
| | 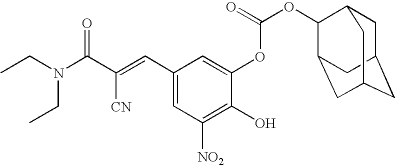 |
| | 1.28 (s (br), 6 H, N—CH2—CH3); 1.63-2.22 (m, 14 H, adamantyl); 3.50 (s (br), 4 H, |
| | N—CH2); 4.94 (s, 1 H, O—CH); 7.59 (s, 1 H, CH═C); 8.16 (d, J = 2.20 Hz, 1 H, |
| | aromat.); 8.49 (d, J = 2.20 Hz, 1 H, aromat.); 10.97 (s, 1 H, OH) |
| 145 | 3-[5-((E)-2-Cyano-2-diethylcarbamoyl-vinyl)-2-hydroxy-3-nitro- |
| | phenoxycarbonyloxy]-butyric acid tert-butyl ester |
| | 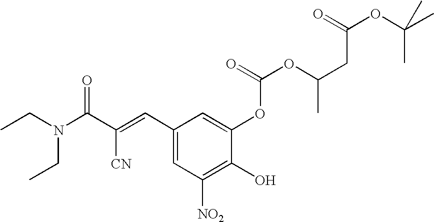 |
| | 1.28 (s (b), 6 H, N—CH2—CH3); 1.46 (d, J = 6.36 Hz, 3 H, C—CH3); 1.48 (s, 9 H, |
| | C(CH3)3); 2.55 (dd, J = 5.85/15.76 Hz, 1 H, CH—CH2); 2.74 (dd, J = 7.37/15.77 |
| | Hz, 1 H, CH—CH2); 3.51 (s (br), 4 H, N—CH2); 5.27 (m, 1 H, O—CH); 7.59 (s, 1 H, |
| | CH═C); 8.13 (d, J = 2.29 Hz, 1 H, aromat.); 8.50 (d, J = 2.29 Hz, 1 H, aromat.); |
| | 10.95 (s (br), 0.9 H, OH) |
| 146 | [5-((E)-2-Cyano-2-diethylcarbamoyl-vinyl)-2-hydroxy-3-nitro- |
| | phenoxycarbonyloxy]-acetic acid |
| | 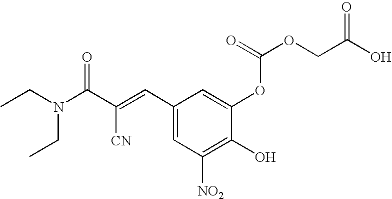 |
| | 1.24 + 1.32 (s (br), 6 H, N—CH2—CH3); 3.52 (s (br), 4 H, N—CH2); 4.81 (s, 2 H, |
| | O—CH2); 7.70 (s, 1 H, CH═C); 8.12 (d, J = 2.29 Hz, 1 H, aromat.); 8.62 (d, J = 2.04 Hz, |
| | 1 H, aromat.) |

### Methods

The following in vivo and in vitro tests have been carried out on selected compounds mentioned in Table 1 of the present invention:

i) Physicochemical Characterization
ia) Purity

The purity of all compounds is determined by reversed phase gradient HPLC method, column YMC Hydrosphere C18, 150 mm×4.0 mm, 3 μm, 12 nm, Eluent A: Water: trifluoroacetic acid (100:0.5 (v/v)), Eluent B: Acetonitrile: trifluoroacetic acid (100:0.5 (v/v)). Column temperature 40° C., Flow rate 1.4 mL/min, Detection wavelength: 220 nm

All compounds disclosed in table 1 have a purity of ≧95%.

ib) Stability at Different pH-Values, t_{1/2} [h]

The stability of compounds of the present invention is tested at pH 1 and at pH 7.4. The testing is carried out by the following HPLC method:

Chemical hydrolysis rates t_{1/2} [h] of selected compounds of formula I are determined in 0.1 N HCl (pH 1) and PBS buffer (pH 7.4) solution. Solutions are prepared by dissolving an appropriate amount of compound in 0.1 N HCl and PBS buffer solution. Samples were withdrawn at designated time intervals and analysed for remaining prodrug by HPLC at 37° C. over 12 h.

Reversed phase gradient HPLC method, column YMC Pro C8, 50 mm×4.0 mm, 3 μm, Eluent A: Water:trifluoroacetic acid (100:0.5 (v/v)), Eluent B: Acetonitrile:trifluoroacetic:acid (100:0.5 (v/v)). Column temperature 30° C., Flow rate 3.0 mL/min, Detection wavelength: 220 nm

For evaluation, the peak area of the respective compound from the first injection is defined as 100% relative concentration. The peak areas of all other subsequent injections are calculated in percentage relating to this standard. (relative concentration in %). The relative concentrations are plotted in a logarithmic scale (ln) against the injection times. Based on the assumption of a first order kinetic, the rate constant and half life times (t_{1/2} [h]) can be calculated.

The results of the study are summarized in Table 2.

TABLE 2

| Compound | t_{1/2} at pH 1 (h) | t_{1/2} at pH 7.4 (h) |
|---|---|---|
| entacapone¹ | stable | stable |
| 1 | 6.8 | 11.6 |
| 7 | 2.7 | 23.1 |
| 10 | 38.5 | 38.5 |
| 11 | 3.0 | 4.1 |
| 12 | <1 | 2.8 |
| 15 | 15.6 | 7.2 |
| 27 | 3.4 | 1.6 |
| 35 | 14.4 | 6.8 |
| 41 | 23.1 | 7.2 |
| 51 | <1 | 19.3 |
| 55 | 4.1 | <1.0 |
| 57 | 16.5 | 7.7 |
| 61 | 12.8 | 4.0 |
| 63 | 28.9 | 28.9 |
| 65 | 16.5 | 4.1 |
| 67 | 12.8 | 14.4 |
| 71 | 28.9 | 8.3 |
| 83 | 12.8 | 3.0 |
| 131 | 15.3 | 3.4 |
| 137 | 7.8 | 21.4 |
| 146 | 11.0 | 12.2 |

¹= comparison

ic) Solubility in Water at Different pH Values, Solubility [mg/mL]

For testing solubility, saturated solutions of selected compounds of formula I are prepared in four different systems and compared to the solubility of entacapone. The solubility is tested in 0.1 N HCl (pH 1), PBS buffer (pH 7.4) and in two simulated gastric fluids, FaSSIF at pH 6.5 (for fasting state) and FeSSIF at pH 5.0 (for fed state) at ambient temperature. The content of the FaSSIF simulated gastric fluid is as follows:

| | |
|---|---|
| NaH₂PO₄ | 3.9 g |
| NaCl | 6.2 g |
| sodium taurocholate | 3 mM |
| lecithin | 0.75 mM |
| distilled water | up to 1000 mL |
| NaOH | pH 6.5 |
| osmolarity | 270 ± 10 mOsm/L |

The content of the FeSSIF simulated gastric fluid is as follows:

| | |
|---|---|
| acetic acid | 8.65 g |
| NaCl | 1.19 g |
| sodium taurocholate | 15 mM |
| lecithin | 3.75 mM |
| distilled water | up to 1000 mL |
| NaOH | pH 5.0 |
| osmolarity | 635 ± 10 mOsm/L |

The solubility testing is carried out by the following method: An excess amount of each compound is added to a 0.1 N HCl, PBS buffer, FeSSIF and FaSSIF. The suspensions are shaken for 10 min. After centrifugation the concentration is determined by HPLC. Reversed phase gradient HPLC method, column YMC Pro C8, 50 mm×4.0 mm, 3 μm, Eluent A: Water:trifluoroacetic acid (100:0.5 (v/v)), Eluent B: Acetonitrile:trifluoroacetic:acid (100:0.5 (v/v)). Column temperature 30° C., Flow rate 3.0 mL/min, Detection wavelength: 220 nm

The results of the study are summarized in Table 3.

TABLE 3

| | Solubility | Solubility | Solubility | Solubility |
|---|---|---|---|---|
| | 0.1N HCl | FeSSIF | FaSSIF | PBS |
| | Ph 1.0 | Ph 5.0 | Ph 6.5 | Ph 7.4 |
| Compound | (mg/mL) | (mg/mL) | (mg/mL) | (mg/mL) |
| entacapone¹ | 0.05 | 0.99 | 0.15 | 1.35 |
| 1 | 0.02 | 0.20 | 0.53 | 0.53 |
| 7 | 0.003 | 0.09 | 0.75 | 0.95 |
| 10 | 0.03 | 0.19 | 0.84 | 1.33 |
| 11 | 0.15 | 0.02 | 0.08 | 0.28 |
| 15 | N/D | 0.19 | 0.18 | 0.15 |
| 35 | N/D | 0.15 | 0.15 | 0.13 |
| 41 | 0.01 | 0.20 | 0.29 | 0.47 |
| 51 | <0.01 | 0.14 | 1.2 | 0.81 |
| 55 | >1.0 | >1.0 | >1.0 | >1.0 |
| 57 | N/D | 0.03 | 0.14 | 0.26 |
| 61 | N/D | 0.04 | 0.24 | 0.49 |
| 63 | 0.02 | 0.17 | 0.75 | 1.23 |
| 71 | 0.07 | 1.1 | 0.57 | 0.8 |
| 83 | 0.01 | 0.43 | 0.91 | 0.85 |
| 131 | 0.02 | 0.71 | 0.96 | 0.97 |
| 137 | N/D | 0.02 | 0.15 | 0.34 |
| 146 | 0.03 | 0.25 | 0.63 | 0.83 |

¹= comparison
N/D = no compound detected

id) Log D (pH 7.4)

LogD is an indicator for absorption and lipophilicity for ionized compounds under defined pH conditions and is measured by applying an isocratic HPLC Method at pH 7.4.

The log D has been tested at pH 7.4. The testing has been carried out by the following method: Isocratic HPLC method: Column YMC Pro C18, 150 mm×4.0 mm, 3 μm, Eluent Phosphate buffer pH 7.4/acetonitrile (45/55, (v/v)), Runtime 15-60 min, Flow rate 1.2 ml/min, Detection wavelength: 220 nm

The results are summarized in Table 4.

TABLE 4

| Compound | LogD at pH 7.4 |
|---|---|
| entacapone¹ | 0.5 |
| 1 | 1.4 |
| 7 | 1.0 |
| 10 | 1.8 |
| 11 | 1.6 |
| 15 | 2.9 |
| 27 | >8 |
| 35 | 3.1 |
| 41 | 1.2 |
| 51 | 1.8 |
| 55 | 1.0 |
| 57 | 2.3 |
| 61 | 1.7 |
| 63 | 1.4 |
| 65 | 1.0 |
| 67 | 2.8 |
| 71 | 0.8 |
| 83 | 1.1 |
| 131 | 0.9 |
| 137 | 2.6 |
| 146 | 1.7 |

¹= comparison

ii) Permeability in Caco-2-Cells, Pₐₚₚ

The human adenocarcinoma cell line Caco-2 is used as an in vitro system to predict oral absorption. These cells serve as a model of small bowel tissue and permeation through these cells is a predictor for gastro-intestinal absorption. Further to being a model for oral absorption, permeability in Caco-2 cells may be predictive for the blood brain barrier permeability (P. Garberg et al.; Toxicology in vitro 19 (2005); 299-334). Permeability data in Caco-2 cells show that an increased oral bioavailability and brain permeability may be expected for the selected compounds of formula I (see Table 5). The permeability in Caco-2 cells has been tested by the following method:

The permeability of selected compounds of formula I across Caco-2 (21d Caco-2-culture) monolayer in the apical to basolateral direction is investigated. Control substances were ³H-propranolol and ³H-mannitol.

The nominal concentration of the test items is 20 mM. The pH value is set to 6.5 for the apical transport medium and to 7.4 for the basolateral medium. Samples are obtained following 60 minutes (120 minutes for control samples) incubation from the basolateral receiver chamber. At sampling time approximately 600 μL of each well are transferred into an eppendorf vial and stored between −12° and −30° C. until HPLC or LC-MS/MS analysis. The control substances are quantified by liquid scintillation. The Pₐₚₚ values are evaluated and summarized in Table 5. As shown in Table 5, the permeability data in Caco-2 cells demonstrate that some compounds of formula I may exhibit increased oral bioavailability and/or brain permeability.

TABLE 5

| Compound | Pₐₚₚ (10⁻⁶ cm/s) |
|---|---|
| entacapone¹ | 17 |
| 1 | 24 |
| 6 | <5.1 |
| 7 | 31 |
| 10 | 22.4 |
| 12 | 2.3 |
| 18 | <5.1 |
| 20 | 11.7 |
| 26 | <5.1 |
| 27 | 18.1 |
| 34 | 14.6 |
| 35 | >42 |
| 41 | 18 |
| 51 | 45 |
| 57 | 55 |
| 61 | 36 |
| 65 | 8.5 |
| 67 | 14.8 |
| 71 | 7.7 |
| 83 | 4.9 |

¹= comparison

Plasma stability is determined in human and rat plasma ex vivo. For the determination of the plasma stability of the compounds of formula I, the test compounds were incubated with human plasma (c=5 μg/mL) at 37° C. for certain time intervals up to 60, resp. 120 min (depending on the compound). Afterwards the incubations were stopped by equivalent addition of acetonitrile for protein precipitation, and the samples were mixed and centrifuged. The supernatants were adequately diluted and analyzed by HPLC with UV detection at 305 nm.

The degradation of the compounds of formula I as well as the formation of the active drug entacapone were monitored, and the half-life times of the compounds of formula I were calculated from the obtained peak areas. The results are presented in Table 6 for human plasma.

TABLE 6

| Compound | t_{1/2} plasma (min) |
|---|---|
| 1 | 7.7 |
| 7 | 13.4 |
| 15 | 88.8 |
| 27 | >8 |
| 35 | 119.8 |
| 41 | 6.7 |
| 51 | 38.4 |
| 57 | 18.1 |
| 61 | 28.2 |
| 65 | 1.0 |
| 67 | 2.8 |
| 71 | 0.8 |

iv) PK Profile (In Vivo)

The pharmacokinetics of entacapone and prodrug following the oral administration of selected compounds of formula I in rats and dogs are tested as described in Tables 7 (rats) and 8 (dogs) below.

TABLE 7

| | |
|---|---|
| Animals | Male Sprague-Dawley rats, n = 3 |
| Intravenous Dose | 2 mg/kg: entacapone and compounds 1, 7, 15, and 71 |
| (entacapone equivalents) | 3 mg/kg: entacapone and compound 7 |
| Oral Dose (entacapone | 10 mg/kg |
| equivalents) | |
| Formulation | 10% DMSO/90% phosphate buffer, Ph 7.4 |
| Blood Sampling (ca. | Serial blood samples are obtained via cannula inserted into |
| 0.3 ml) | lateral tail vein 2, 15, 30, 60, 120, 240, 360, and 480 min after |
| | intravenous dosing and 15, 30, 60, 90, 120, 240, 360, and |
| | 480 min after oral dosing. The samples are immediately |
| | centrifuged at 4° C. for plasma preparation, and are kept on ice |
| | until freezing for storage. |
| Sample Analysis | The chromatographic system used for LC-MS/MS analysis of |
| | the reference material, internal standard, and study samples |
| | consists of a Surveyor autosampler and HPLC pump linked |
| | to a Thermo TSQ Quantum triple quadrupole mass |
| | spectrometer operating in negative ion mode. Liquid |
| | chromatography is carried out with a Hypersil C18 BDS, |
| | 5 μm, 50 × 4.6 mm analytical column. The mobile phase is a |
| | 0.1% formic acid in water:0.1% formic acid in acetonitrile |
| | gradient. The flow rate is 1.0 ml/min and the injection |
| | volume is 10 μL. |
| Sample Preparation | Protein precipitation |
| PK Analysis | Cₘₐₓ, tₘₐₓ, AUC, t_{1/2}, CL (CL/F), Vd (Vd/F), and relative and |
| | absolute bioavailability |

TABLE 8

| | |
|---|---|
| Animals | Male Beagle dogs, n = 4 |
| Oral Dose (entacapone | 2.5 mg/kg, 5.0 mL/kg |
| equivalents) | |
| Formulation | phosphate buffer (pH 7.4), 0.5 mg/mL |
| Blood Sampling (ca. 2 ml) | Serial blood samples are obtained by direct venepuncture of a |
| | jugular vein at 5, 15, 30, and 60 min, and 2, 4, 6, and 8 h. The |
| | samples are handled at 4° C. |
| Sample Analysis: | The chromatographic system used for LC-MS/MS analysis of |
| | the reference material, internal standard, and study samples |
| | consists of a Surveyor autosampler and HPLC pump linked |
| | to a Thermo TSQ Quantum triple quadrupole mass |
| | spectrometer operating in negative ion mode. Liquid |
| | chromatography is carried out with a Hypersil C18 BDS, |
| | 5 μm, 50 × 4.6 mm analytical column. The mobile phase is a |
| | 0.1% formic acid in water:0.1% formic acid in acetonitrile |
| | gradient. The flow rate is 1.0 ml/min, and the injection |
| | volume is 10 μL. |
| Sample Preparation | Protein precipitation |
| PK Analysis: | Cₘₐₓ, tₘₐₓ, AUC, t_{1/2}, CL (CL/F), Vd (Vd/F) and relative |
| | bioavailability |

### Data Analysis:

The plasma concentration versus time curves obtained following intravenous and oral administration are analysed using WinNonLin (Version 4.1, Scientific Consulting Inc. USA). The kinetic data is characterised by non-compartmental analysis.

The following pharmacokinetic parameters are derived from the profiles: maximum peak plasma concentration (Cmax); the time of maximum observed concentration (Tmax); the terminal half life (t_{1/2}), area under the curve (AUC), total body clearance (CL or CL/F), volume of distribution (Vd or Vd/F), and bioavailability.

### Area Under the Curve (AUC)

The AUC is determined using the linear/log trapezoidal method. A value of zero is used for any plasma concentrations recorded as below the limits of quantification. The AUCinf (observed) is calculated as the area under the curve from the time of dosing extrapolated to time infinity based on the observed concentrations. The AUClast parameter is defined as the area under the curve from the point of dosing to the last measurable concentration.

AUCinf=AUClast+(Clast/λz)

### Where λz is the elimination constant and Clast is the last measurable concentration

### Clearance

The total body clearance (CL) is determined for both intravenous and oral administration (reported as CL/F). Clearance is calculated as:

$$Clearance ( CL or CL \text{/} F ) = ( \frac{Dose}{AUC inf} )$$

### Volume of Distribution

The volume of distribution (Vd) based on the terminal elimination phase is determined as:

$$Volume of distribution ( Vd \text{/} F ) = ( \frac{{Dose}_{po}}{λ z * AUCinf} )$$

### Where λz is the first order rate constant associated with the terminal (log-linear) portion of the plasma concentration time profile.

### Relative Bioavailability

The relative bioavailability [%] of entacapone following oral dosing of selected compounds of formula I is determined by the formula given below:

$$\% relative bioavailability = 100 \% * \frac{{[ AUC ]}_{A} * {dose}_{B}}{{[ AUC ]}_{B} * {dose}_{A}}$$

### In this formula, A is entacapone formed from a prodrug compound of formula I, and B is entacapone

### Absolute Bioavailability

The absolute bioavailability measures the availability of the active drug in systemic circulation after oral administration. In order to determine absolute bioavailability the pharmacokinetic study must be conducted following both intravenous (IV) and oral (po) administration. The absolute bioavailability is determined by the formula given below.

$$\% F = 100 \% * \frac{{[ AUC ]}_{po} * {dose}_{IV}}{{[ AUC ]}_{IV} * {dose}_{po}}$$

### The results of the studies concerning the plasma stability (in vivo) are summarized in Tables 9 (rats) and 10 (dogs).

TABLE 9

| | | | | | | Relative |
|---|---|---|---|---|---|---|
| | | | | | | Bio- |
| | | | | | AUC_{0→∞} | avail- |
| | Cₘₐₓ | tₘₐₓ | t_{1/2} | AUC_{0→8 h} | (ng/ | ability |
| Compound | (ng/mL) | (h) | (h) | (ng/mL * h) | mL * h) | (%) |
| entacapone¹ | 1836 | 0.25 | 1.84 | 1772 | 1800 | 100 |
| 1 | 1145 | 0.25 | 2.18 | 2115 | 2207 | 123 |
| 7 | 552 | 0.25 | 1.88 | 1040 | 1099 | 61 |
| 71 | 1635 | 0.25 | 2.15 | 2252 | 2354 | 131 |

¹= comparison

### As shown in Table 9, in rats the bioavailability of some compounds of formula I is up to about 1.3 times better than the bioavailability of entacapone.

TABLE 10

| | | | | | | Relative |
|---|---|---|---|---|---|---|
| | | | | | AUC_{0→∞} | Bioavail- |
| | Cₘₐₓ | tₘₐₓ | t_{1/2} | AUC_{0→8 h} | (ng/ | ability |
| Compound | (ng/mL) | (h) | (h) | (ng/mL * h) | mL * h) | (%) |
| entacapone¹ | 1031 | 0.25 | 2.0 | 757 | 772 | 100 |
| 1 | 768 | 0.25 | 0.9 | 631 | 650 | 83 |
| 7 | 944 | 0.50 | 2.3 | 1044 | 866² | 138 |
| 71 | 1589 | 0.25 | 1.1 | 1753 | 1818 | 232 |

¹comparison
²n = 2

As shown in Table 10, in dogs the bioavailability of some compounds of formula I is up to about 2.3 times the bioavailability of entacapone.

### FORMULATION EXPERIMENTS

Selected lipophilic carbonate compounds of formula I with promising Caco-2 permeabilities are formulated with different lipids/lipophilic excipients and/or micelles forming compounds in order to increase their solubilities. The following formulations are prepared by mixing the ingredients mentioned below:
i) Formulations comprising solubilizers in the form of lipides/lipophilic excipients:
ia) Formulation comprising Labrasol® and the selected compounds of Formula I
   Labrasol®:
   Caprylocaproyl macrogol-8-glyceride (CAS No. 85536-07-8 and 84963-88-2). This is a mixture of mono-, di- and triesters of glycerol and of PEG 400 with medium-chain fatty acids (C₈-C₁₀) which is marketed, for example, by Gattefossé under the mark Labrasol®; Labrasol® has an HLB value (hydrophil-lipophil-balance, Griffin, W. C.: Classification of surface active agents by HLB, J. Soc. Cosmet. 1, 1949) of 14 and has the following composition by weight:

| | |
|---|---|
| C₈-C₁₀ monoglycerides | approximately 4%; |
| C₈-C₁₀ diglycerides | approximately 17%; |
| C₈-C₁₀ triglycerides | approximately 6%; |
| C₈-C₁₀ monoesters of PEG 400 | approximately 14%; |
| C₈-C₁₀ diesters of PEG 400 | approximately 36%; |
| free PEG 400 | approximately 20%; |
| free glycerol | approximately 3%. |

ib) Formulation comprising NanoSolve® 5401 (Lipoid GmbH, Germany) and the selected compounds of formula I
ic) Formulation comprising TPGS/PG in a weight ratio of 25% by weight of TPGS to 75% by weight of PG and the selected compounds of formula I.
   PG: propylene glycol
   TPGS: Vitamin E TPGS NF (d-α-tocopheryl polyethylene glycol 1000 succinate) which is marketed, for example, by Eastman Chemical Co
ii) Formulation comprising a micelles forming agent:
iia) Aqueous formulation comprising 30% by weight of Cremophor® RH 40 (relating to the weight of the aqueous phase) in water and the selected compounds of formula I
   Cremophor® RH 40:
   Generic names:
   Polyoxyl 40 Hydrogenated Castor Oil (USP/NF).
   Macrogol-Glycerolhydroxystearat (DAB).
   Polyoxyethylenglyceroltrihydroxystearat (DAC).
   Cremophor RH 40 is a non-ionic solubilizer and emulsifying agent obtained by reacting 45 moles of ethylene oxide with 1 mole of hydrogenated castor oil. The main constituent of Cremophor RH 40 is glycerol polyethylene glycol oxystearate, which, together with fatty acid glycerol polyglycol esters, forms the hydrophobic part of the product. The hydrophilic part consists of polyethylene glycols and glycerol ethoxylate. Cremophor RH 40 is a white to yellowish thin paste at 20° C. The HLB value lies between 14 and 16. Cremophor RH 40 is marketed by BASF AG.

The solubility of selected compounds of formula I in these formulations is measured and compared to the solubility of the compounds in 0.1N HCl (pH 1.0), PBS buffer (pH 7.4), and two simulated gastric fluids (i.e., FaSSIF (pH 6.5) simulating fasting sate and FeSSIF (pH 5.0) simulating fed state) (see above for details). The results are presented in Table 11.

TABLE 11

| | Solubility² | Solubility | Solubility | Solubility | | |
|---|---|---|---|---|---|---|
| | 0.1N HCl | PBS | FaSSIF | FeSSIF | Solubility | Solubility |
| | pH 1.0 | pH 7.4 | pH 6.5 | pH 5.0 | Labrasol ® | TPGS/PG |
| Compound | (mg/mL) | (mg/mL) | (mg/mL) | (mg/mL) | (mg/mL) | (mg/mL) |
| entacapone¹ | 0.05 | 1.35 | 0.15 | 0.99 | 23 | N/A |
| 10 | 0.03 | 0.19 | 0.84 | 1.33 | 22 | 3.9 |
| 15 | N/D | 0.15 | 0.18 | 0.19 | 50 | 2.4 |
| 35 | N/D | 0.13 | 0.15 | 0.15 | >36 | 2.3 |
| 57 | N/D | 0.26 | 0.14 | 0.03 | 22 | 2.5 |
| 63 | 0.02 | 1.23 | 0.75 | 0.17 | 10 | 1.8 |

¹= comparison
²= maximum solubility
N/D = no compound detected
N/A = not analyzed

### Stability in TPGS/PG Over 22 h

The stability of selected compounds of formula I in TPGS/PG over 22 h is tested by the following procedure:

37.5 mg of compounds 15, 35 and 57, respectively 32 mg of compound 63, are in each case added to 25 mL of TPGS/PG (25% by weight of TPGS, 75% by weight of PG) (concentration of compounds 15, 35, 57:1.5 mg/mL; concentration of compound 63:1.4 mg/mL). Each formulation obtained is stirred for 22 h at ambient temperature. The amount of the respective compound of formula I and the amount of entacapone are determined after 5 h, 8 h, 11 h, and 22 h by HPLC assay. Reversed phase gradient HPLC method, column YMC Pro C8, 50 mm×4.0 mm, 3 μm, Eluent A: Water:trifluoroacetic acid (100:0.5 (v/v)), Eluent B: Acetonitrile:trifluoroacetic acid (100:0.5 (v/v)). Column temperature 30° C., Flow rate 3.0 mL/min, Detection wavelength: 220 nm

As shown in Table 12, the compounds are stable in the assay performed.

TABLE 12

| | | Amount of compound | Amount of entacapone |
|---|---|---|---|
| Compound | Time (h) | (mg/mL) | (mg/mL) |
| 15 | 5 | 1.23 | 0.05 |
| | 8 | 1.23 | 0.06 |
| | 11 | 1.16 | 0.10 |
| | 22 | 1.05 | 0.17 |
| 35 | 5 | 1.21 | 0.03 |
| | 8 | 1.36 | 0.05 |
| | 11 | 1.40 | 0.07 |
| | 22 | 1.23 | 0.08 |
| 57 | 5 | 1.37 | 0.03 |
| | 8 | 1.39 | 0.04 |
| | 11 | 1.35 | 0.05 |
| | 22 | 1.31 | 0.08 |
| 63 | 5 | 1.25 | 0.02 |
| | 8 | 1.12 | 0.02 |
| | 11 | 1.16 | 0.02 |
| | 22 | 1.08 | 0.03 |

### Stability of Formulations Comprising Compounds and Salts of the Invention in Physiological Fluids.

Simulated gastric fluid (SGF) is prepared as follows. 200 mg NaCl are dissolved in 70 mL H2O. 0.7 mL concentrated HCl is added. Subsequently, 320 mg of pepsin powder is added, and the mixture is filled up to 100 mL with H2O. A clear solution is obtained (pH 1-2).

### Stability of Labrasol® Formulations.

1.5 mL of Labrasol® is added to 3 mg of the respective compound of formula I, and the obtained solutions are shaken. Subsequently, each of the solutions is added to 10 mL SGF by drop-wise addition. Precipitate formation is determined by visual inspection of each solution immediately after the addition of the solution comprising the respective compound of formula I in Labrasol® to SGF and after 5, 15, 30, and 60 min, and 24 h.

The results are presented in table 13. As shown in Table 13, the formulations of the compounds of formula I in Labrasol® are stable in SGF.

TABLE 13

| Com- | | | 15 | 30 | | |
|---|---|---|---|---|---|---|
| pound | Directly after addition | 5 min | min | min | 60 min | 24 h |
| 15 | First: white precipitate; | clear | clear | clear | clear | clear |
| | clear after shaking | | | | | |
| 35 | First: white precipitate; | clear | clear | clear | clear | clear |
| | clear after shaking | | | | | |
| 57 | First: white precipitate; | clear | clear | clear | clear | clear |
| | clear after shaking | | | | | |
| 63 | First: white precipitate; | clear | clear | clear | clear | clear |
| | clear after shaking | | | | | |

### Stability of TPGS/PG Formulations.

2 mL TPGS/PG is added to 5-6 mg of the respective compound of formula I, and the obtained solutions are shaken for 5 h. Since the selected compounds of formula I do not completely dissolve in the solvent, the excess fluid is decanted, and then centrifuged. 1.0 mL of the centrifuged solution is added drop-wise to 10 mL SGF. Precipitate formation is determined by visual inspection of each solution immediately after addition of the solution comprising the respective compound of formula I in TPGS/PG to SGF and after 24 hours. The results are presented in Table 14. As shown in Table 14, the formulations of the compounds of formula I in TPGS/PG are stable in SGF.

TABLE 14

| Compound | Directly after addition | 24 h |
|---|---|---|
| 15 | clear | clear |
| 35 | clear | clear |
| 57 | clear | clear |
| 63 | clear | clear |

### Comparison of the Pharmacokinetic Profile of Compound 15 in Labrasol® or TPSG/PG to Its Pharmacokinetic Profile in Phosphate Buffer (pH 7.4).

The pharmacokinetics of compound 15 in rats following oral administration is tested as described in Table 15 below.

TABLE 15

| | |
|---|---|
| Animals | Male Sprague-Dawley rats, n = 6 |
| Oral Dose (entacapone | 10 mg/kg |
| equivalents) | |
| Formulation | i) Labrasol ®, 5 mL/kg |
| | ii) TPSG/PG (25% by weight TPGS, 75% by weight of PG) |
| | iii) Phosphate buffer, pH 7.4 |
| Blood Sampling (ca. | Blood samples are obtained 15, 30, 60, 120, 240, 360, and |
| 0.3 ml) | 480 minutes after oral dosing. The samples are immediately |
| | centrifuged at 4° C. for plasma preparation, and are kept on ice |
| | until freezing for storage. |
| Sample Analysis | The chromatographic system used for LC-MS/MS analysis of |
| | the reference material, internal standard, and study samples |
| | consists of a Surveyor autosampler and HPLC pump linked |
| | to a Thermo TSQ Quantum triple quadrupole mass |
| | spectrometer operating in negative ion mode. Liquid |
| | chromatography is carried out with a Hypersil C18 BDS, |
| | 5 μm, 50 × 4.6 mm analytical column. The mobile phase is a |
| | 0.1% formic acid in water:0.1% formic acid in acetonitrile |
| | gradient. The flow rate is 1.0 ml/min and the injection |
| | volume is 10 μL. |
| Sample Preparation | Protein precipitation |
| PK Analysis | Cₘₐₓ, tₘₐₓ, AUC, t_{1/2}, and relative bioavailability |

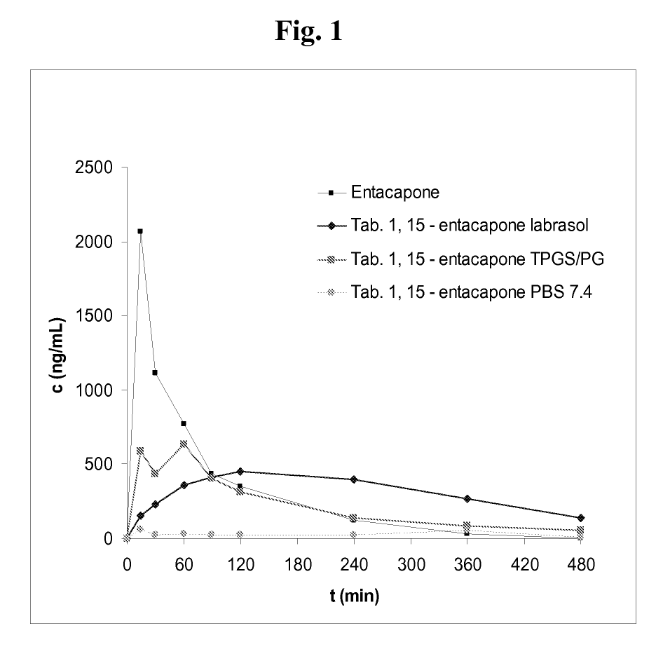

The results are summarized in FIG. 1 (The symbols in FIG. 1 have the following meanings: c (ng/mL) concentration in ng/mL, t (min) time in minutes). FIG. 1 shows how much entacapone is present in rat plasma after oral administration of compound 15 formulated in (i) Labrasol®; (ii) TPGS/PG; or (iii) phosphate buffer (pH 7.4). Entacapone concentration is shown in ng/mL, and time is shown in minutes. As can be seen from FIG. 1, the plasma level of entacapone formed after administration of compound 15 in either a Labrasol® or TPGS/PG formulation is more constant than the plasma level of entacapone formed after administration of compound 15 in phosphate buffer.

We claim:

1. A pharmaceutical composition comprising a compound and one or more pharmaceutically acceptable carriers, wherein the compound is selected from those of formula I, salts thereof, and double bond configurational isomers thereof, 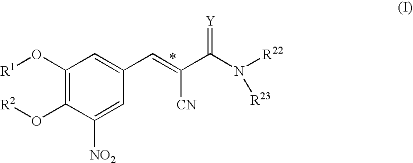
wherein
the double bond marked with an asterisk is in the E- or Z-configuration;
Y is sulfur or oxygen;
R¹ is a group of the following formula II 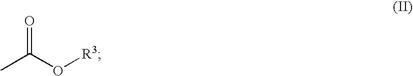
R² is H or a group of formula II which may be the same as or different from R¹;
each R³ is an organic group with 1 to 30 non-hydrogen atoms; and
R²² and R²³ are independently selected from the group consisting of H and (C₁-C₁₅)-alkyl;
with the proviso that, when R² is H, R²² and R²³ are ethyl, R³ is t-butyl, and the asterisked double bond is in the E-configuration, Y is not O.

2. A composition according to claim 1, wherein
each R³ is independently (C₁-C₂₀)-alkyl, (CR⁴R⁵)ₓ—R⁶, (C₁-C₂₀)-alkylene-(C₁-C₂₀)-alkoxy, (C₂-C₂₀)-alkenyl, (C₂-C₂₀)-alkynyl, (C₀-C₂₀)-alkylene-(C₃-C₁₈)-cycloalkyl, (C₀-C₂₀)-alkylene-(3-18-membered)-heterocycloalkyl, (C₁-C₂₀)-alkylene-(C₃-C₁₈)-cycloalkenyl, (C₀-C₂₀)-alkylene-(3-18-membered)-heterocycloalkenyl, (C₀-C₂₀)-alkylene-(C₆-C₁₈)-aryl, (C₀-C₂₀)-alkylene-(5-18-membered)-heteroaryl, (C₂-C₂₀)-alkenylene-(C₃-C₁₋₈)-cycloalkyl, (C₂-C₂₀)-alkenylene-(3-18-membered)-heterocycloalkyl, (C₂-C₂₀)-alkenylene-(C₃-C₁₈)-cycloalkenyl, (C₂-C₂₀)-alkenylene-(3-18-membered)-heterocycloalkenyl, (C₂-C₂₀)-alkenylene-(C₆-C₁₈)-aryl, or (C₂-C₂₀)-alkenylene-(5-18-membered)-heteroaryl;
each R⁴ and R⁵ are independently of one another selected from the group consisting of H, (C₁-C₂₀)-alkyl, (C₁-C₂₀)-alkylene-hydroxy, (C₀-C₂₀)-alkylene-(C₁-C₂₀)-alkoxy, OH, (C₀-C₂₀)-alkylene-N(R⁷)CO—(C₁-C₂₀)-alkyl, (C₀-C₂₀)-alkylene-CON(R⁸)(R⁹), (C₀-C₂₀)-alkylene-COO—(C₁-C₂₀)-alkyl, (C₀-C₂₀)-alkylene-N(R¹⁰)(R¹¹), SO₃R¹⁷, (C₀-C₂₀)-alkylene-(C₆-C₁₈)-aryl, and (C₀-C₂₀)-alkylene-(5-18-membered)-heteroaryl, or
R⁴ and R⁵ of the same group (CR⁴R⁵) or R⁴ and R⁵ of different groups (CR⁴R⁵) form together a carbocyclic or heterocyclic ring having from 3 to 6 atoms, additionally, one or more non adjacent groups (CR⁴R⁵) may be replaced by O, CO, OCO, COO, CON(R¹⁹), N(R²⁰)CO, or NR²¹;
R⁶ is independently H, (C₁-C₂₀)-alkyl, (C₂-C₂₀)-alkenyl, (C₂-C₂₀)-alkynyl, OH, O—(C₁-C₈)-alkyl, O—(C₀-C₈)-alkylene-(C₆-C₁₄)-aryl, CO—O—(C₁-C₈)-alkyl, CO—N(R¹²)(R¹³), N(R¹⁴)CO—(C₁-C₈)-alkyl, N(R¹⁵)(R¹⁶), SO₃R¹⁸, (C₀-C₂₀)-alkylene-(5-18-membered)-heteroaryl, or (C₀-C₂₀)-alkylene-(C₆-C₁₈)-aryl;
R⁷, R¹⁴, R¹⁷, R¹⁸, R¹⁹, R²⁰, R²¹ are independently of one another H, or (C₁-C₂₀)-alkyl;
R⁸, R⁹, R¹⁰, R¹¹, R¹², R¹³, R¹⁵, R¹⁶ are independently of one another H, or (C₁-C₂₀)-alkyl;
R²² and R²³ are independently selected from the group consisting of H and (C₁-C₁₅)-alkyl; and
x is 1 to 14;
wherein the alkyl, alkenyl, alkynyl, cycloalkyl, heterocycloalkyl, cycloalkenyl, heterocycloalkenyl, alkoxy, aryl, heteroaryl, alkenylene and alkylene groups are unsubstituted or further substituted.

3. A pharmaceutical composition according to claim 1, wherein Y is oxygen, and R²² and R²³ are ethyl.

4. A pharmaceutical composition according to claim 2, wherein the total number of carbon atoms of R³ is at most 25,
R⁴ and R⁵ are independently of one another selected from the group consisting of H, (C₁-C₁₅)-alkyl, (C₁-C₁₅)-alkylene-hydroxy, (C₀-C₁₅)-alkylene-(C₁-C₁₅)-alkoxy, OH, (C₀-C₁₅)-alkylene-N(R⁷)CO—(C₁-C₁₅)-alkyl, (C₀-C₁₅)-alkylene-CON(R⁸)(R⁹), (C₀-C₁₅)-alkylene-COO—(C₁-C₁₅)-alkyl, (C₀-C₁₅)-alkylene-N(R¹⁰)(R¹¹), SO₃R¹⁷, (C₀-C₁₅)-alkylene-(C₆-C₁₈)-aryl, and (C₀-C₁₅)-alkylene-(5-18-membered)-heteroaryl, or
R⁴ and R⁵ of the same group (CR⁴R⁵) or R⁴ and R⁵ of different groups (CR⁴R⁵) form together a carbocyclic or heterocyclic ring having from 3 to 6 atoms, wherein one or more non-adjacent groups (CR⁴R⁵) are optionally replaced by O, CO, OCO, COO, CON(R¹⁹), N(R²⁰)CO, or NR²¹;
R⁶ is independently H, (C₁-C₁₅)-alkyl, (C₂-C₂₀)-alkenyl, (C₂-C₁₅)-alkynyl, OH, O—(C₁-C₈)-alkyl, O—(C₀-C₈)-alkylene-(C₆-C₁₄)-aryl, CO—O—(C₁-C₈)-alkyl, CO—N(R¹²)(R¹³), N(R¹⁴)CO—(C₁-C₈)-alkyl, N(R¹⁵)(R⁶), SO₃R¹⁸, (C₀-C₁₅)-alkylene-(5-18-membered)-heteroaryl, or (C₀-C₁₅)-alkylene-(C₆-C₁₈)-aryl;
R⁷, R¹⁴, R¹⁷, R¹⁸, R¹⁹, R²⁰, R²¹ are independently of one another H, or (C₁-C₁₅)-alkyl; and
R⁸, R⁹, R¹⁰, R¹¹, R¹², R¹³, R¹⁵, R¹⁶ are independently of one another H, or (C₁-C₁₅)-alkyl.

5. A pharmaceutical composition according to claim 1, wherein in the compound of formula I
the total number of carbon atoms of R³ is at most 15,
each R⁴ and R⁵ are independently of one another selected from the group consisting of H, (C₁-C₈)-alkyl, (C₁-C₈)-alkylene-hydroxy, (C₀-C₈)-alkylene-(C₁-C₄)-alkoxy, OH, (C₀-C₈)-alkylene-N(R⁷)CO—(C₁-C₈)-alkyl, (C₀-C₈)-alkylene-CO—N(R⁸)(R⁹), (C₀-C₈)-alkylene-COO—(C₁-C₈)-alkyl, (C₀-C₈)-alkylene-N(R¹⁰)(R¹¹), and (C₀-C₈)-alkylene-(C₆-C₁₄)-aryl, or
R⁴ and R⁵ of the same group (CR⁴R⁵) or R⁴ and R⁵ of different groups (CR⁴R⁵) form together a carbocyclic or heterocyclic ring having from 3 to 6 atoms, wherein one or more non adjacent groups (CR⁴R⁵) are optionally replaced by CO, O, OCO, COO, CON(R¹⁹), or N(R²⁰)CO;
R⁶ is independently H, OH, O—(C₁-C₈)-alkyl, O—(C₀-C₈)-alkylene-(C₆-C₁₄)-aryl, CO—O—(C₁-C₈)-alkyl, CO—N(R¹²)(R¹³), N(R¹⁴)CO—(C₀-C₈)-alkyl, N(R¹⁵)(R¹⁶), or SO₃R¹⁸;
R⁷, R¹⁴, R¹⁷, R¹⁹ and R²⁰ are independently of one another H, or (C₁-C₈)-alkyl;
R⁸, R⁹, R¹⁰, R¹¹, R¹², R¹³, R¹⁵, and R¹⁶ are independently of one another H, or (C₁-C₈)-alkyl; and
x is 1 to 8.

6. A pharmaceutical composition according to claim 1, wherein in the compound of formula I R² is H.

7. A pharmaceutical composition according to claim 1, wherein in the case that R² is H, R³ in the residue R¹ is not tert-butyl.

8. A pharmaceutical composition according to claim 1, wherein in the compound of formula I each R³ is independently (C₁-C₁₀)-alkyl, (CR⁴R⁵)ₓ—R⁶, (C₃-C₂₀)-alkenyl, (C₃-C₈)-alkynyl, (C₀-C₈)-alkylene-(C₃-C₁₄)-cycloalkyl, (C₀-C₈)-alkylene-(3-14-membered)-heterocycloalkyl, (C₀-C₈)-alkylene-(C₆-C₁₄)-aryl, or (C₀-C₈)-alkylene-(5-14-membered)-heteroaryl, wherein the total carbon number of R³ is at most 15, (CR⁴R⁵)ₓ is (C₁-C₄)-alkylene, and R⁶ is independently CO—O—(C₁-C₄)-alkyl or CO—N(R¹²)(R¹³).

9. A pharmaceutical composition according to claim 1, wherein each R³ is independently selected from the group consisting of (C₁-C₄)-alkyl, (C₅-C₇)-alkyl, and (C₈-C₂₀)-alkyl.

10. A pharmaceutical composition according to claim 9, wherein R³ is selected from the group consisting of ethyl, isopropyl, isobutyl, 1-ethyl-propyl and 2-ethylhexyl.

11. A pharmaceutical composition according to claim 1, further comprising L-dopa.

12. A pharmaceutical composition according to claim 11, further comprising a decarboxylase inhibitor.

13. A method for treating or preventing a disease state associated with a disordered dopamine metabolism or an altered COMT activity, the method comprising administering to a patient in need of such treatment a pharmaceutical composition according to claim 1.

14. A method according to claim 13, wherein the disease state is one of Parkinson's disease, psychosis, schizophrenia, mood disorders, depression, anxiety disorders, obsessional compulsive disorders, generalized anxiety, aggressive disorders, mixed aggressive-anxiety/depressive disorders, restless leg syndrome, dopa-sensitive dyskinesia, apraxia induced by dopa or neuroleptica, neurodegenerative disorders, cognitive disorders, attention deficit hyperactivity disorder (ADHD), heart failure, and hypertension.

15. A method for treating or preventing a disease state associated with a disordered dopamine metabolism or an altered COMT activity, the method comprising administering to a patient in need of such treatment a pharmaceutical composition according to claim 11.

16. A method according to claim 15, wherein the disease state is one of Parkinson's disease, psychosis, schizophrenia, mood disorders, depression, anxiety disorders, obsessional compulsive disorders, generalized anxiety, aggressive disorders, mixed aggressive-anxiety/depressive disorders, restless leg syndrome, dopa-sensitive dyskinesia, apraxia induced by dopa or neuroleptica, neurodegenerative disorders, cognitive disorders, attention deficit hyperactivity disorder (ADHD), heart failure, and hypertension.

17. A compound of formula I, a salt thereof, an enantiomer thereof, a positional isomer thereof, or a configurational isomer thereof, 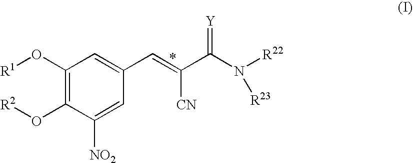
wherein
the double bond marked with an asterisk is in the E- or Z-configuration;
Y is sulfur or oxygen;
R¹ is a group of the following formula II 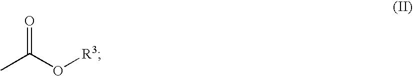
R² is H or a group of formula II which may be the same as or different from R¹;
each R³ is an organic group with 1 to 30 non-hydrogen atoms; and
R²² and R²³ are independently selected from the group consisting of H and (C₁-C₁₅)-alkyl;
with the proviso that, when R² is H, R²² and R²³ are ethyl, R³ is t-butyl, and the asterisked double bond is in the E-configuration, Y is not O.

18. A compound according to claim 17, wherein R² is H.

19. A compound according to claim 17, wherein the double bond is in the E configuration.

20. A compound according to claim 17, wherein Y is O.

21. A compound according to claim 17, wherein R² is a group of Formula II.

22. The compound according to claim 17, selected from the group consisting of:
Carbonic acid 5-((E)-2-cyano-2-diethylcarbamoyl-vinyl)-2-hydroxy-3-nitro-phenyl ester isobutyl ester,
Carbonic acid 5-((E)-2-cyano-2-diethylcarbamoyl-vinyl)-2-hydroxy-3-nitro-phenyl ester phenethyl ester,
Carbonic acid 5-((E)-2-cyano-2-diethylcarbamoyl-vinyl)-2-hydroxy-3-nitro-phenyl ester pentyl ester,
Carbonic acid 5-((E)-2-cyano-2-diethylcarbamoyl-vinyl)-2-hydroxy-3-nitro-phenyl ester 1-methyl-pentyl ester,
Carbonic acid 5-((E)-2-cyano-2-diethylcarbamoyl-vinyl)-2-hydroxy-3-nitro-phenyl ester 1-ethyl-propyl ester,
Carbonic acid 5-((E)-2-cyano-2-diethylcarbamoyl-vinyl)-2-hydroxy-3-nitro-phenyl ester 2-ethyl-hexyl ester,
Carbonic acid butyl ester 5-((E)-2-cyano-2-diethylcarbamoyl-vinyl)-2-hydroxy-3-nitro-phenyl ester,
2-[5-((E)-2-Cyano-2-diethylcarbamoyl-vinyl)-2-hydroxy-3-nitro-phenoxycarbonyloxy]-2-methyl-propionic acid methyl ester,
Carbonic acid 5-((E)-2-cyano-2-diethylcarbamoyl-vinyl)-2-hydroxy-3-nitro-phenyl ester 8-diethylamino-octyl ester,
Carbonic acid 4-((E)-2-cyano-2-diethylcarbamoyl-vinyl)-2-nitro-6-p-tolyloxycarbonyloxy-phenyl ester p-tolyl ester,
Carbonic acid 4-((E)-2-cyano-2-diethylcarbamoyl-vinyl)-2-nitro-6-phenoxycarbonyloxy-phenyl ester phenyl ester,
Carbonic acid 5-((E)-2-cyano-2-diethylcarbamoyl-vinyl)-2-hydroxy-3-nitro-phenyl ester octyl ester,
Carbonic acid 4-((E)-2-cyano-2-diethylcarbamoyl-vinyl)-2-nitro-6-octyloxycarbonyloxy-phenyl ester octyl ester,
Carbonic acid 5-((E)-2-cyano-2-diethylcarbamoyl-vinyl)-2-hydroxy-3-nitro-phenyl ester propyl ester,
Carbonic acid 4-((E)-2-cyano-2-diethylcarbamoyl-vinyl)-2-nitro-6-propoxycarbonyloxy-phenyl ester propyl ester,
Carbonic acid 4-((E)-2-cyano-2-diethylcarbamoyl-vinyl)-2-(2-methoxy-phenoxycarbonyloxy)-6-nitro-phenyl ester 2-methoxy-phenyl ester,
Carbonic acid 4-((E)-2-cyano-2-diethylcarbamoyl-vinyl)-2-(4-methoxy-phenoxycarbonyloxy)-6-nitro-phenyl ester 4-methoxy-phenyl ester,
3-[5-((E)-2-Cyano-2-diethylcarbamoyl-vinyl)-2-hydroxy-3-nitro-phenoxycarbonyloxy]-2-methylene-butyric acid methyl ester,
Carbonic acid sec-butyl ester 5-((E)-2-cyano-2-diethylcarbamoyl-vinyl)-2-hydroxy-3-nitro-phenyl ester,
3-[5-((E)-2-Cyano-2-diethylcarbamoyl-vinyl)-2-hydroxy-3-nitro-phenoxycarbonyloxy]-pentanedioic acid diethyl ester,
Carbonic acid 5-((E)-2-cyano-2-diethylcarbamoyl-vinyl)-2-hydroxy-3-nitro-phenyl ester cyclohexylmethyl ester,
Carbonic acid 5-((E)-2-cyano-2-diethylcarbamoyl-vinyl)-2-hydroxy-3-nitro-phenyl ester (E)-octadec-9-enyl ester,
Carbonic acid 5-((E)-2-cyano-2-diethylcarbamoyl-vinyl)-2-hydroxy-3-nitro-phenyl ester 2-thiophen-2-yl-ethyl ester,
Carbonic acid 5-((E)-2-cyano-2-diethylcarbamoyl-vinyl)-2-hydroxy-3-nitro-phenyl ester 1-methyl-butyl ester,
Carbonic acid allyl ester 5-((E)-2-cyano-2-diethylcarbamoyl-vinyl)-2-hydroxy-3-nitro-phenyl ester,
Carbonic acid 5-((E)-2-cyano-2-diethylcarbamoyl-vinyl)-2-hydroxy-3-nitro-phenyl ester 1,7,7-trimethyl-bicyclo[2.2.1]hept-2-yl ester,
Carbonic acid 5-((E)-2-cyano-2-diethylcarbamoyl-vinyl)-2-hydroxy-3-nitro-phenyl ester (S)-3,7-dimethyl-oct-6-enyl ester,
Carbonic acid 5-((E)-2-cyano-2-diethylcarbamoyl-vinyl)-2-hydroxy-3-nitro-phenyl ester ethyl ester,
2-[5-((E)-2-Cyano-2-diethylcarbamoyl-vinyl)-2-hydroxy-3-nitro-phenoxycarbonyloxy]-butyric acid methyl ester,
Carbonic acid 5-((E)-2-cyano-2-diethylcarbamoyl-vinyl)-2-hydroxy-3-nitro-phenyl ester 2-isopropoxy-ethyl ester,
Carbonic acid 2-tert-butoxy-ethyl ester 5-((E)-2-cyano-2-diethylcarbamoyl-vinyl)-2-hydroxy-3-nitro-phenyl ester,
Carbonic acid benzyl ester 2-benzyloxycarbonyloxy-4-((E)-2-cyano-2-diethylcarbamoyl-vinyl)-6-nitro-phenyl ester,
Carbonic acid 5-((Z)-2-cyano-2-diethylcarbamoyl-vinyl)-2-hydroxy-3-nitro-phenyl ester 2-ethyl-hexyl ester,
Carbonic acid 5-((E)-2-cyano-2-diethylcarbamoyl-vinyl)-2-hydroxy-3-nitro-phenyl ester tetrahydro-furan-2-ylmethyl ester,
Carbonic acid 5-((E)-2-cyano-2-diethylcarbamoyl-vinyl)-2-hydroxy-3-nitro-phenyl ester 4-methyl-cyclohexyl ester,
Carbonic acid 5-((E)-2-cyano-2-diethylcarbamoyl-vinyl)-2-hydroxy-3-nitro-phenyl ester 2-methyl-cyclohexyl ester,
Carbonic acid 5-((E)-2-cyano-2-diethylcarbamoyl-vinyl)-2-hydroxy-3-nitro-phenyl ester 2,5-dimethyl-cyclohexyl ester,
Carbonic acid bicyclo[2.2.1]hept-2-yl ester 5-((E)-2-cyano-2-diethylcarbamoyl-vinyl)-2-hydroxy-3-nitro-phenyl ester,
Carbonic acid 5-((E)-2-cyano-2-diethylcarbamoyl-vinyl)-2-hydroxy-3-nitro-phenyl ester 2-(2-methoxy-phenyl)-ethyl ester,
Carbonic acid 5-((E)-2-cyano-2-diethylcarbamoyl-vinyl)-2-hydroxy-3-nitro-phenyl ester 2-(3-methoxy-phenyl)-ethyl ester,
3-[5-((E)-2-Cyano-2-diethylcarbamoyl-vinyl)-2-hydroxy-3-nitro-phenoxycarbonyloxy]-butyric acid tert-butyl ester,
Carbonic acid 5-((E)-2-cyano-2-diethylcarbamoyl-vinyl)-2-hydroxy-3-nitro-phenyl ester 2-(4-methoxy-phenyl)-1-methyl-ethyl ester,
Carbonic acid 5-((E)-2-cyano-2-diethylcarbamoyl-vinyl)-2-hydroxy-3-nitro-phenyl ester 1-methyl-2-phenyl-ethyl ester,
Carbonic acid 5-((E)-2-cyano-2-diethylcarbamoyl-vinyl)-2-hydroxy-3-nitro-phenyl ester 3,5-dimethyl-cyclohexyl ester,
Carbonic acid 5-((E)-2-cyano-2-diethylcarbamoyl-vinyl)-2-hydroxy-3-nitro-phenyl ester 2-(2-propoxy-ethoxy)-ethyl ester,
3-[5-((E)-2-Cyano-2-diethylcarbamoyl-vinyl)-2-hydroxy-3-nitro-phenoxycarbonyloxy]-butyric acid ethyl ester, and
2-[5-((E)-2-Cyano-2-diethylcarbamoyl-vinyl)-2-hydroxy-3-nitro-phenoxycarbonyloxy]-cyclopentanecarboxylic acid methyl ester.

23. A process for the preparation of a compound of claim 17, comprising reacting a hydroxyl intermediate with phosgene to form a cyclic ester and selectively ring-opening the cyclic ester via alcoholysis.

24. A process for the preparation of a compound of claim 17, comprising reacting a hydroxyl intermediate with a chloroformic ester.

25. A process for the preparation of a compound of claim 17, comprising reacting a hydroxyl intermediate with pyrocarbonate.

26. A process for the preparation of a 2-cyanopropenamide compound in the form of its E-isomer comprising
reacting an aldehyde of formula (III) with N,N-dialkylcyanoacetamide (IV) in the presence of ammonium acetate, piperidine, or β-alanine, whereby the E-isomer formula (V) is obtained, 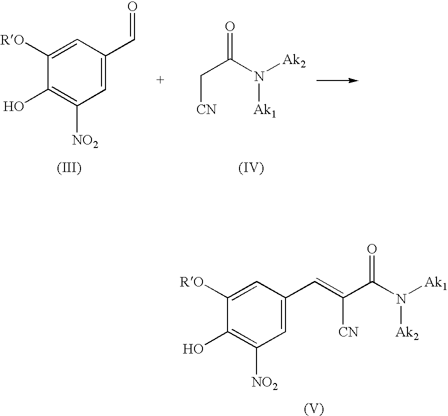
wherein R′ is methyl or ethyl and Ak₁ and Ak₂ are independently H or (C₁-C₁₅)-alkyl, and wherein the ammonium acetate, piperidine, or β-alanine is employed in a molar excess in relation to the aldehyde of formula III.

27. The process according to claim 26, further comprising
(i) preparing compound (IV) by deprotonating a dialkylamine with a lithium base followed by reacting the deprotonated base with ethylcyanoacetate;
(ii) reacting 5-nitrovanillin with compound (IV) in the presence of ammonium acetate, whereby an intermediate of formula (Va) is obtained, 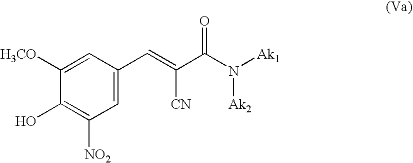
wherein ammonium acetate is employed in a molar excess in relation to the 5-nitrovanillin; and
(iii) reacting the intermediate of formula Va with AlCl₃ and pyridine in chloroform, whereby the E-isomer of formula (V) is obtained. [chemical structure]
